(19) United States
(12) Patent Application Publication
(10) Pub. No.: US 2025/0223651 A1
(43) Pub. Date: Jul. 10, 2025

(54) METHODS AND COMPOSITIONS FOR TREATMENT OF MEDULLOBLASTOMA

(71) Applicant: Children's Hospital Medical Center, Cincinnati, OH (US)

(72) Inventor: Qing Lu, Cincinnati, OH (US)

(21) Appl. No.: 18/960,079

(22) Filed: Nov. 26, 2024

Related U.S. Application Data
(60) Provisional application No. 63/603,244, filed on Nov. 28, 2023.

(51) Int. Cl. C12Q 1/6886

(52) U.S. Cl. CPC C12Q 1/6886; C12Q 2600/112; C12Q 2600/158

(57) ABSTRACT

Disclosed are methods of treating a medulloblastoma (MB) tumor in an individual in need thereof, comprising administering to the individual a composition comprising one or more of a nucleic acid having specific binding to a SOX11 mRNA, a nucleic acid having specific binding to an HNRNPH1 mRNA and/or a nucleic acid having specific binding to a MYC enhancer region. In aspects, the nucleic acid has specific binding to a MYC enhancer region and impedes binding of one or both of a SOX11 protein and a HNRNPH1 protein to the MYC enhancer region. CRISPRi and/or CRISPR methods may be used for delivery of the nucleic acids contemplated herein.

### CROSS-REFERENCE TO RELATED APPLICATIONS

This application claims priority to and benefit of U.S. Provisional Application Ser. No. 63/603,244, filed Nov. 28, 2023, the contents of which are incorporated in their entirety for all purposes.

### REFERENCE TO SEQUENCE LISTING

A Sequence Listing submitted as an ST.26 XML file via Patent Center is hereby incorporated by reference. The name of the XML file for the Sequence Listing is “CHMC 2022-0803b Sequence Listing.xml”, the date of the creation of the XML file is Mar. 3, 2025, and the size of the XML file is 77,653 bytes.

### BACKGROUND

Medulloblastoma (MB), a malignant pediatric brain tumor of the posterior fossa, is a highly heterogeneous tumor broadly composed of Sonic Hedgehog (SHH), group 3 (G3), group 4 (G4) and WNT subgroups. Different MB subgroups can arise from diverse cell types or lineages in the developing cerebellum or brainstem and confer distinct treatment responses. The developing cerebellum is composed of distinct neural progenitor populations including progenitor cells in the cerebellar ventricular zone that give rise to GABAergic neuronal lineage cells, and those in the rhombic lip (RL), which generate both granule cell progenitors (GCP) to form the external granule cell layer and glutamatergic populations including unipolar brush cells (UBC). The GCPs, UBC-lineage cells and neural stem or progenitor cells are proposed as potential cells of origin for SHH MB, G4-MB and G3-MB, respectively. The cells of origin of human G3-MB, the most aggressive subgroup associated with the worst prognosis and MYC activation, have not been fully defined. Understanding of MB tumor origins is largely informed by analyses of rodent models. However, the human cerebellum has a 750-fold larger surface area than that of mice, with more primary progenitor populations. Understanding the cellular heterogeneity across human cerebellar development is critical for decoding the developmental origin for G3-MB.

### BRIEF SUMMARY

Disclosed are methods of treating a medulloblastoma (MB) tumor in an individual in need thereof, comprising administering to the individual a composition comprising one or more of a nucleic acid having specific binding to a SOX11 mRNA, a nucleic acid having specific binding to an HNRNPH1 mRNA and/or a nucleic acid having specific binding to a MYC enhancer region. In aspects, the nucleic acid has specific binding to a MYC enhancer region and impedes binding of one or both of a SOX11 protein and a HNRNPH1 protein to the MYC enhancer region. CRISPRi and/or CRISPR methods may be used for delivery of the nucleic acids contemplated herein. Further disclosed are methods for identifying patients in need of such treatment.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Single-cell atlas of early developing human fetal cerebella. a, t-SNE visualization of transcriptionally distinct cell populations from 95,542 single cells in PCW 8-17 human fetal cerebellar tissue. TCPs are circled. Dev., developing; GCP_P, GCP progenitor; UBC_P, UBC progenitor; diff., differentiated. b, Left, neural cell types (red) in the t-SNE map of fetal cerebella. Right, UMAP visualization of neural cell types from fetal cerebella. c, Prediction of neural cell types fate transitions using Monocle 3. d, Heat map with spline curves fitted to differentially expressed genes. Pseudo-temporal trajectories grouped by hierarchical clustering (k=3). e, Proportion of each cell population (top) and the TCP proportion (bottom) at indicated stages. f, Median-scaled In-normalized expression of selected markers for identified fetal cerebellar cell states. Oligoden., oligodendrocyte; intern., interneuron. g, Representative marker gene expression in the indicated cell types. h, A matrix for cross validation with a support vector machine classifier of the identified cell types between the mouse and human fetal cerebella. Endo., endothelial cell; ependy., ependymal cell; pro., progenitor.

FIG. 2. Identification of transitional progenitor intermediate in human fetal cerebella. a, Top, haematoxylin and eosin staining of midsagittal sections of PCW 9-16 human fetal cerebella. Arrows show the RL. Middle and bottom, SOX2/HNRNPH1 and SOX2/SOX11 immunostaining from the boxed areas in the top panels at the corresponding stages. b, The percentage of HNRNPH1+ and SOX11+ cells at indicted stages (n=2 biologically independent samples per stage). VZ, ventricular zone; SVZ, subventricular zone. c, Midsagittal sections of PCW 12 fetal cerebellum immunostaining for HNRNPH1 and SOX11 in the RL transitional zone (TZ) (yellow box). Arrows show the colabelled cells. d, Proportion of TCP cells in the TZ at the indicated stages (n=2 biologically independent samples per stage). e, Immunostaining for SOX11 and HNRNPH1 in the RL (RLSVZ and RLVZ) from the red-boxed area in c at PCW 12. Scale bar, 100 μm. f, Midsagittal sections of PCW 12 fetal cerebellum immunostaining for SOX11 and HNRNPH1 in the TZ. Scale bar, 100 μm. g,h, Representative images (g) and the labelled cell proportions (h) in PCW 12 fetal cerebella stained for HNRNPH1, PAX6 and EOMES. Boxed areas are shown at a higher magnification on the right. Arrows indicated triple-labelling cells. Data are mean and s.e.m. i, The potential transition from the TCP population to the GCP and UBC lineages during the development of human fetal cerebella is shown. The schematic was created using BioRender (https://biorender.com). Scale bars in a,c,f,g, 100 μm.

FIG. 3. scRNA-seq reveals intermediate TCP-like progenitors in aggressive MBs. a, UMAP visualization of G3 (n=9 tumors), G4 (n=13 tumors) and SHH MB (n=4 tumors). The cells are colour-coded for indicated populations identified by representative marker genes. NK, natural killer. b, Heat map of cell populations in human MB subgroups compared to normal human fetal cerebellar cell populations. c, Heat map showing marker gene expression in pseudo-bulk normal fetal TCP and TCP-like tumor cells in SHH, G3 and G4 MBs. d, Cell-state plots of tumor subpopulations in indicated MB subgroups. The positions of cell states (dots) indicate relative meta-module scores, and colour scales represent the count. e, Top, DNA methylation-based subgroup prediction scores for G3 and G4 MBs. Samples G3-7 (BT-309) and G4-1 (BT-325) had a mixture of G3/G4 scores. Bottom, UMAP distributions of G3 and G4 tumor cells from BT-309 and BT-325. PC, photoreceptor cells. f, Heat map of the marker genes in BT-309 and BT-325 tumor cells showing mixed G3 and G4 cell populations.

FIG. 4. TCP regulators mediate long-range enhancer hijacking for G3-MB oncogenesis. a, Heat maps of Cut&Run peaks with indicated antibodies in G3 MB cells and human NSCs showing ±1,000 bp around TSS. b, Signals of H3K4me3, H3K27ac, HNRNPH1 and SOX11 peaks plotted relative to the TSS. c, Enrichment analysis of the genes co-occupied by HNRNPH1 and SOX11 in G3 MB cells. P value was calculated by a one-sided Fisher's exact test adjusted with multiple comparisons. d, Genomic tracks for H3K27ac, SOX11 and HNRNPH1 occupancy at MYC (left) and OTX2 (right) loci of human NSCs and G3 MB cells (MB-004). Astro., human astrocytes. e, Top, Hi-C map of intrachromosomal interaction in G3-MB cells (MB-004) and G4-MB cells (UPN3550). Middle, genomic occupancy. Bottom, pseudo-bulk genome co-accessibility in the MYC locus. Long-distance regulatory elements in human NSCs and G3 MB cells were highlighted. f, Proposed model for the contribution of enhancer hijacking to MYC expression. g, MYC expression after CRISPRi targeting SOX11/HNRNPH1 binding motifs (I, II, II, IV) and an internal motif V relative to control sgRNAs (n=6 independent experiments per motif). h, Relative enrichment of H3K27ac occupancy on the indicated enhancers (Enh1 and Enh2) or promoter (Pro) in MB-004 cells transduced with non-targeting shRNA control (shCtrl), shSOX11 or shHNRNPH1 and in control hNSCs (n=3 independent experiments). In g and h, data are shown as mean±s.e.m., unpaired two-tailed Student's t-test.

FIG. 5. Targeting TCP regulators inhibits aggressive MB growth. a, Kaplan-Meier analysis of overall survival of patients with cerebellar MBs based on the TCP signature score in Cavalli's MB cohorts. log-rank test. b, Left, representative images of human G3 (n=12 tissue samples) and G4 (n=9 samples) MBs stained for HNRNPH1 (left) and SOX11 (right). Scale bars, 100 μm. Right, representative images of HNRNPH1 and SOX11 co-immunostaining (arrows) in G3 MB (n=3 samples). Scale bar, 2 μm. c, Representative images showed the proximity ligation assay of the HNRNPH1-SOX11 association (arrow) in MB-004 cells treated with shCtrl or shHNRNPH1. n=3 independent experiments. Scale bars, 10 μm. d, Representative immunoblots for MYC and HNRNPH1 in MB-004 cells (left) treated with control or shRNAs targeting HNRNPH1 (left) or SOX11 (right). n=3 independent experiments. e, Images (left) and quantification (right) of tumor spheres formed by MB-004 or D425 cells treated with shCtrl, shSOX11 or shHNRNPH1. Scale bars, 200 μm. n=5 independent experiments. f,g, Bioluminescent imaging (f) and survival curves (g) of NSG mice transplanted with MB-004 cells transduced with shCtrl, shSOX11 or shHNRNPH1 at indicated days (n=10 mice per group), log-rank test. h, Quantification of tumor cell proliferation (top, Ki67+) and apoptosis (bottom, CC3+, cleaved caspase 3) in mice grafted with MB-004 cells transduced with shCtrl, shSOX11 or shHNRNPH1. n=5 independent samples/treatment. In e and h, data are shown as the mean±s.e.m. (bars) and individual score (dots); two-tailed unpaired Student's t-test.

### DETAILED DESCRIPTION

### Definitions

Unless otherwise noted, terms are to be understood according to conventional usage by those of ordinary skill in the relevant art. In case of conflict, the present document, including definitions, will control. Exemplary methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the treatment of medulloblastoma.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, “administer,” “administering,” “administration” and the like refers to providing a substance (e.g., an oligonucleotide) to a subject in a manner that is pharmacologically useful (e.g., to treat a condition in the subject).

As used herein, “attenuate,” “attenuating,” “attenuation” and the like refers to reducing or effectively halting. As a non-limiting example, one or more of the treatments herein may reduce or effectively halt the onset or progression of medulloblastoma.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some aspects, complementary nucleotides can base pair in the Watson-Crick manner or in any other manner that allows for the formation of stable duplexes. In some aspects, two nucleic acids may have regions of multiple nucleotides that are complementary with each other to form regions of complementarity, as described herein.

As used herein, “double-stranded oligonucleotide” or “ds oligonucleotide” refers to an oligonucleotide that is substantially in a duplex form. In some aspects, the complementary base-pairing of duplex region(s) of a double-stranded oligonucleotide is formed between antiparallel sequences of nucleotides of covalently separate nucleic acid strands. In some aspects, complementary base-pairing of duplex region(s) of a double-stranded oligonucleotide is formed between antiparallel sequences of nucleotides of nucleic acid strands that are covalently linked. In some aspects, complementary base-pairing of duplex region(s) of a double-stranded oligonucleotide is formed from single nucleic acid strand that is folded (e.g., via a hairpin) to provide complementary antiparallel sequences of nucleotides that base pair together. In some aspects, a double-stranded oligonucleotide comprises two covalently separate nucleic acid strands that are fully duplexed with one another. However, in some aspects, a double-stranded oligonucleotide comprises two covalently separate nucleic acid strands that are partially duplexed (e.g., having overhangs at one or both ends). In some aspects, a double-stranded oligonucleotide comprises antiparallel sequence of nucleotides that are partially complementary, and thus, may have one or more mismatches, which may include internal mismatches or end mismatches.

As used herein, “excipient” refers to a non-therapeutic agent that may be included in a composition, for example, to provide or contribute to a desired consistency or stabilizing effect.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some aspects, the terms refer to humans. In further aspects, the terms may refer to children.

As used herein, “oligonucleotide” refers to a short nucleic acid (e.g., less than about 100 nucleotides in length). An oligonucleotide may be single-stranded (ss) or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA or ss siRNA. In some aspects, a double-stranded oligonucleotide is an RNAi oligonucleotide.

As used herein, “reduced expression” of a gene (e.g., a target gene such as SOX11 (SRY-box transcription factor 11) or HNRNPH1, (Heterogeneous nuclear ribonucleoprotein H1) herein referred to as a Target Gene) refers to a decrease in the amount or level of RNA transcript (e.g., Target Gene mRNA) or protein encoded by the gene and/or a decrease in the amount or level of activity of the gene in a cell, a population of cells, a sample or a subject, when compared to an appropriate reference (e.g., a reference cell, population of cells, sample or subject). For example, the act of contacting a cell with an oligonucleotide herein (e.g., an oligonucleotide comprising an antisense strand having a nucleotide sequence that is complementary to a nucleotide sequence comprising the Target Gene) may result in a decrease in the amount or level of Target Gene mRNA, Target Gene protein and/or Target Gene activity (e.g., via inactivation and/or degradation of Target Gene mRNA by the RNAi pathway) when compared to a cell that is not treated with the double-stranded oligonucleotide. Similarly, and as used herein, “reducing expression” refers to an act that results in reduced expression of a gene (e.g., Target Gene). As used herein, “reduction of Target Gene expression” refers to a decrease in the amount or level of Target Gene mRNA, Target Gene protein and/or Target Gene activity in a cell, a population of cells, a sample or a subject when compared to an appropriate reference (e.g., a reference cell, population of cells, sample, or subject).

As used herein, the term “Target Gene” refers to one or both of SOX11 and HNRNPH1.

As used herein, “region of complementarity” refers to a sequence of nucleotides of a nucleic acid (e.g., a double-stranded oligonucleotide) that is sufficiently complementary to an antiparallel sequence of nucleotides to permit hybridization between the two sequences of nucleotides under appropriate hybridization conditions (e.g., in a phosphate buffer, in a cell, etc.). In some aspects, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a double-stranded oligonucleotide having a sense strand (passenger) and antisense strand (guide), in which the antisense strand or part of the antisense strand is used by the Argonaute 2 (Ago2) endonuclease in the cleavage of a target mRNA (e.g., Target Gene mRNA) or (b) a ss oligonucleotide having a single antisense strand, where that antisense strand (or part of that antisense strand) is used by the Ago2 endonuclease in the cleavage of a target mRNA (e.g., Target Gene mRNA).

As used herein, “strand” refers to a single, contiguous sequence of nucleotides linked together through internucleotide linkages (e.g., phosphodiester linkages or phosphorothioate linkages). In some aspects, a strand has two free ends (e.g., a 5′ end and a 3′ end).

Oligonucleotide Inhibitors of Target Gene and/or MYC Enhancer Region

Applicant has found that SOX11 and HNRNPH1 are upregulated in MB. SOX11 and/or HNRNPH1 binding to the enhancer region of MYC is believed to contribute to the pathology of MB. As such, the present disclosure provides methods for treating an individual having MB, in which the binding of SOX11 and/or HNRNPH1 with a MYC enhancer region is reduced or otherwise inhibited.

For example, in aspects, a nucleic acid that binds to a SOX11 transcript is disclosed, wherein the binding reduces HNRNPH1 binding to an enhancer region of MYC.

In aspects, a nucleic acid that binds to an HNRNPH1 transcript is disclosed, wherein the binding reduces HNRNPH1 binding to an enhancer region of MYC.

In further aspects, a nucleic acid having binding specificity to an enhancer region of MYC is disclosed, wherein the nucleic acid interrupts SOX 11 binding to the MYC enhancer region, HNRNPH1 binding to the MYC enhancer region, or both SOX 11 binding and HNRNPH1 binding to an enhancer region of MYC.

In aspects, the nucleic acid is an RNAi. In aspects, the RNAi binds a SOX11 transcript. In aspects, the RNAi binds an HNRNPH1 transcript.

In aspects, the nucleic acid is an sgRNA. In aspects, the sgRNA binds an MYC enhancer region.

In aspects, the binding of SOX11 and/or HNRNPH1 with a MYC enhancer region is disrupted via administration of an sgRNA using CRISPRi in which a MYC enhancer region site in the genomic DNA is inhibited.

In aspects, the binding of SOX11 and/or HNRNPH1 with a MYC enhancer region is disrupted via administration of an sgRNA using CRISPR in which a MYC enhancer region site in the genomic DNA is disrupted.

The disclosure provides, inter alia, oligonucleotides that inhibit Target Gene expression and/or that inhibit SOX11 and/or HNRNPH1 binding to a MYC enhancer sequence. In some aspects, an oligonucleotide that inhibits Target Gene expression herein is targeted to a Target Gene mRNA. In other aspects, an oligonucleotide is targeted to a MYC enhancer sequence.

### Target Sequences

In some aspects, the oligonucleotide is targeted to a target sequence comprising a Target Gene mRNA. In some aspects, the oligonucleotide is targeted to a target sequence comprising an enhancer region of MYC. The enhancer region of MYC is typically genomic DNA. In aspects, the target sequence is at least 10 nucleotides, or at least 15 nucleotides, or at least 20 nucleotides, or about 25, or about 30 or more than 30 nucleotides of an MYC enhancer regions of Table 2.

In aspects, the oligonucleotide, or a portion, fragment or strand thereof (e.g., an antisense strand or a guide strand of a double-stranded oligonucleotide) binds or anneals to a target sequence. In aspects, the target sequence is a Target Gene mRNA, the bound oligonucleotide inhibiting Target Gene expression or activity. In aspects, the oligonucleotide is targeted to a Target Gene target sequence for the purpose of inhibiting Target Gene expression or activity in vivo. In aspects, the amount or extent of inhibition of Target Gene expression or activity by an oligonucleotide targeted to a Target Gene target sequence correlates with the potency of the oligonucleotide. In aspects, the amount or extent of inhibition of Target Gene expression or activity by an oligonucleotide targeted to a Target Gene target sequence correlates with the amount or extent of therapeutic benefit in a subject or patient having a disease, disorder or condition associated with the expression of Target Gene treated with the oligonucleotide, for example, medulloblastoma.

In aspects, the target sequence is the genomic sequence of an MYC enhancer region, for example a portion of one or more sequences of SEQ ID NOS: 33-38. In aspects, the target sequence is a portion (at least 15 nucleotides, or at least 20 nucleotides, or at least 25 nucleotides of SEQ ID NO: 33. In aspects, the target sequence is a portion (at least 15 nucleotides, or at least 20 nucleotides, or at least 25 nucleotides of SEQ ID NO: 34. In aspects, the target sequence is a portion (at least 15 nucleotides, or at least 20 nucleotides, or at least 25 nucleotides of SEQ ID NO: 35. In aspects, the target sequence is a portion (at least 15 nucleotides, or at least 20 nucleotides, or at least 25 nucleotides of SEQ ID NO: 36. In aspects, the target sequence is a portion (at least 15 nucleotides, or at least 20 nucleotides, or at least 25 nucleotides of SEQ ID NO: 37. In aspects, the target sequence is a portion (at least 15 nucleotides, or at least 20 nucleotides, or at least 25 nucleotides of SEQ ID NO: 38.

In some aspects, the oligonucleotides herein have regions of complementarity to Target Gene mRNA (e.g., within a target sequence of Target Gene mRNA) for purposes of targeting the Target Gene mRNA in cells and inhibiting Target Gene expression or activity. In some aspects, the oligonucleotides herein comprise a targeting sequence (e.g., an antisense strand or a guide strand of a double-stranded oligonucleotide) having a region of complementarity that binds or anneals to a Target Gene target sequence or a MYC enhancer target sequence by complementary (Watson-Crick) base pairing. The targeting sequence or region of complementarity is generally of a suitable length and base content to enable binding or annealing of the oligonucleotide (or a strand thereof) to a Target Gene mRNA for purposes of inhibiting its expression or activity or a MYC enhancer target sequence for purposes of inhibiting binding of SOX11 or HNRNPH1 to the MYC enhancer region. In some aspects, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some aspects, the targeting sequence or region of complementarity is about 12 to about 30 (e.g, 12 to 30, 12 to 22, 15 to 25, 17 to 21, 18 to 27, 19 to 27, or 15 to 30) nucleotides in length. In some aspects, the targeting sequence or region of complementarity is about 12, 13, 14, 15, 16, 17, 18, 19, 20, 21, 22, 23, 24, 25, 26, 27, 28, 29 or 30 nucleotides in length. In some aspects, the targeting sequence or region of complementarity is 18 nucleotides in length. In some aspects, the targeting sequence or region of complementarity is 19 nucleotides in length. In some aspects, the targeting sequence or region of complementarity is 20 nucleotides in length. In some aspects, the targeting sequence or region of complementarity is 21 nucleotides in length. In some aspects, the targeting sequence or region of complementarity is 22 nucleotides in length. In some aspects, the targeting sequence or region of complementarity is 23 nucleotides in length. In some aspects, the targeting sequence or region of complementarity is 24 nucleotides in length.

In some aspects, the disclosure provides an RNAi oligonucleotide for reducing Target Gene expression or activity, the oligonucleotide comprising a sense strand and an antisense strand, wherein the antisense strand comprises a region of complementarity to a Target Gene mRNA target sequence of any one of SEQ ID NOs 1-8. In aspects, the region of complementarity is at least 15 contiguous nucleotides in length. In one aspect, the Target Gene mRNA sequence is any one of SEQ ID NOs 1-8.

In some aspects, an oligonucleotide herein comprises a targeting sequence or a region of complementarity (e.g, an antisense strand or a guide strand of a double-stranded oligonucleotide) that is fully complementary to a Target Gene target sequence. In some aspects, the targeting sequence or region of complementarity is partially complementary to a Target Gene target sequence. In some aspects, the oligonucleotide comprises a targeting sequence or region of complementarity that is fully complementary to a sequence of any one of SEQ ID NOs: 39 and/or 40. In some aspects, the oligonucleotide comprises a targeting sequence or region of complementarity that is partially complementary to a sequence of any one of SEQ ID NOs: 39 and/or 40.

A variety of oligonucleotide types and/or structures are useful for targeting a sequence in the methods herein including, but not limited to, RNAi oligonucleotides, antisense oligonucleotides, miRNAs, etc. Any of the oligonucleotide types described herein or elsewhere are contemplated for use as a framework to incorporate an mRNA targeting sequence herein for the purposes of inhibiting Target Gene expression or activity.

Other oligonucleotide designs for use with the compositions and methods herein include: 16-mer siRNAs, shRNAs (e.g., having 19 bp or shorter stems), blunt siRNAs (e.g., of 19 bps in length), asymmetrical siRNAs (aiRNA), asymmetric shorterduplex siRNA, fork siRNAs, ss siRNAs, dumbbell-shaped circular siRNAs, and small internally segmented interfering RNA. Further non-limiting examples of oligonucleotide structures that may be used in some aspects to reduce or inhibit the expression of Target Gene are microRNA (miRNA), short hairpin RNA (shRNA) and short siRNA (see, e.g., US Patent Application Publication No. 2009/0099115).

In some aspects, an oligonucleotide for reducing or inhibiting Target Gene expression or activity herein is single-stranded (ss). Such structures may include but are not limited to ss RNAi molecules. In some aspects, an inhibitory oligonucleotide herein are antisense oligonucleotides (ASOs). An antisense oligonucleotide is a ss oligonucleotide that has a nucleobase sequence which, when written or depicted in the 5′ to 3′ direction, comprises the reverse complement of a targeted segment of a particular nucleic acid and is suitably modified (e.g., as a gapmer) so as to induce RNaseH-mediated cleavage of its target RNA in cells or (e.g., as a mixmer) so as to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes. In aspects, oligonucleotides, such as double-stranded oligonucleotides, for targeting Target Gene mRNA and inhibiting Target Gene expression or activity (e.g., via the RNAi pathway) are disclosed. In aspects, the oligonucleotides comprise a sense strand (also referred to herein as a passenger strand) and an antisense strand (also referred to herein as a guide strand). In some aspects, the sense strand and antisense strand are separate strands and are not covalently linked. In some aspects, the sense strand and antisense strand are covalently linked.

It should be appreciated that, in some aspects, sequences presented in the Sequence Listing may be referred to in describing the structure of an oligonucleotide (e.g., a double-stranded oligonucleotide) or other nucleic acid. In such aspects, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified antinucleotide linkages and/or one or more other modification when compared with the specified sequence while retaining essentially same or similar complementary properties as the specified sequence.

The oligonucleotide may comprise one or more modification such as a polyA tail, 5′ cap analog (e.g., Anti Reverse Cap Analog (ARCA) or m7G(5′)ppp(5′)G (mCAP)), modified 5′ or 3′ untranslated region (UTR), use of modified bases (such as Pseudo-UTP, 2-Thio-UTP, 5-Methylcytidine-5′-Triphosphate (5-Methyl-CTP) or N6-Methyl-ATP), or treatment with phosphatase to remove 5′ terminal phosphates. These and other modifications are known in the art, and new modifications of RNAs are regularly being developed.

### CRISPR

In further aspects, the disclosed methods may employ CRISPRi (CRISPR interference) and/or and CRISPRa (CRISPR activation) technology, for example as described in U.S. Pat. No. 11,130,955. In some aspects, the presently disclosed technologies utilize catalytically inactivated (i.e., nuclease-deactivated) CRISPR endonucleases that have been mutated to no longer generate double DNA stranded breaks, but which are still able to bind to DNA target sites through their corresponding guide RNAs.

In some aspects, the CRISPRi methods utilize dCRISPR enzymes to occupy target DNA sequences necessary for transcription, thus blocking the transcription of the targeted gene. In other aspects, the CRISPRi methods of the present disclosure utilize dCRISPR enzymes translationally fused, or otherwise tethered to one or more transcriptional repression domains, or alternatively utilize modified guide RNAs capable of recruiting transcriptional repression domains to the target site (e.g., tethered via aptamers, as discussed below).

In some aspects, the CRISPRa methods employ dCRISPR enzymes (translationally fused or otherwise tethered to different transcriptional activation domains, which can be directed to promoter regions by guide RNAs. Catalytically inactivated CRISPR enzymes are referred to as “dead CRISPR”, or “dCRISPR” enzymes. The “dead” modifier may also be used in reference to specific CRISPR enzymes, such as dead Cas9 (dCas9), or dead Cpf1 (dCpf1). In other aspects, the CRISPRa methods utilize modified guide RNAs that recruit additional transcriptional activation domains to upregulate expression of the target gene (e.g., tethered via aptamers).

In aspects, the disclosed methods employ one or more sgRNA or gRNA. sgRNA and gRNA are described in, for example, U.S. Patent Application 2020/0040061. The sgRNA or gRNA can be introduced into the cell for treatment of MB, for example via interfering or removing a binding site for SOX11 and/or HNRNPH1. In aspects, one or more sgRNA or gRNA comprise an RNA sequence corresponding to a CRISPRi MYC Enhancer sequence of Table 1. In aspects, the one or more sgRNA or gRNA comprise one or more sequences corresponding to (complementary to) SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20. A knock-down or knock-out strategy can involve disrupting a binding site in a MYC enhancer region by introducing random insertions or deletions (indels) within or near the MYC enhancer region which serves as a binding site for SOX11 and/or HNRNPH1. This can be achieved by inducing one single stranded break or double stranded break in the target sequence with one or more CRISPR endonucleases and a gRNA (e.g., crRNA+tracrRNA, or sgRNA), or two or more single stranded breaks or double stranded breaks in the target sequence with two or more CRISPR endonucleases and two or more sgRNAs.

Alternatively, a knock-down or knock-out strategy can also involve deletion of one or more segments within or near the target sequence. This deletion strategy employs at least a pair of gRNAs (e.g., crRNA+tracrRNA, or sgRNA) capable of binding to two different sites within or near the target sequence and one or more CRISPR endonucleases. The CRISPR endonucleases, configured with the two gRNAs, induce two double stranded breaks at the desired locations. After cleavage, the two ends, regardless of whether blunt or with overhangs, can be joined by NHEJ, leading to the deletion of the intervening fragment. NHEJ repair pathways can lead to insertions, deletions or mutations at the joints.

A single-molecule guide RNA (sgRNA) in a Type II system can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. The sgRNA can comprise a 20 nucleotide spacer sequence at the 5′ end of the sgRNA sequence. The sgRNA can comprise a less than a 20 nucleotide spacer sequence at the 5′ end of the sgRNA sequence. The sgRNA can comprise a more than 20 nucleotide spacer sequence at the 5′ end of the sgRNA sequence. The sgRNA can comprise a variable length spacer sequence with 17-30 nucleotides at the 5′ end of the sgRNA sequence. The sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil at the 3′ end of the sgRNA sequence. The sgRNA can comprise 5 uracil at the 3′ end of the sgRNA sequence. The sgRNA can comprise 6 uracil at the 3′ end of the sgRNA sequence. The sgRNA can comprise 7 uracil at the 3′ end of the sgRNA sequence. The sgRNA can comprise 8 uracil at the 3′ end of the sgRNA sequence. The sgRNA can be unmodified or modified. For example, modified sgRNAs can comprise one or more 2′-O-methyl phosphorothioate nucleotides. The guide RNAs can be chemically linked to one or more moieties or conjugates that enhance the activity, cellular distribution, or cellular uptake of the oligonucleotide. Such moieties comprise, but are not limited to, lipid moieties such as a cholesterol moiety; cholic acid; a thioether, e.g., hexyl-S-tritylthiol; a thiocholesterol; an aliphatic chain, e.g., dodecandiol or undecyl residues; a phospholipid, e.g., di-hexadecyl-rac-glycerol or triethylammonium 1,2-di-O-hexadecyl-rac-glycero-3-H-phosphonate; a polyamine or a polyethylene glycol chain; adamantane acetic acid; a palmityl moiety; or an octadecylamine or hexylamino-carbonyl-t oxycholesterol moiety.

### Vectors

In aspects, the disclosed methods may employ a vector. As used herein, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

In aspects, the vector is a lentivirus vector particle comprising a lentiviral genome which encodes at least one guide RNA sequence that is complementary to a first DNA sequence in a host cell genome. Lentiviruses are a subclass of Retroviruses that resemble γ-retroviruses (γ-RV) in their ability to stably integrate into the target cell genome, resulting in persistent expression of the gene of interest. Lentiviral vectors are described in, for example, U.S. Pat. No. 11,203,768.

It will be appreciated by those skilled in the art that the design of the expression vector can depend on such factors as the choice of the host cell to be transformed, the level of expression desired, etc.

### Pharmaceutical Compositions

In other aspects, the disclosure provides a pharmaceutical composition comprising a therapeutic nucleic acid as described herein, and a pharmaceutically acceptable carrier, delivery agent or excipient.

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some aspects, an oligonucleotide is formulated in buffer solutions such as phosphate buffered saline solutions, liposomes, micellar structures and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine, can be used.

Accordingly, in some aspects, a formulation comprises a lipid nanoparticle. In some aspects, an excipient comprises a liposome, a lipid, a lipid complex, a microsphere, a microparticle, a nanosphere or a nanoparticle, or may be otherwise formulated for administration to the cells, tissues, organs, or body of a subject in need thereof.

In some aspects, the formulations herein comprise an excipient. In some aspects, an excipient confers to a composition improved stability, improved absorption, improved solubility and/or therapeutic enhancement of the active ingredient. In some aspects, an excipient is a buffering agent (e.g., sodium citrate, sodium phosphate, a tris base, or sodium hydroxide) or a vehicle (e.g., a buffered solution, petrolatum, dimethyl sulfoxide or mineral oil). In some aspects, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g. mannitol, lactose, polyethylene glycol or polyvinylpyrrolidone) or a collapse temperature modifier (e.g., dextran or gelatin).

In some aspects, a composition may contain at least about 0.1% of the therapeutic nucleic acid or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though several aspects are directed to central nervous system-targeted delivery of any of the therapeutic nucleic acids herein, targeting of other tissues is also contemplated. For example, the target cell may be present in the CNS, such as the brain or spinal cord. In some aspects, the cell is a brain cell. In some aspects, the cell is a frontal cortical cell or a frontal temporal lobe cell. In some aspects, the target cell is a cell of the thalamus, hippocampus, striatum, retina, or spinal cord.

### Reducing Expression in a Cell

In another aspect, the disclosure provides a method for reducing Target Gene expression or activity and/or inhibiting SOX11 and/or HNRNPH1 binding to a MYC enhancer region in a cell, a population of cells or a subject. In aspects, the method comprises the step of: i. contacting the cell or the population of cells with a therapeutic nucleic acid (sgRNA, RNAi, or the like) or pharmaceutical composition described herein; or ii. administering to the subject a therapeutic nucleic acid or pharmaceutical composition described herein. In some aspects, the subject has medulloblastoma. In some aspects, the disease, disorder, or condition associated with Target Gene expression or activity is medulloblastoma.

The disclosure provides methods for contacting or delivering to a cell or population of cells an effective amount any of the therapeutic nucleic acids (e.g., a double-stranded oligonucleotide, RNAi, sgRNA, or the like) herein for purposes of reducing Target Gene expression and/or for the purpose of inhibiting SOX11 and/or HNRNPH1 binding to a MYC enhancer region. In some aspects, a reduction of Target Gene expression is determined by measuring a reduction in the amount or level of Target Gene mRNA, Target Gene protein, or Target Gene activity in a cell. The methods can include the steps described herein, and these maybe be, but not necessarily, carried out in the sequence as described. Other sequences, however, also are conceivable. Moreover, individual or multiple steps may be carried out either in parallel and/or overlapping in time and/or individually or in multiply repeated steps. Furthermore, the methods may include additional, unspecified steps.

Methods herein are useful in any appropriate cell type. In some aspects, a cell is a cell of the brain. In aspects, the cell is a primary cell obtained from a subject. In aspects, a cell to which the therapeutic nucleic acid is delivered is ex vivo or in vitro (i.e., can be delivered to a cell in culture or to an organism in which the cell resides). In aspects, it is desirable to target the therapeutic nucleic acid of the disclosure to one or more cells or one or more organs. Such a strategy can help to avoid undesirable effects in other organs or avoid undue loss of the therapeutic nucleic acid to cells, tissue or organs that would not benefit from the therapeutic nucleic acid. Accordingly, in some aspects, oligonucleotides disclosed herein are modified to facilitate targeting and/or delivery to a tissue, cell or organ (e.g., to facilitate delivery of the oligonucleotide to the brain). In some aspects, an oligonucleotide comprises at least one nucleotide (e.g., 1, 2, 3, 4, 5, 6 or more nucleotides) conjugated to one or more targeting ligand(s).

In some aspects, the targeting ligand comprises a carbohydrate, amino sugar, cholesterol, peptide, polypeptide, protein or part of a protein (e.g., an antibody or antibody fragment), or lipid. In some aspects, the targeting ligand is an aptamer. For example, a targeting ligand may be an RGD peptide that is used to target tumor vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In certain aspects, the targeting ligand is one or more GalNAc moieties.

Appropriate methods or chemistry (e.g., click chemistry) can be used to link a targeting ligand to a nucleotide. In some aspects, a targeting ligand is conjugated to a nucleotide using a click linker. In some aspects, an acetal-based linker is used to conjugate a targeting ligand to a nucleotide of any one of the oligonucleotides described herein. Acetal-based linkers are disclosed, for example, in Inti. Patent Application Publication No. WO 2016/100401. Various appropriate methods or chemistry synthetic techniques (e.g., click chemistry) can be used to link a targeting ligand to a nucleotide. In some aspects, a targeting ligand is conjugated to a nucleotide using a click linker. In some aspects, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some aspects, the linker is a labile linker. In aspects, the linker is a stable linker.

In aspects, the therapeutic nucleic acids herein are delivered to a cell or population of cells using a nucleic acid delivery method known in the art including, but not limited to, injection of a solution containing the oligonucleotide, bombardment by particles covered by the therapeutic nucleic acid, exposing the cell or population of cells to a solution containing the therapeutic nucleic acid, or electroporation of cell membranes in the presence of the therapeutic nucleic acid. Other methods known in the art for delivering therapeutic nucleic acids to cells may be used, such as lipid-mediated carrier transport, chemical-mediated transport, and cationic liposome transfection such as calcium phosphate, and others.

In some aspects, contacting or delivering an the therapeutic nucleic acids (e.g., a double-stranded oligonucleotide, RNAi, sgRNA or the like) herein to a cell or a population of cells results in a reduction in Target Gene expression and/or SOX11 and/or HNRNPH1 binding to a MYC enhancer region. In some aspects, the reduction in Target Gene expression or activity or binding of SOX11 and/or HNRNPH1 to a MYC enhancer region is relative to a control amount or level in a cell or population of cells not contacted with the therapeutic nucleic acid or contacted with a control oligonucleotide. In some aspects, the reduction in Target Gene expression or activity or SOX11 and/or HNRNPH1 binding is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or pretreatment level. In some aspects, the control amount or level is an amount or level in a cell or population of cells that has not been contacted with the therapeutic nucleic acid. In some aspects, the effect of delivery of the therapeutic nucleic acids to a cell or population of cells according to a method herein is assessed after any finite period or amount of time (e.g., minutes, hours, days, weeks, months). For example, in some aspects, expression or activity is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotide to the cell or population of cells. In some aspects, expression or activity is determined in a cell or population of cells at least about 1 month, about 2 months, about 3 months, about 4 months, about 5 months, or about 6 months or more after contacting or delivering the therapeutic nucleic acid to the cell or population of cells.

In some aspects, the therapeutic nucleic acid is delivered in the form of a transgene that is engineered to express in a cell the therapeutic nucleic acid. In some aspects, the therapeutic nucleic acid is delivered using a transgene engineered to express any of the therapeutic nucleic acid disclosed herein. Transgenes may be delivered using viral vectors (e.g., adenovirus, retrovirus, vaccinia virus, poxvirus, adenoassociated virus or herpes simplex virus) or non-viral vectors (e.g., plasmids or synthetic mRNAs). In some aspects, transgenes can be injected directly to a subject.

The disclosure also provides oligonucleotides for use, or adaptable for use, to treat a subject that would benefit from reducing Target Gene expression and/or SOX11 and/or HNRNPH1 binding to a MYC enhancer region, for example, an individual having medulloblastoma. In some aspects, the disclosure provides therapeutic nucleic acids for use, or adapted for use, to treat a subject having medulloblastoma. The disclosure also provides therapeutic nucleic acids for use, or adaptable for use, in the manufacture of a medicament or pharmaceutical composition for treating medulloblastoma. In some aspects, the therapeutic nucleic acids for use, or adaptable for use, target Target Gene mRNA and reduce Target Gene expression (e.g., via the RNAi pathway) and/or reduce SOX11 and/or HNRNPH1 binding to a MYC enhancer region.

In addition, in some aspects of the methods herein, a subject having medulloblastoma or is predisposed to the same is selected for treatment with a therapeutic nucleic acids herein. In some aspects, the method comprises selecting an individual having a marker (e.g., a biomarker) such as elevated SOX11 and/or elevated HNRNPH1 expression or activity, for medulloblastoma, or predisposed to the same. Likewise, and as detailed below, some aspects of the methods provided by the disclosure include steps such as measuring or obtaining a baseline value for a marker of Target Gene expression or activity, and then comparing such obtained value to one or more other baseline values or values obtained after the subject is administered the oligonucleotide to assess the effectiveness of treatment.

### Methods of Treatment

In some aspects, the disclosure provides a method for treating a subject having a disease, disorder or condition associated with expression of one or more of the Target Genes, the method comprising administering to the subject a therapeutically effective amount of the inhibitory oligonucleotide as disclosed herein, or pharmaceutical composition thereof, thereby treating the subject.

In other aspects, the disclosure provides a method of delivering a therapeutic nucleic acid to a subject, the method comprising administering a pharmaceutical composition described herein to the subject. In particular, the compounds and compositions of the disclosure may be administered locally to brain tissue of the subject, such as brain tissue determined to be responsible for the underlying pathology in the subject. Local administration to the brain generally includes any method suitable for delivery of an inhibitory oligonucleotide or compositions containing the same to brain cells (e.g., neural cells), such that at least a portion of cells of a selected, synaptically connected cell population is contacted with the composition. A therapeutic nucleic acid may be delivered to any cells of the CNS, including neurons, glia, or both. Generally, a therapeutic nucleic acid is delivered to cells of the CNS, including, e.g., cells of the spinal cord, brainstem (medulla, pons, and midbrain), cerebellum, diencephalon (e.g., thalamus and hypothalamus), telencephalon (corpus striatum, cerebral cortex (e.g., cortical regions in the occipital, temporal, parietal, or frontal lobes), or combinations thereof, or any suitable subpopulation of cells therein. Further sites for delivery include the red nucleus, amygdala, entorhinal cortex, and neurons in ventrolateral or anterior nuclei of the thalamus.

In aspects, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intraarterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g, the brain).

In one example, the method of the disclosure includes intracerebral or intracerebroventricular administration through stereotaxic injections. However, other known delivery methods may also be adapted in accordance with the disclosure. For example, for a more widespread distribution of the composition across the CNS, it may be injected into the cerebrospinal fluid, e.g., by lumbar puncture. To direct the composition to the peripheral nervous system (PNS), it may be injected into the spinal cord, one or more peripheral ganglia, or under the skin (subcutaneously or intramuscularly) of the body part of interest. In certain situations, the composition can be administered via an intravascular approach. For example, the composition can be administered intra-arterially (carotid) in situations where the blood-brain barrier is disturbed or not disturbed. Moreover, for more global delivery, the composition can be administered during the “opening” of the blood-brain barrier achieved by infusion of hypertonic solutions including mannitol. Exemplary methods are described in, for example, WO2023141507 and WO2021022208.

As a non-limiting set of examples, the therapeutic nucleic acids herein can be administered quarterly (once every three months), bi-monthly (once every two months), monthly or weekly. For example, the therapeutic nucleic acids may be administered every week or at intervals of two, or three weeks. Alternatively, the therapeutic nucleic acids may be administered daily. In some aspects, a subject is administered one or more loading doses of the therapeutic nucleic acid followed by one or more maintenance doses of the therapeutic nucleic acid.

In another aspect, the disclosure provides a method for treating a subject having medulloblastoma, the method comprising administering to the subject a therapeutically effective amount of an RNAi oligonucleotide comprising a sense strand and an antisense strand, wherein the sense strand and the antisense strand form a duplex region, wherein the antisense strand comprises a region of complementarity to a Target Gene mRNA target sequence of SEQ ID NOs: 39 and/or SEQ ID NO: 40, and wherein the region of complementarity is at least 15 contiguous nucleotides in length.

The disclosure also provides methods of treating a subject having, suspected of having, or at risk of developing medulloblastoma with an oligonucleotide herein. In some aspects, the disclosure provides methods of treating or attenuating the onset or progression of medulloblastoma using the oligonucleotides herein. In other aspects, the disclosure provides methods to achieve one or more therapeutic benefits in a subject having medulloblastoma using the oligonucleotides herein. In some aspects of the methods herein, the subject is treated by administering a therapeutically effective amount of any one or more of the oligonucleotides herein. In some aspects, the subject is treated therapeutically. In some aspects, the subject is treated prophylactically.

In some aspects of the methods herein, an oligonucleotide herein, or a pharmaceutical composition comprising the oligonucleotide, is administered to a subject having medulloblastoma such that Target Gene expression is reduced in the subject, thereby treating the subject. In some aspects, an amount or level of Target Gene mRNA is reduced in the subject. In some aspects, an amount or level of Target Gene protein is reduced in the subject. In some aspects, an amount or level of mRNA of the Target Gene is reduced in the subject. In some aspects, an amount or level of Target Gene activity is reduced in the subject.

In some aspects of the methods herein, an oligonucleotide herein, or a pharmaceutical composition comprising the oligonucleotide, is administered to a subject having medulloblastoma such that Target Gene expression or activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to Target Gene expression or activity prior to administration of the oligonucleotide or pharmaceutical composition. In some aspects, Target Gene expression or activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to Target Gene expression or activity in a subject (e.g., a reference or control subject) not receiving the oligonucleotide or pharmaceutical composition or receiving a control oligonucleotide, pharmaceutical composition or treatment.

In some aspects of the methods herein, an oligonucleotide herein, or a pharmaceutical composition comprising the oligonucleotide, is administered to a subject having medulloblastoma such that an amount or level of Target Gene mRNA or activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of Target Gene mRNA or activity prior to administration of the oligonucleotide or pharmaceutical composition. In some aspects, an amount or level of Target Gene mRNA or activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of Target Gene mRNA or activity in a subject (e.g., a reference or control subject) not receiving the oligonucleotide or pharmaceutical composition or receiving a control oligonucleotide, pharmaceutical composition or treatment.

Methods described herein are typically involve administering to a subject a therapeutically effective amount of an oligonucleotide herein, that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some aspects, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Methods of Diagnosis/Prognosis

In aspects, a method for distinguishing G3 MB and G4 MB is described. For example, in aspects the method comprises detecting expression of one or more of HNRNPH1, PPP1R14A, SOX11, and combinations thereof. In aspects, the method comprises detecting expression of one or both of HNRNPH1 and PPP1R14A, wherein a higher level of one or both of HNRNPH1 and PPP1R14A expression, as compared to a control value, is indicative of G3 MBs. The control value will be understood by one of ordinary skill in the art and may include, for example, the value observed in the average healthy individual not diagnosed with either G3 MB and/or G4 MB. The control value may be age matched, sex matched, or both. In aspects, the method comprises detecting expression of SOX11, wherein a higher level of SOX11, as compared to a control value, is indicative of G4 MB. In aspects, the method comprises determining a prognosis of an individual diagnosed with G3-MB, comprising determining a transitional cerebellar progenitors (TCP)-like cell score, wherein an individual with a high TCP score has a worse prognosis as compared to an individual having a low TCP score. In further aspects, a method for identifying an individual likely to develop MB is disclosed, the method comprising detecting an intermediate progenitor population in fetal cerebellum of the individual. Methods of detection and quantification of gene expression, as well as comparison of such levels to a control value, are understood by one of ordinary skill in the art.

In aspects, a method for distinguishing G3 MB and G4 MB is disclosed, the method comprising detecting expression of one or more of HNRNPH1, PPP1R14A, SOX11, and combinations thereof.

In aspects, the method comprises detecting one or both of HNRNPH1 and PPP1R14A, wherein a higher level of one or both of HNRNPH1 and PPP1R14A, as compared to a control value, is indicative of a diagnosis of G3 MB.

In aspects, the method comprises detecting expression of SOX11, wherein a higher level of SOX11, as compared to a control value, is indicative of a diagnosis of G4 MB.

In aspects, a method of determining a prognosis of an individual diagnosed with G3-MB is disclosed, the method comprising determining a transitional cerebellar progenitors (TCP)-like cell score, wherein an individual with a high TCP score has a worse prognosis as compared to an individual having a low TCP score. In aspects, a method for identifying an individual likely to develop MB is disclosed, the method comprising detecting an intermediate progenitor population in fetal cerebellum of the individual.

### Kits

In some aspects, the disclosure provides a kit comprising an oligonucleotide herein, and instructions for use. In some aspects, the kit comprises an oligonucleotide herein, and a package insert containing instructions for use of the kit and/or any component thereof. In some aspects, the kit comprises, in a suitable container, an oligonucleotide herein, one or more controls, and various buffers, reagents, enzymes and other standard ingredients well known in the art. In some aspects, the container comprises at least one vial, well, test tube, flask, bottle, syringe or other container means, into which the oligonucleotide is placed, and in some instances, suitably aliquoted. In some aspects where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings.

In some aspects, a kit comprises an oligonucleotide herein, and a pharmaceutically acceptable carrier, or a pharmaceutical composition comprising the oligonucleotide and instructions for treating or delaying progression of medulloblastoma in a subject in need thereof.

The following non-limiting examples are provided to further illustrate aspects of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific aspects that are disclosed and still obtain a like or similar result without departing from the spirit and scope of the invention.

### Example 1

Medulloblastoma (MB) is the most common malignant childhood brain tumor yet the origin of the most aggressive subgroup-3 form remains elusive, impeding development of effective targeted treatments. Previous analyses of mouse cerebella have not fully defined the compositional heterogeneity of MBs. Here Applicant undertook single-cell profiling of freshly isolated human fetal cerebella to establish a reference map delineating hierarchical cellular states in MBs. Applicant identified a unique transitional cerebellar progenitor connecting neural stem cells to neuronal lineages in developing fetal cerebella. Intersectional analysis revealed that the transitional progenitors were enriched in aggressive MB subgroups, including group 3 and metastatic tumors. Single-cell multi-omics revealed underlying regulatory networks in the transitional progenitor populations, including transcriptional determinants HNRNPH1 and SOX11, which are correlated with clinical prognosis in group 3 MBs. Genomic and Hi-C profiling identified de novo long-range chromatin loops juxtaposing HNRNPH1/SOX11-targeted super-enhancers to cis-regulatory elements of MYC, an oncogenic driver for group 3 MBs. Targeting the transitional progenitor regulators inhibited MYC expression and MYC-driven group 3 MB growth. Integrated single-cell atlases of human fetal cerebella and MBs show potential cell populations predisposed to transformation and regulatory circuitries underlying tumor cell states and oncogenesis, highlighting hitherto unrecognized transitional progenitor intermediates predictive of disease prognosis and potential therapeutic vulnerabilities.

Applicant carried out single-cell transcriptomics profiling of whole cells from freshly isolated human fetal cerebella to define cellular hierarchy, transitional cell states and their lineage trajectories during early cerebellar development. These data were compared to single-cell transcriptomes of MB subgroups to explain their developmental programs and identified a previously unrecognized transitional intermediate progenitor population in the fetal cerebellum as a potential cell of origin for aggressive MBs such as G3 tumors. Integrative single-cell multi-omics with three-dimensional-(3D-)genome architecture analyses further revealed unique tumor-driver networks and enhancer-hijacking events correlated with MYC activation, pointing to potential therapeutic avenues.

### Human Transitional Cerebellar Progenitors

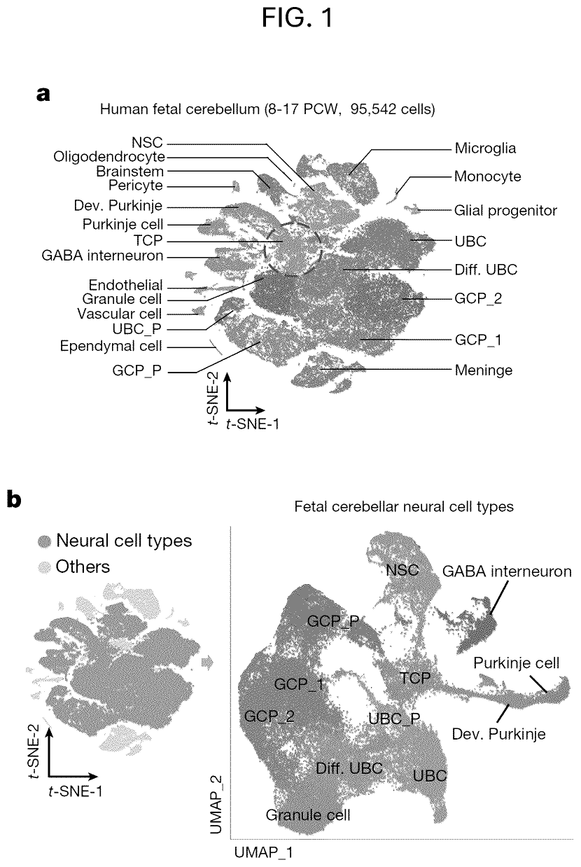

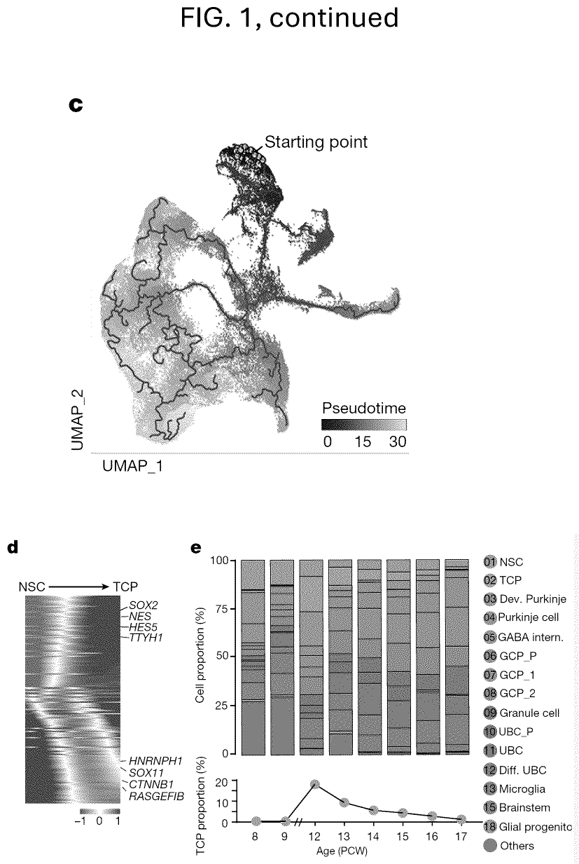

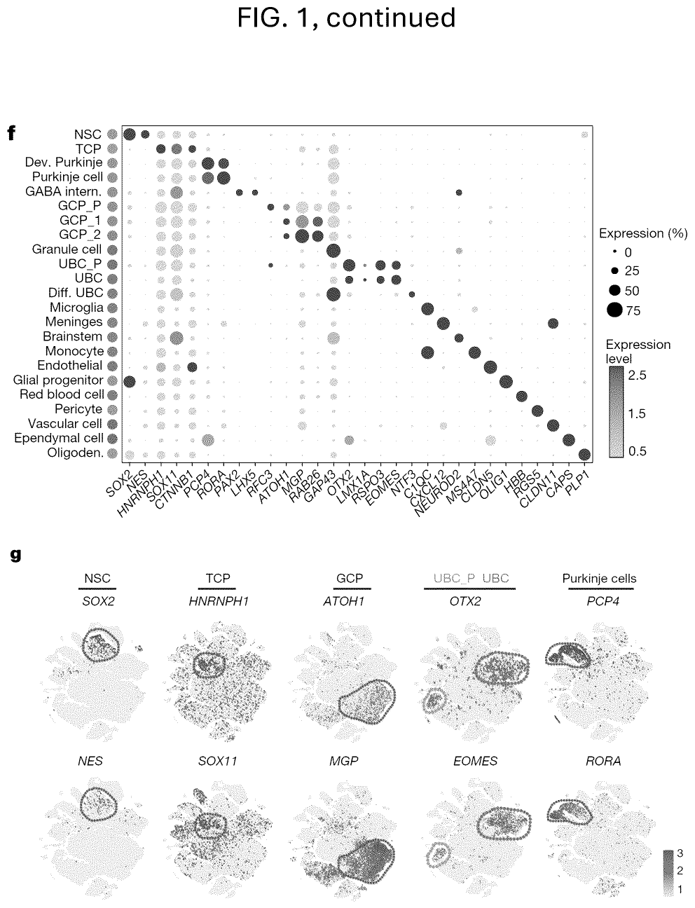

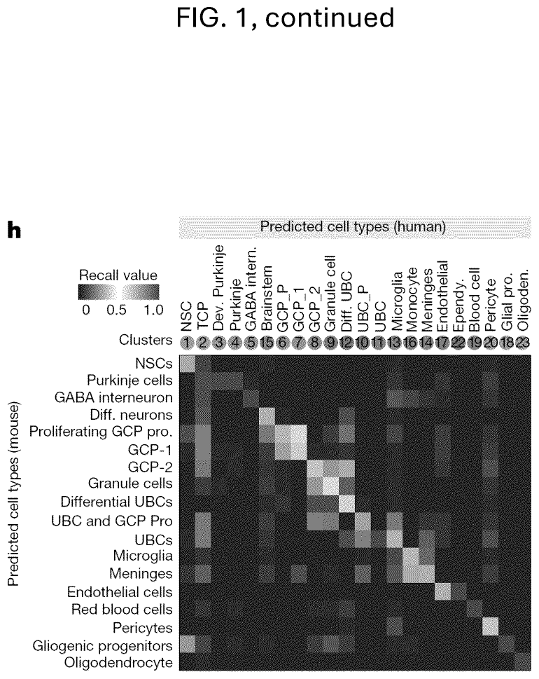

Fresh cerebellar tissues were isolated from aborted fetuses from post conception weeks (PCW) 8 to 17 and profiled roughly 95,542 cells by single-cell RNA-sequencing (scRNA-seq) after quality control and doublet removal. Higher numbers of genes or read counts per cell were obtained in the scRNA-seq data than those from single-nucleotide RNA-seq (snRNA-seq) experiments. Unsupervised clustering of individual cell transcriptomes visualized by t-SNE (t-distributed stochastic neighbor embedding) or uniform manifold approximation and projection (UMAP) identified 23 clusters (FIG. 1, a). Major cell types in human fetal cerebella were annotated by interrogating expression patterns of canonical markers for different cell lineages; the frequencies of cell types varied over time postconception (FIG. 1, a).

To focus on neural cell types, the presumed origin of MB, cell lineage trajectories were investigated using Monocle analysis. The neural stem cell (NSC) population was predicted on the basis of the stemness score as the starting point and showed a trajectory through transitional cerebellar progenitors (TCPs) to the three main neuronal lineage branches, GCPs, UBCs and Purkinje cells (FIG. 1, b,c). STREAM confirmed this hierarchy. To define expression dynamics along the trajectory, Slingshot was used and found successional gene expression from NSCs to TCPs (FIG. 1, d). From PCW 8 to 17, there was an increase in the proportion of Purkinje, then GCP and UBC lineage cells (FIG. 1, e), consistent with known dynamics. TCP cells were abundant at PCW 12 and 13, but their frequency gradually decreased after PCW 14 (FIG. 1, e). Subclustering indicated that a TCP population also expressed cell-cycle genes in G1/S and G2/M phases and proliferative marker Ki67, suggesting that they are in a mitotic state. The TCP population was enriched in expression of HNRNPH1 and SOX11 and distinct from previously defined NSC, GCP and UBC lineage cells (FIG. 1, f,g), and the gene signature of TCP cells partially overlapped with that of RL cells. The populations identified were further compared to published reference profiles of developing mouse cerebella using an algorithm for two-group classification and linked inference of genomic experimental relationships analysis. Most human fetal cerebellar populations shared similarity with mouse counterparts; however, molecular features of the TCP population were not enriched in any known mouse cerebellar cell population (FIG. 1, h).

### In Vivo Validation of TCP Progenitors

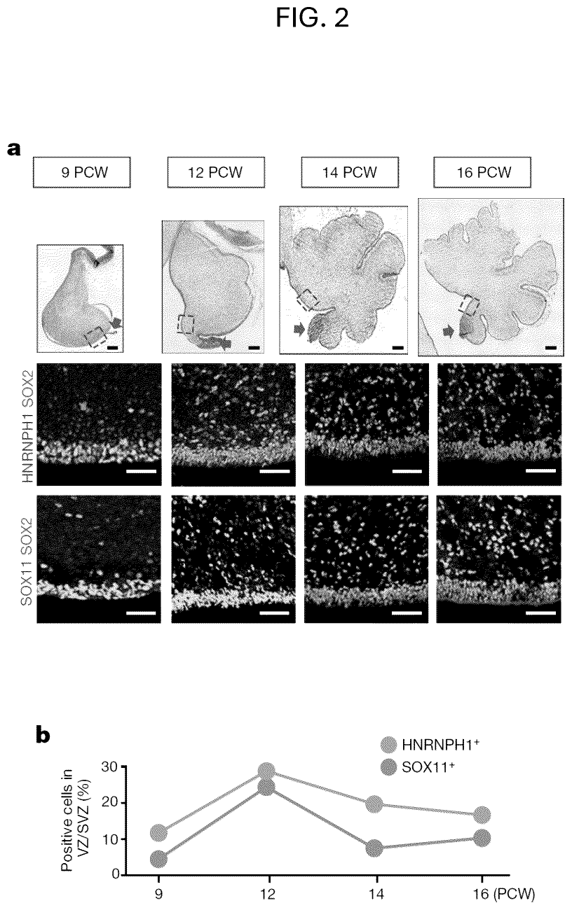

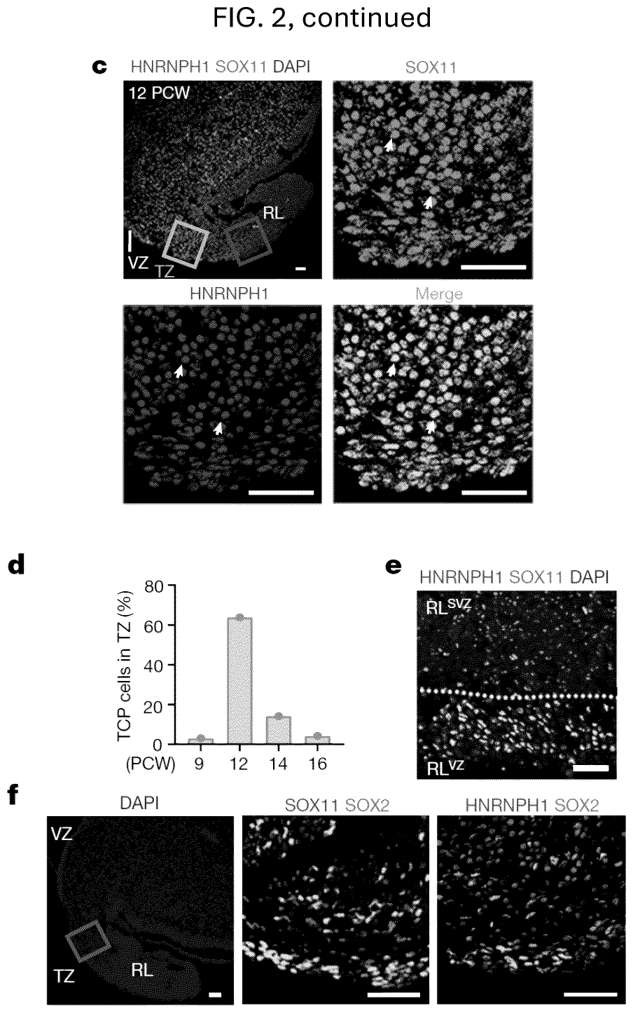

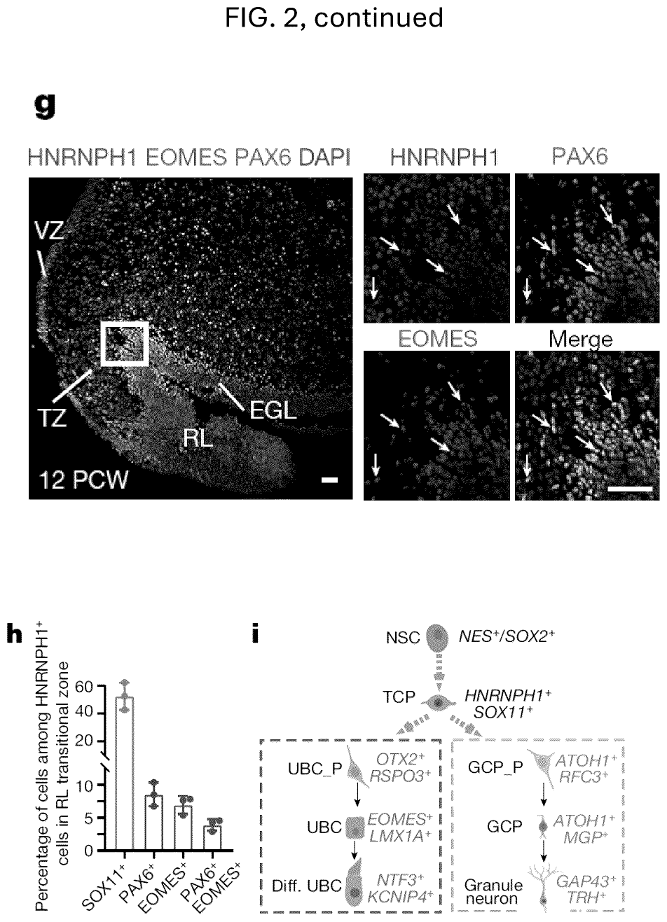

The expression of HNRNPH1 and SOX11 were examined, the most highly enriched TCP signature markers, in human fetal cerebella. HNRNPH1+ and SOX11+ TCP cells were increased in regions adjacent to the NSC (SOX2+) niche in the ventricular zone from PCW 9 to PCW 12, a transitional period from the first to second trimester, but reduced progressively beginning at PCW 14 (FIG. 2, a,b).

In the unique human fetal RL region, the TCP cell population with robust expression of HNRNPH1 and SOX11 was highly enriched in the RL transitional zone and RLVZ region at PCW 12 compared with other stages, whereas low amounts of TCP signature markers were detected in the RLSVZ region (FIG. 2, c-e). HNRNPH1 and SOX11 were also detected in a population of SOX2+ NSCs in the RL transitional zone (FIG. 2, f).

Trajectory analysis predicted that TCPs may give rise to GCPs (ATOH1+) and UBCs (EOMES+, Eomesodermin) (FIG. 1, c). Consistently, a population of HNRNPH1+ cells that colabelled with EOMES and PAX6 in the RL transitional zone was detected (FIG. 2, g,h), wherein PAX6+ progenitors can give rise to both GCPs and UBCs. This suggests a potential lineage trajectory from TCPs to UBCs through PAX6+ intermediates. A population of ATOH1+ GCP progenitors in the posterior external granule cell layer were co-labelled with HNRNPH1. Pseudo-temporal ordering of cell-state evolution by Slingshot and STREAM revealed a trajectory initiated in NSC branches through the TCP subpopulation, which may serve as a precursor to generate GCP, UBC and Purkinje lineage cells (FIG. 2, i).

### TCP-Like Cells in Cerebellar MBs

To identify progenitor cells with molecular features of cerebellar MBs, fetal cerebellar cell profiles were compared to bulk transcriptomes of MB cohorts from the Children Brain Tumor Tissue Consortium using CIBERSORTx. Consistent with previous observations, the transcriptomic signatures of SHH MB from children and infants had strong similarity to GCP and child SHH tissues also showed weak similarity to TCP. The transcriptome profiles of G4 MBs resembled that of UBCs, whereas G3-MB cells (including MYChigh and MYClow tumors) had the strongest similarity to human fetal TCPs, followed by UBC-lineage cells.

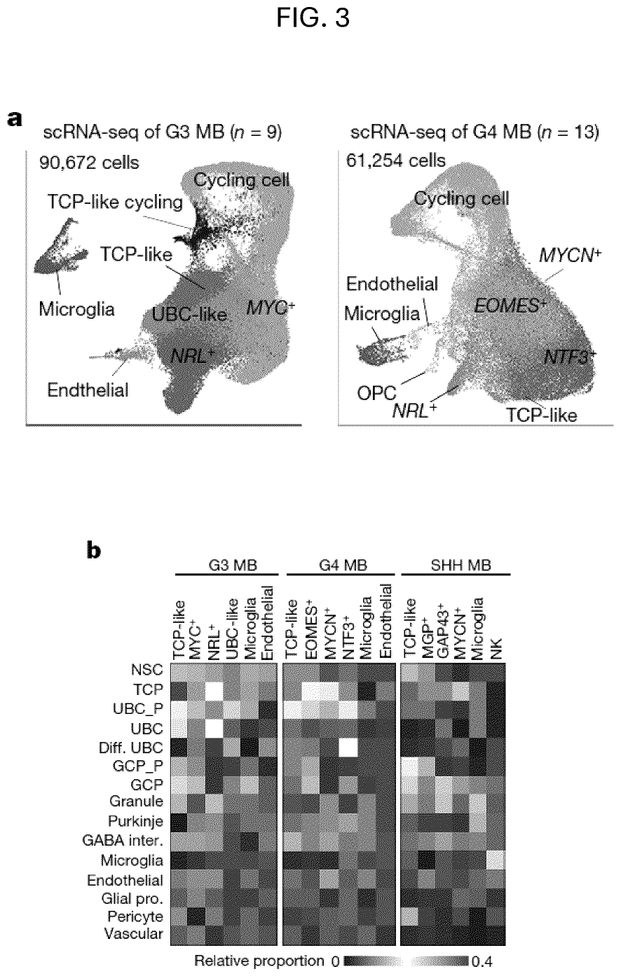

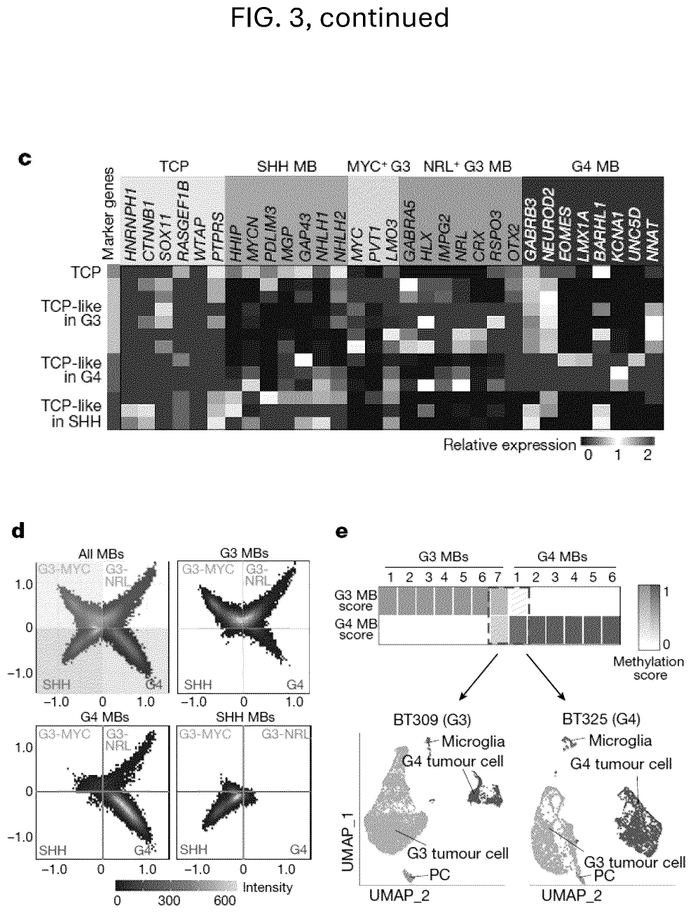

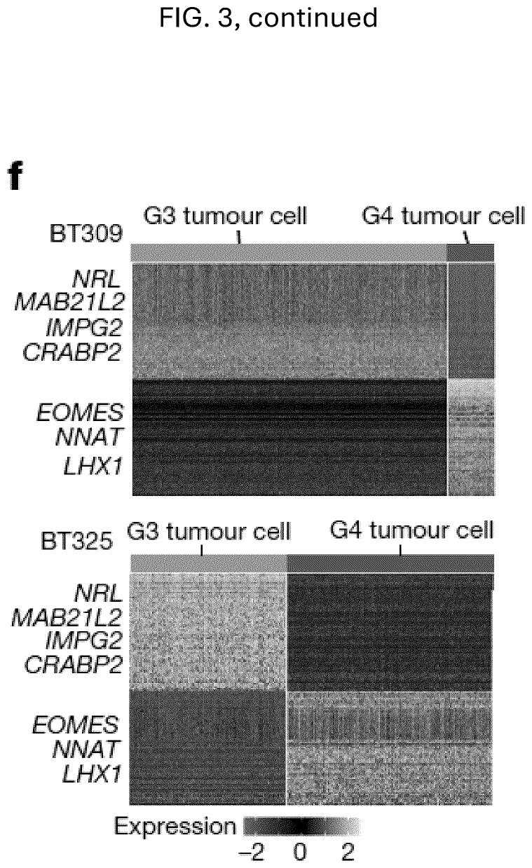

To further define the cellular identity of cerebellar MBs, scRNA-seq and single-nuclei assay were performed for transposase-accessible chromatin with sequencing (snATAC-seq) in matched tissues from 26 MBs. Previously reported transcriptomics data was also included in the analysis. TCP-like populations were identified in G3, G4 and SHH MBs as were tumor-subtype-specific cell clusters (FIG. 3a). Unsupervised VECTOR trajectory analysis predicted that TCP-like cells were in an undifferentiated state. Reciprocal analysis of the overlaps between tumor cells and primary fetal cerebellar tissues using ProjecTILs revealed that MB tumor cells included TCP-like cell populations analogous to those in the human fetal cerebellum. TCP-like cells in different MB subgroups transcriptionally mimicked the normal TCP populations, whereas neoplastic cells in G3 and G4 MBs and in SHH MBs also had gene signatures similar to UBC lineage cells and GCPs, respectively (FIG. 3, b). CIBERSORTx analysis showed that TCP-like populations were present in higher abundances in G3 MBs than in G4 and SHH MBs. TCP-like cells in MB tumors showed the TCP signatures (FIG. 3, c) including expression of HNRNPH1 and SOX11 (elevated in a variety of cancers) and CTNNB1 (drives tumor formation in the cerebellum if normal lineage restriction is lifted). TCP-like populations in G3, G4 and SHH MBs also expressed subgroup-specific signatures (FIG. 3, c). These observations indicate that there is a potential tumorigenic evolution of TCP cells into specific neoplastic TCP-like cells in individual MB subgroups, possibly caused by distinct driver mutations.

Cell-state plots revealed specific enrichment of tumor cell states associated with MB subgroups (FIG. 3, d). In contrast to the relatively restricted tumor cell state in SHH MB, MYC+ and NRL+ cell states were detected as well as a state with a G4 MB-like signature in G3 MBs (FIG. 3, d). The G4 MB tumors were enriched in G4 MB-specific states and a cell state with an NRL+G3 signature (FIG. 3, d). These observations indicate that a cohort of G3 and G4 tumors have cells with either mixed or intermediate plastic states.

### Tumor Cell Populations in G3 and G4 MBs

G3 and G4 MBs share similar signature genes based on bulk transcriptome and DNA methylation profiles. To evaluate the hypothesis that intermediate cell populations are shared between G3 and G4 tumors, two MBs (BT-309 and BT-325) were analyzed that showed a mixture of G3 and G4 signatures based on a DNA methylation profiling (FIG. 3, e). Unbiased single-cell clustering analysis of transcriptomes revealed distinct G3 and G4 tumor cell populations without substantial intermediate states expressing both G3 and G4 signatures (FIG. 3, f), and single-cell copy number variation (CNV) analysis confirmed that G3 and G4-like populations with distinct patterns were present within individual tumors. These data indicate that a set of G3 and G4 MB tumors might harbor a mixture of G3- and G4-specific cell populations rather than a G3/G4 cell-state continuum.

### TCP Frequency Increases During Metastasis

The BT-325 tumor, which harbored both G3 and G4 tumor cells, metastasized to the leptomeningeal surface of the brain. The metastatic tumor had increased frequencies of both TCP-like cells and MYC+G3-like cells but there was a decrease in G4-like cells. There was enrichment in TCP-like and MYC+G3-like states and gene signatures in the metastatic tumor coupled with a decrease in EOMES+G4-like states when compared to the primary tumor. CNV analysis confirmed MYC gene amplification on chromosome 8 in the metastatic tumor in accordance with the higher amount of MYC expression compared to the primary tumor. Similar observations were made in further paired primary and metastatic G3 tumors concordant with the high rate of metastasis in G3 tumors.

Networks that Drive TCP Transformation

To decipher how dynamic accessibility at cis-regulatory elements (CREs) relates to the gene regulatory programs in TCP-like cells from aggressive MBs, snATAC-seq of G3 and G4 MBs were performed for matched scRNA-seq data where available. By correlation of accessibility of promoter and gene body elements with target gene expression using ArchR, the positively correlated peak-to-gene pairs were found to be mostly subcluster-specific.

To determine the temporal relationship between chromatin accessibility and gene expression, the peak-to-gene pairs were ordered based on their CRE accessibility as a function of pseudo-time. In G3 tumors, the TCP-like cluster preceded the MYC+ cell cluster, which was followed by NRL+ cell cluster. Motif analysis indicated an enrichment of binding motifs for SOX11 and TWIST1 in the TCP-like cells, whereas TCF3 and MYC were enriched in the MYC+G3 cells, and NR2F1 and PAX5 motifs were enriched in the NRL+G3 cells. Gene ontology analysis of G3-MB clusters showed an enrichment of epithelial development, epithelial-to-mesenchymal transition (EMT) and TGFb/BMP signalling in the TCP-like population. MYC+ populations, in contrast, were enriched in cell cycle, cell migration and Notch signalling, whereas NRL+ cells were enriched in photoreceptor cell development and Hippo signalling. The enrichment in EMT, TGFβ signalling and cell migration probably contributes to the high metastatic potential of G3 MBs.

In G4 MBs, the accessibility in TCP-like cells emerged before that of the KCNA1+ and EOMES+ cell clusters. An enrichment of epithelial cell development, neural progenitor cells and UBC signatures was observed in TCP-like cells in G4 tumors. The KCNA1+ clusters were enriched in cell-cell adhesion, regulation of neuronal progenitors and MAPK signalling, whereas the EOMES+G4 subpopulation was enriched in neuronal development, HIF-1 and PI3K signalling. In EOMES+G4 subpopulations, FOXG1 and LMX1A motifs were enriched, whereas RORA and PKNOX1/2 motifs were enriched in KCNA1+G4 subpopulations.

To identify positive transcriptional regulators that control gene expression in TCP-like-cell populations, snATAC-seq and scRNA-seq data were integrated to identify transcription factors with gene expression scores positively correlated with changes in accessibility of corresponding motifs. TCP-like cell populations in G3 and G4 MBs were enriched in HLX, CRX, OTX2, BARHL1 and LMX1A in addition to TCP markers HNRNPH1 and SOX11. CRE sites co-accessible with the promoters of potential drivers for MBs, including OTX2 and HLX, were detected in TCP-like cells in G3 tumors, whereas BARHL1 and PAX6 were enriched in TCP-like cells in G4 tumors. Co-accessibility of regulatory CREs and target gene loci might contribute to expression of MB subtype-specific drivers and their oncogenic programs.

### Long-Range Enhancer Hijacking in G3 MB

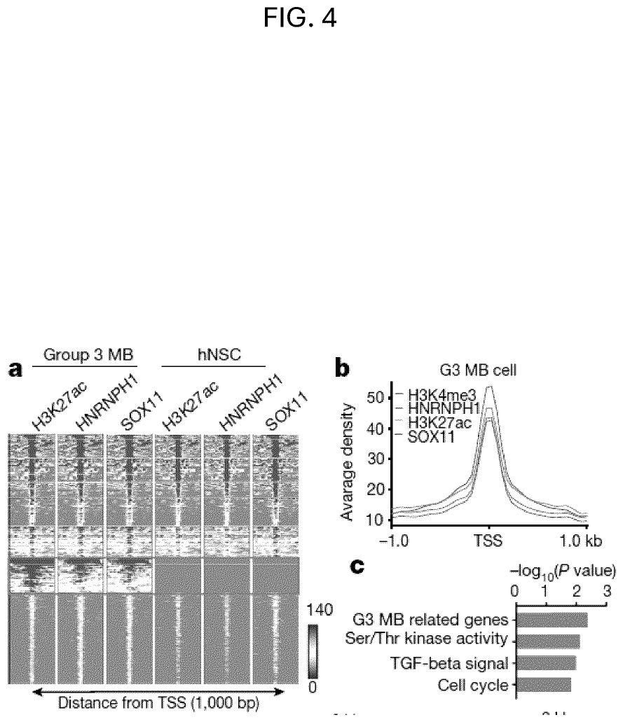

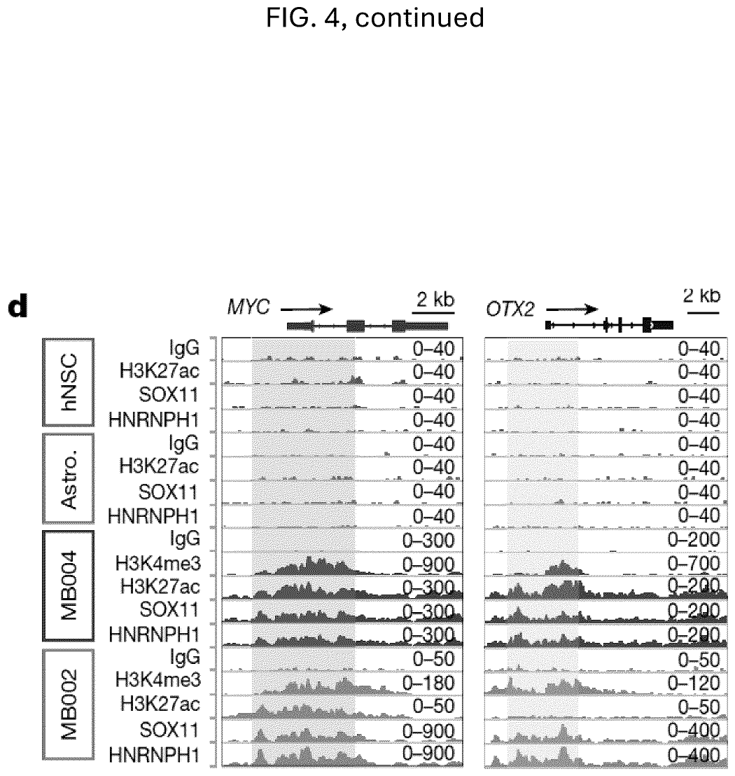

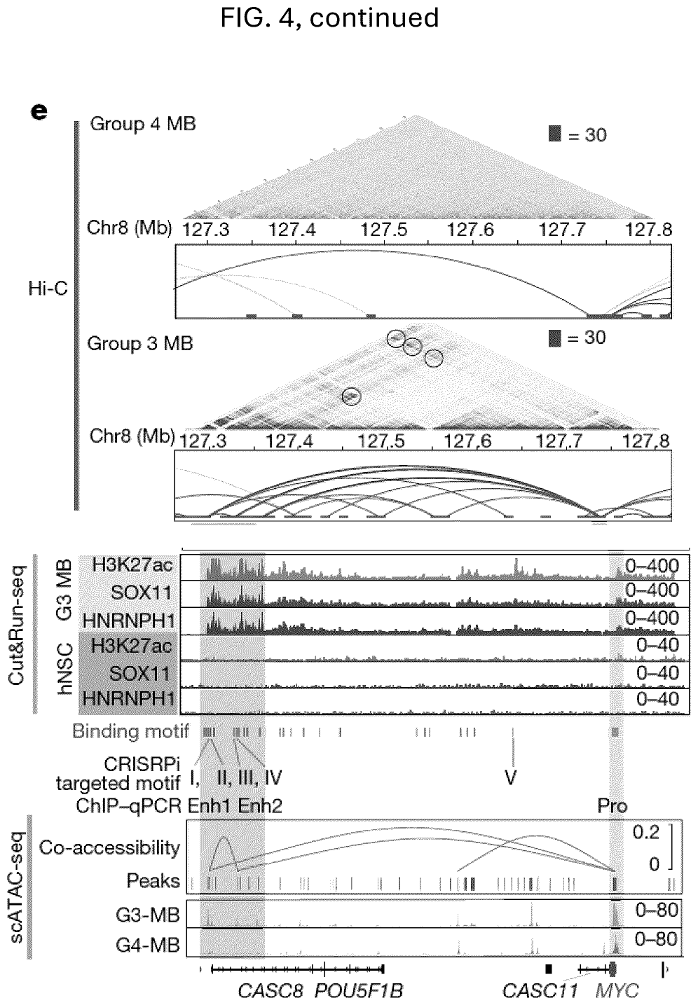

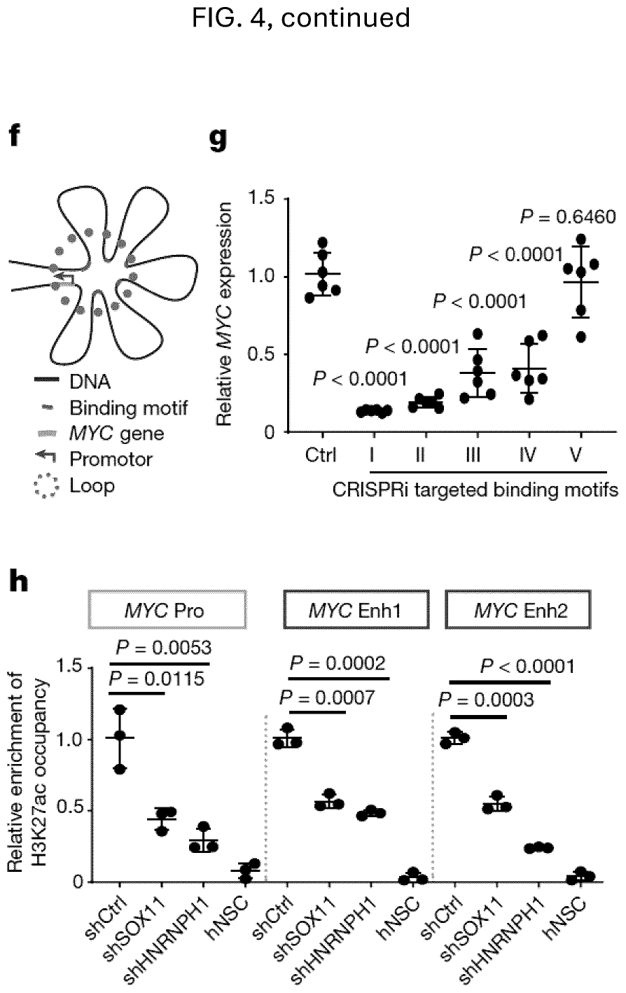

To identify the direct targets of SOX11 and HNRNPH1, a Cut&Run genomic occupancy assay was performed in patient-derived MYC-driven G3 MB tumor cell lines (MB-004 and MB-002) and non-transformed human NSCs and astrocytes (FIG. 4, a). HNRNPH1 and SOX11 co-occupied enhancer and promoter regions near transcriptional start sites (TSSs) marked by activating histone marks H3K27ac and H3K4me3 (FIG. 4, b). H3K27ac signals were higher in G3 MB cells than NSCs or astrocytes. HNRNPH1 and SOX11 targeted common and unique genomic loci in G3 MB cells (FIG. 4, a, boxed). Gene ontology analysis indicated that the unique targets in G3 MB cells were associated with the genes related to G3-MB oncogenesis, Ser/Thr kinase signalling, TGFβ/BMP signalling and cell-cycle regulation (FIG. 4, c). Expression of these genes was higher in G3 MB cells than control cells. Notably, HNRNPH1 and SOX11 targeted G3 signature genes MYC and OTX2 in G3-MB cells but not in control cells (FIG. 4, d). HOMER analysis of consensus sequence motifs associated with sites targeted by HNRNPH1-SOX11 revealed binding motifs for TGFβ/SMAD4, TCF3 and HIPPO/TEAD4, which regulate tumor growth and metastasis. Thus, the results indicate that HNRNPH1 and SOX11 might cooperate with these factors to regulate downstream gene expression that drives the G3 tumorigenic and metastatic programs.

To determine whether SOX11/HNRNPH1-occupied enhancers correspond to distal regulatory elements for activation of G3-MB driver genes, Hi-C chromosome conformation capture was performed in patient-derived G3 MB (MB-004) and G4 MB (UPN3550) cells and detected unique genomic looping in each line. NeoLoopFinder was used to reconstruct local Hi-C maps surrounding breakpoints. Unique structural variations and distinct sets of the interacting genomic loci involved in neo-loop formation at loop anchors in G3 and G4-MB cells were identified. The neo-loop formation through interchromosomal translocation in G3-MB cells placed potential promoter/enhancer elements on a chromosome 11 segment close to the promoter of PPP1R14A on chromosome 19, which has been shown to drive oncogenic RAS signalling in human cancers. Expression of PPP1R14A was higher in the G3 MBs than SHH and G4 MBs. This indicates that interchromosomal translocation or structural variations might activate oncogenic drivers through enhancer hijacking in G3 MBs.

Hi-C analysis indicated that the topologically associated domains in G3-MB cells harbored unique long-distance interactions with the enhancer and promoter regions of MYC (FIG. 4, e). Potential super-enhancers at the regulatory elements near the gene loci such as OTX2, DUX4, CASC8 and MYC in G3-MB cells were identified. By integrating the long-range interacting sites with enhancer occupancy of HNRNPH1/SOX11 in G3-MB cells and NSCs, chromatin interaction loops linking the distal super-enhancer clusters bound by HNRNPH1/SOX11 upstream of CASC8 to the active promoter/enhancer elements of the MYC locus in the G3-MB cells but not in NSCs were detected (FIG. 4, e). This suggests that long-range chromatin looping of super-enhancers juxtaposing to the MYC locus may promote MYC expression in G3-MB cells. CRISPR interference (CRISPRi) targeting of SOX11/HNRNPH1-binding motifs in the distal super-enhancers, but not a control enhancer site, resulting in substantial downregulation of MYC expression (FIG. 4 (f,g)). Sequences used were SEQ ID NO: 1-8. In addition, chromatin immunoprecipitation with quantitative PCR (ChIP-qPCR) showed that the amount of H3K27ac occupancies of enhancers and promoters of MYC or OTX2 in G3-MB cells were reduced substantially when either SOX11 or HNRNPH1 was knocked down (FIG. 4, h. This long-range interaction loop was not detected in G4-MB cells (FIG. 4e), suggesting that a unique enhancer-hijacking event in the MYC locus drives oncogenesis in G3 MB.

### TCP-Like Signature Genes in G3-MB Growth

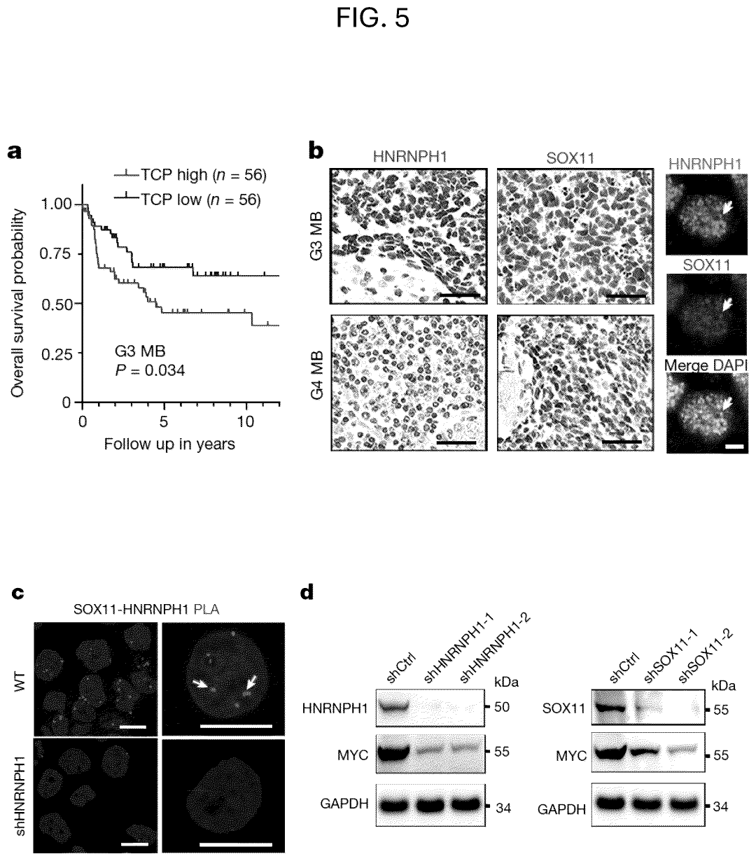

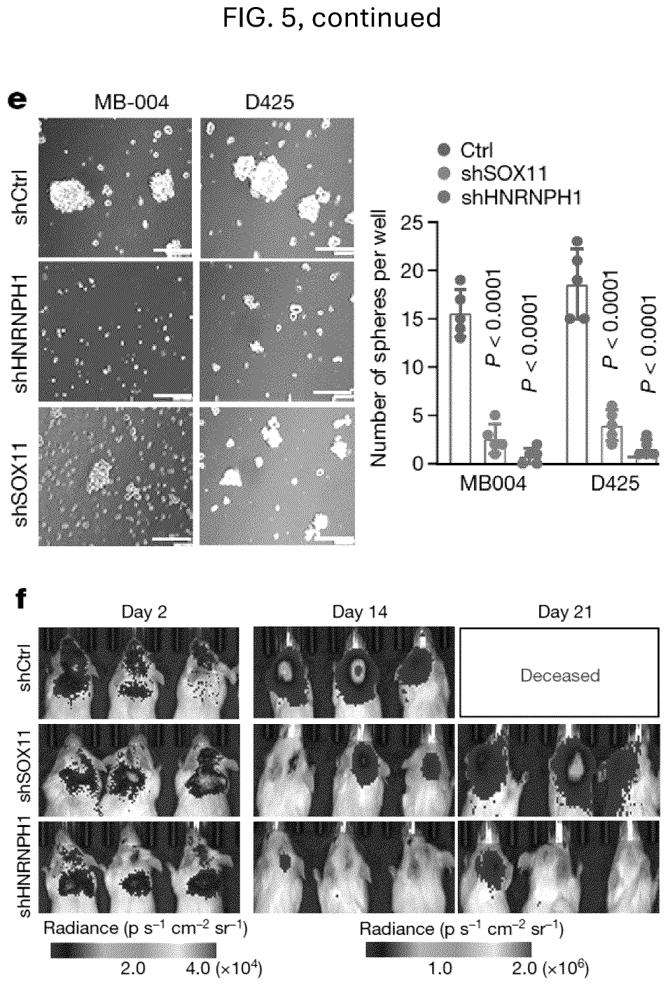

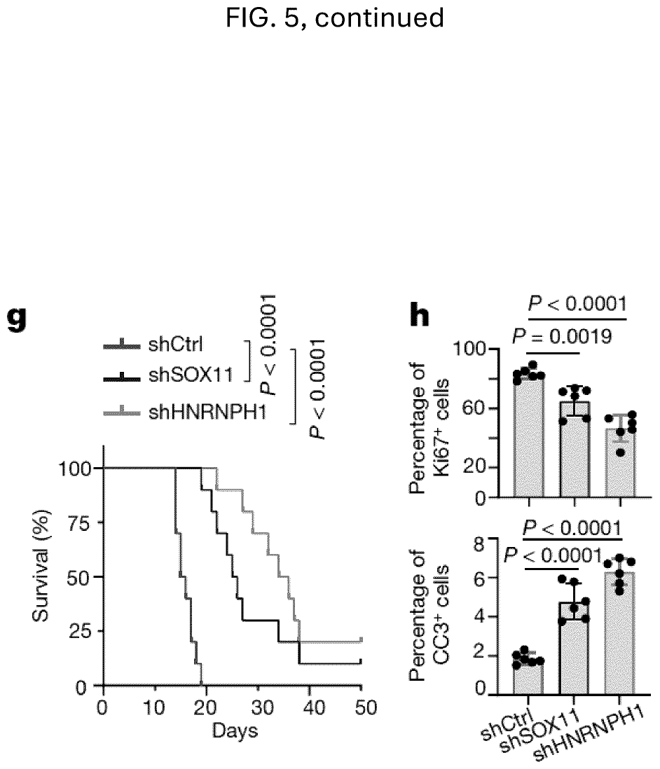

On the basis of the TCGA dataset, the amount of TCP marker HNRNPH1 was higher in G3 MBs than other MB subgroups, whereas SOX11 expression was the highest in G4 MB (data not shown). Notably, patients with high TCP scores (proportion of TCP) in G3 MB had worse prognosis than those with low TCP scores (FIG. 5, a), whereas the prognostic impact was not observed in other MB subgroups. HNRNPH1 and SOX11 were detected in G3 and G4-MB tissues and co-expression of HNRNPH1 and SOX11 in a population of G3-MB cells (FIG. 5, b). Proximity ligation assay showed that expression of HNRNPH1 and SOX11 was detected in the same foci in the nuclei of G3 tumor cells, and depletion of HNRNPH1 abrogated the signal (FIG. 5, c). Consistent with their co-occupancy on CREs, these data indicate that HNRNPH1 may directly interact with SOX11 in G3-MB tumor cells.

To determine the roles of HNRNPH1 and SOX11 in the growth of G3 and G4-MB tumor cells, tumor cells of HNRNPH1 or SOX11 were depleted using short hairpin RNAs. Silencing of HNRNPH1 or SOX11 in patient-derived G3-MB cells substantially reduced expression of MYC (FIG. 5, d) and genes related to the G3-MB-associated signature whereas expression of neuronal differentiation genes increased. In addition, depletion of HNRNPH1 or SOX11 reduced tumor sphere formation in G3-derived MB-004 and D425 cells (FIG. 5e). Moreover, the growth of the patient-derived G3 MB-004 cells and of G4 MB UPN3550 cells was substantially reduced on HNRNPH1 or SOX11 silencing, whereas depletion of either factor did not substantially impair the growth of NSCs or astrocytes. Silencing HNRNPH1 or SOX11 also significantly increased cell apoptosis. Furthermore, an in vivo xenograft analysis showed that SOX11 or HNRNPH1 depletion substantially inhibited the growth of G3-MB tumors and prolonged animal survival (FIG. 5, f, g). The tumors depleted of HNRNPH1 or SOX11 had decreased proportions of Ki67+ proliferative cells and increased apoptosis (FIG. 5, h). These results indicate that expression of the TCP signature genes HNRNPH1 and SOX11 is critical for the growth of aggressive G3-MB tumors.

### Discussion

In this study, a previously unrecognized transitional progenitor population in the human fetal cerebellum was identified. These cells were abundant during a narrow time window around the first-to-second-trimester transition stage, a period critical for neuronal lineage specification, proliferation and migration, and diminished thereafter. These progenitors had stem-like features of undifferentiated and transitory cell states with the potential to give rise to different cerebellar cell types including UBCs, GCPs and Purkinje cell lineages. A recent study using snRNA-seq profiling of frozen fetal cerebella identified human RL cells but not the TCP subpopulation. The TCP gene signature partially overlaps that of RL cells, yet the populations are distinct. Whole-cell scRNA-seq identifies cell types more representative of cell populations in the starting tissues than does snRNA-seq, which might account for study differences.

Although SOX11+/HNRNPH1+ cells are sparsely distributed throughout mouse embryonic cerebella, they are not enriched in the RL region, in stark contrast to the enrichment of TCP cells in the human cerebellar RL transitional zone and RLVZ, the evolutionarily expanded region in humans. This suggests that TCP cells represent cerebellar intermediate precursors similar to transit-amplifying progenitors involved in human neocortical expansion. Such differences across species might explain why mouse model systems do not fully recapitulate human MB. By intersecting cellular states across developing fetal cerebella and MB subgroups, it was discovered that TCP and tumor cell populations might be interconnected by tumor-subtype-specific transitory states. Integrated single-cell omics and lineage trajectory analyses indicate that TCP-like cells might transition towards G3 tumors, serving as a potential cell of origin for G3 MB in a subset of tumors that form in infancy. Given the similarity between fetal and tumor cells does not necessarily indicate the tumor cell of origin, it is possible that malignant transformation occurs in other lineage precursors such as UBC lineage progenitors or through de-differentiation into TCP-like cells. The subgroup-specific transitory TCP-like progenitors within different MB subgroups may reflect intrinsic oncogenic mutations and cellular plasticity of TCP cells along distinct lineage trajectories, which may contribute to inter- and intra-tumoral heterogeneity as well as therapy resistance.

Data from a recent study using SMART-seq or bulk RNA-seq indicated a cell-state continuum among G3 and G4 MBs. However, unbiased single-cell clustering predicted that distinct populations of prototypical G3 and G4 tumor cells are present within G3/G4 tumors as opposed to a range between G3 and G4 tumor cells. The limited gene sets for G3/G4 subtyping might diminish the distinction between cell types; a much higher number of cells and genes were assessed in this study. The data indicate that G3 and G4 MB cell populations might not interconvert in a subset of G3/G4 tumors, although the data do not exclude the possibility of an intermediate G3/G4 state.

Single-cell profiling of paired primary and metastatic MBs revealed a substantial increase in the proportion of transitional TCP cells and MYC+ tumor cells in the metastatic tumors, suggesting that the TCP-like subpopulation and G3 tumor lineage cells drive metastatic tumor formation. A TCP-like cell score was found to be associated with poor prognosis in G3 MB but not in other subgroups. The higher proportions of the TCP-like cells in G3 MBs might contribute to the difference in survival outcomes. A TCP-like cluster was not detected in brainstem-derived WNT MB, which has the best prognosis of MB subgroups. Integrated scRNA-seq and snATAC-seq analyses identified transcriptional regulatory networks in TCP-like populations. Targeting TCP-like cells through depletion of HNRNPH1 or SOX11 inhibited the growth of the aggressive G3-tumor cells.

Moreover, 3D-chromatin structure analysis revealed long-distance spatial looping of HNRNPH1/SOX11-bound super-enhancers juxtaposed to MYC promoter/enhancer elements, which was uniquely present in MYC-driven G3-tumor cells but not G4 tumor or NSC cells. Thus, TCP cell identity determinants HNRNPH1 and SOX11, which are upregulated in several cancers, not only define the TCP-like state but also hijack long-range super-enhancers to promote expression of oncogenes. Together, the data provide insights into the potential origin, lineage plasticity and human-specific nature of MB subtypes, as well as their intra- and intertumoral heterogeneity in malignancy and metastasis, while revealing a targetable vulnerability for therapeutic intervention of aggressive MB.

### REFERENCES

1. Wang, J., Garancher, A., Ramaswamy, V. & Wechsler-Reya, R. J. Medulloblastoma: from molecular subgroups to molecular targeted therapies. Annu. Rev. Neurosci. 41, 207-232 (2018).
2. Cavalli, F. M. G. et al. Intertumoral heterogeneity within medulloblastoma subgroups. Cancer Cell 31, 737-754.e736 (2017).
3. Vladoiu, M. C. et al. Childhood cerebellar tumors mirror conserved fetal transcriptional programs. Nature 572, 67-73 (2019).
4. Hovestadt, V. et al. Resolving medulloblastoma cellular architecture by single-cell genomics. Nature 572, 74-79 (2019).
5. Aldinger, K. A. et al. Spatial and cell type transcriptional landscape of human cerebellar development. Nat. Neurosci. 24, 1163-1175 (2021). Nature|Vol 612|22/29 Dec. 2022|793
6. Gibson, P. et al. Subtypes of medulloblastoma have distinct developmental origins. Nature 468, 1095-1099 (2010).
7. Kool, M. et al. Molecular subgroups of medulloblastoma: an international meta-analysis of transcriptome, genetic aberrations, and clinical data of WNT, SHH, Group 3, and Group 4 medulloblastomas. Acta Neuropathol. 123, 473-484 (2012).
8. Pugh, T. J. et al. Medulloblastoma exome sequencing uncovers subtype-specific somatic mutations. Nature 488, 106-110 (2012).
9. Roussel, M. F. & Hatten, M. E. Cerebellum development and medulloblastoma. Curr. Top Dev. Biol. 94, 235-282 (2011).
10. Haldipur, P. et al. Spatiotemporal expansion of primary progenitor zones in the developing human cerebellum. Science 366, 454-460 (2019).
11. McGinnis, C. S., Murrow, L. M. & Gartner, Z. J. DoubletFinder: doublet detection in singlecell RNA sequencing data using artificial nearest neighbors. Cell Syst. 8, 329-337 e324 (2019).
12. Becht, E. et al. Dimensionality reduction for visualizing single-cell data using UMAP. Nat. Biotechnol. 37, 38-44 (2019).
13. Qiu, X. et al. Reversed graph embedding resolves complex single-cell trajectories. Nat. Methods 14, 979-982 (2017).
14. Chen, H. et al. Single-cell trajectories reconstruction, exploration and mapping of omics data with STREAM. Nat. Commun. 10, 1903 (2019).
15. Street, K. et al. Slingshot: cell lineage and pseudotime inference for single-cell transcriptomics. BMC Genomics 19, 477 (2018).
16. Abdelaal, T. et al. A comparison of automatic cell identification methods for single-cell RNA sequencing data. Genome Biol. 20, 194 (2019).
17. Welch, J. D. et al. Single-cell multi-omic integration compares and contrasts features of brain cell identity. Cell 177, 1873-1887 e1817 (2019).
18. Yeung, J., Ha, T. J., Swanson, D. J. & Goldowitz, D. A novel and multivalent role of Pax6 in cerebellar development. J. Neurosci. 36, 9057-9069 (2016).
19. Newman, A. M. et al. Determining cell type abundance and expression from bulk tissues with digital cytometry. Nat. Biotechnol. 37, 773-782 (2019).
20. Zhang, F., Li, X. & Tian, W. Unsupervised inference of developmental directions for single cells using VECTOR. Cell Rep. 32, 108069 (2020).
21. Andreatta, M. et al. Interpretation of T cell states from single-cell transcriptomics data using reference atlases. Nat. Commun. 12, 2965 (2021).
22. Steen, C. B., Liu, C. L., Alizadeh, A. A. & Newman, A. M. Profiling cell type abundance and expression in bulk tissues with CIBERSORTx. Methods Mol. Biol. 2117, 135-157 (2020).
23. Liu, M. et al. HNRNPH1 is a novel regulator of cellular proliferation and disease progression in chronic myeloid leukemia. Front. Oncol. 11, 682859 (2021).
24. Oliemuller, E. et al. SOX11 promotes epithelial/mesenchymal hybrid state and alters tropism of invasive breast cancer cells. eLife 9, e58374 (2020).
25. Patmore, D. M. et al. DDX3X suppresses the susceptibility of hindbrain lineages to medulloblastoma. Dev. Cell 54, 455-470 e455 (2020).
26. Neftel, C. et al. An integrative model of cellular states, plasticity, and genetics for glioblastoma. Cell 178, 835-849 e821 (2019).
27. Northcott, P. A. et al. The whole-genome landscape of medulloblastoma subtypes. Nature 547, 311-317 (2017).
28. Zapotocky, M. et al. Differential patterns of metastatic dissemination across medulloblastoma subgroups. J. Neurosurg. Pediatr. 21, 145-152 (2018).
29. Granja, J. M. et al. ArchR is a scalable software package for integrative single-cell chromatin accessibility analysis. Nat. Genet. 53, 403-411 (2021).
30. Massague, J. & Ganesh, K. Metastasis-initiating cells and ecosystems. Cancer Discov. 11, 971-994 (2021).
31. Meers, M. P., Bryson, T. D., Henikoff, J. G. & Henikoff, S. Improved CUT&RUN chromatin profiling tools. eLife 8, e46314 (2019).
32. Creyghton, M. P. et al. Histone H3K27ac separates active from poised enhancers and predicts developmental state. Proc. Natl Acad. Sci. USA 107, 21931-21936 (2010).
33. Lamar, J. M. et al. The Hippo pathway target, YAP, promotes metastasis through its TEADinteraction domain. Proc. Natl Acad. Sci. USA 109, E2441-E2450 (2012).
34. Wang, X. et al. Genome-wide detection of enhancer-hijacking events from chromatin interaction data in rearranged genomes. Nat. Methods 18, 661-668 (2021).
35. Riecken, L. B. et al. CPI-17 drives oncogenic Ras signaling in human melanomas via Ezrin-Radixin-Moesin family proteins. Oncotarget 7, 78242-78254 (2016).
36. Hnisz, D. et al. Convergence of developmental and oncogenic signaling pathways at transcriptional super-enhancers. Mol. Cell 58, 362-370 (2015).
37. Volpe, J. J. Cerebellum of the premature infant: rapidly developing, vulnerable, clinically important. J. Child Neurol. 24, 1085-1104 (2009).
38. de Graaf-Peters, V. B. & Hadders-Algra, M. Ontogeny of the human central nervous system: what is happening when? Early Hum. Dev. 82, 257-266 (2006).
39. Slyper, M. et al. A single-cell and single-nucleus RNA-Seq toolbox for fresh and frozen human tumors. Nat. Med. 26, 792-802 (2020).
40. Fischer, J. & Ayers, T. Single nucleus RNA-sequencing: how it's done, applications and limitations. Emerg. Top Life Sci. 5, 687-690 (2021).
41. Lui, J. H., Hansen, D. V. & Kriegstein, A. R. Development and evolution of the human neocortex. Cell 146, 18-36 (2011).
42. Hendrikse, L. D. et al. Failure of human rhombic lip differentiation underlies medulloblastoma formation. Nature 609, 1021-1028 (2022).
43. Smith, K. S. et al. Unified rhombic lip origins of group 3 and group 4 medulloblastoma. Nature 609, 1012-1020 (2022).
44. Yuan, S., Norgard, R. J. & Stanger, B. Z. Cellular plasticity in cancer. Cancer Discov. 9, 837-851 (2019).
45. Williamson, D. et al. Medulloblastoma group 3 and 4 tumors comprise a clinically and biologically significant expression continuum reflecting human cerebellar development. Cell Rep. 40, 111162 (2022).
46. Riemondy, K. A. et al. Neoplastic and immune single-cell transcriptomics define subgroup specific intra-tumoral heterogeneity of childhood medulloblastoma. Neuro. Oncol. 24, 273-286 (2022).

### Methods

### Human Samples and Tumor Tissue Collection

Human fetal and tumor tissues were obtained from the Children's Hospital of Fudan University and XinHua Hospital at the Shanghai Jiao Tong University School of Medicine, and Obstetrics and Gynecology Hospital of Fudan University. Informed consents for the use of tissues for research were obtained in writing from donors or the patients' parents in this study. The fetal and tumor tissue collections were approved by the individual institutional review board at the Children's Hospital of Fudan University, XinHua Hospital at the Shanghai Jiao Tong University School of Medicine, and Obstetrics and Gynecology Hospital of Fudan University. Tumor tissue collections were approved by institutional review board at the Cincinnati Children's Hospital Medical Center (CCHMC). Fresh cerebellar tissues from aborted fetuses and tumors after surgery were collected and digested by collagenase IV (2 μg ml−1, Thermo Fisher, catalogue no. 17104019) enzymatic dissociation for 20 min at 37° C. after mechanically dissociation followed by single-cell profiling.

### Animal Experiments

Immunodeficient NOD SCID gamma (NSG) 8-14-week-old mice were obtained from the CCHMC animal core. Mice of either sex were used and fed (four mice or fewer per cage) in the vivarium. All studies complied with all relevant animal use guidelines and ethical regulations. The animal studies were approved by the IACUC (Institutional Animal Care and Use Committees) of the CCHMC. In the xenograft model, MB tumor cells were transduced with lentivirus targeting HNRNPH1 or SOX11 for 20 h and 2×10⁵ cells suspended in 3 μl of PBS with 1 μl of Matrigel (Corning, no. 356234) were stereotactically injected into the cerebellum of NSG mice. Animals were monitored and bioluminescence images were captured weekly. Animals were euthanized when they reached the tumor endpoints defined by body weight loss >20% or poor health condition according to the IACUC protocol. These limits were not exceeded in any of these experiments. Mice were housed at room temperature (20-23° C.) with a 12-h light-dark cycle set with lights on from 06:00 to 18:00 and with humidity between 30 and 80%. All the animal experiments were performed in accordance with the guidelines established by IACUC at the CCHMC. Animal survival endpoint is the date of the animal that died or was euthanized according to animal use guidelines. The limits of endpoints were not exceeded in any of the experiments.

### MB Cell Line Culture

MB cell lines D425 (catalogue no. SCC290, Millipore sigma), DAOY (HTB-186, ATCC), D458 (CVCL_1161, Cellosaurus) and D283 (HTB-185, ATCC) were cultured in the DMEM/F12 (Thermo Fisher, no. 11320033) with 10% FBS and 2 mM 1-glutamine and 1% penicillin/streptomycin. UPN3550 cells were isolated from tissue taken from a patient with a G4 MB primary tumor and cultured in DMEM/F12 with 10% FBS and 2 mM1-glutamine and 1% penicillin/streptomycin, which was proved by the institutional review board at the Cincinnati Children's Hospital. MB004 and MB002 G3 MB lines were provided by M. Roussel and cultured in neurobasal medium (Sigma, SCM003) with 2% B-27, 1 μg ml-1 heparin, 2 mM 1-glutamine and 1% penicillin/streptomycin, 25 ng ml-1 fibroblast growth factor (FGF) and 25 ng ml-1 epidermal growth factor (EGF) at 37° C. in an atmosphere of 5% CO2.

Immunostaining, immunohistochemistry and immunoblotting The immunostaining procedures followed the method as previously described. Briefly, cerebellar tissues were fixed with 4% PFA for 45 min, washed five times with PBS and dehydrated with 30% sucrose overnight, then blocked with optimal cutting temperature frozen embedding media (CRYO-4, Polarstat Inc.) and cryosectioned at 14 μm thickness.

For adherent cells, cells were seeded on the coverslips and fixed with 4% PFA for 10 min and washed five times with PBS, then in blocking solution for 30 min. Primary antibodies were used, including mouse anti-SOX2 (Santa Cruz Biotechnology; catalogue no. sc-365964), rabbit anti-SOX11 (Sigma, catalogue no. HPA000536; Thermo Fisher, catalogue no. 14-9773-82), rabbit anti-HNRNPH1 (Abcam, catalogue no. ab154894; Bethyl Laboratories, catalogue no. A300-511A), rat anti-EOMES (Invitrogen, catalogue no. 14-4875-52), mouse anti-PAX6 (Santa Cruz Biotechnology, catalogue no. sc-81649), mouse anti-ATOH1 (Thermo Fisher, catalogue no. H00000474-M09), rabbit anti-c-Myc (Cell Signaling, catalogue no. 5605), rabbit anti-Ki67 (Thermo Fisher, catalogue no. MA5-14520), rabbit anti-Cleaved Caspase 3 (Cell Signaling, catalogue no. 9661) and mouse anti-BrdU (BD Bioscience, 1:500) antibody with proper dilutions. For BrdU staining, BrdU pulse-labelled (10 μM, 2 h at 37° C.) cells were denatured with 0.1 M HCl for 1 h in a water bath at 37° C. After denaturation, cells were neutralized with 0.1 M Borax, pH 8.5 (Sigma) for 10 min. Cells were washed with PBS three times and blocked with 5% normal donkey serum (Sigma-Aldrich) in wash buffer for 1 h at room temperature. Secondary antibodies conjugated to Cy2, Cy3 or Cy5 were from Jackson ImmunoResearch Laboratories. Tissues or cells were mounted with Fluoromount-G (SouthernBiotech) for microscopy. Immunofluorescence-labelled images were acquired using a Nikon C2+ confocal microscope. Cell images were quantified in a blinded manner.

For paraffin-embedded tissues, sections were dewaxed and hydrated using xylene and ethanol, respectively. Antigen retrieval was performed before permeabilization as previously described. Slides were treated in 0.6% H₂O₂ in methanol for 30 min at 37° C. and blocked in 5% normal donkey serum in PBS with Tween for 1 h at room temperature. SOX11 and HNRNPH1-expressing cells in MB tissues were quantified using the described methods49. In brief, 0-5 denote different degrees (intensity and density) of IHC staining; 5 is the maximum and 0 is the minimum degree. The final score of the patients was equal to SI (score of intensity)×SD (score of density).

For the western blot analysis, cells were lysed with radioimmunoprecipitation assay lysis buffer (Millipore) supplemented with phosphatase and protease inhibitor cocktail (Roche). Protein concentration of each sample was determined by BCA assay using the BCA kit (Beyotime) according to manufacturer's instructions and equal amounts (5-15 μl) were loaded and separated by 12% SDS-PAGE gel. Polyvinyl difluoride membrane (Millipore) was used for gel transfer and the membrane was probed with primary antibodies as indicated, followed by secondary antibodies conjugated with horseradish peroxidase. The signal was detected with Super Signal West Pico/Femto Chemiluminescent Substrate (Thermo Scientific).

### Generation and Processing of DNA Methylation Data

All of the patients' single-cell MB samples sequenced in this study were analysed using Illumina Infinium Methylation EPIC BeadChip arrays according to the manufacturer's instructions. Data were generated from total genome DNA isolated from freshly frozen tissue samples. MB subgroup predictions were obtained from a web-platform for DNA methylation-based classification of central nervous system tumors (https://www.molecularneuropathology.org/mnp). Resulting assignment of samples to SHH, G3 and G4 subgroups were used for all downstream analyses. CNV analysis from EPIC methylation array data was performed using the conumee Bioconductor package (http://bioconductor.org/packages/conumee/).

scRNA-Seq and scATAC-Seq Using 10× Genomics Platform

For scRNA-seq on the 10× Genomics platform, single cells were processed through the GemCode Single Cell Platform using the GemCode Gel Bead, ChIP and Library Kits (10× Genomics) according to the manufacturer's instructions. The concentration of the single-cell suspension was assessed with a Trypan blue count and the sample was used if there were more than 90% viable cells. Roughly 10,000-30,000 cells per sample were loaded on the Chromium Controller and generated single-cell GEM (gel beads in emulsion). GEM-reverse-transcription, DynaBeads clean-up, PCR amplification and SPRIselect beads clean-up were performed using Chromium Single Cell 3′ Gel Bead kit. Indexed single-cell libraries were generated using the Chromium Single Cell 3′ Library kit and the Chromium i7 Multiplex kit. Size, quality, concentration and purity of the complementary DNAs and the corresponding 10× library was evaluated by the Agilent 2100 Bioanalyzer system. Amplified cDNA and final libraries were assessed on an Agilent BioAnalyzer using a High Sensitivity DNA Kit (Agilent Technologies).

For snATAC-seq on the 10× Genomics platform, single-cell libraries were generated using the GemCode Single-cell instruments and the Single Cell ATAC Library & Gel Bead Kit and ChIP Kit from 10× Genomics, according to the manufacturer's instructions. The samples were incubated at 37° C. for 1 h with 10 μl of transposition mix (per reaction, 7 μl of ATAC Buffer and 3 μl of ATAC Enzyme (10× Genomics)). Following the generation of nanoliter-scale GEMs, GEMs were reverse transcribed in a C1000 Touch Thermal Cycler (Bio-Rad) programmed at 72° C. for 5 min, 98° C. for 30 s, 12 cycles of 98° C. for 10 s, 59° C. for 30 s, and 72° C. for 1 min and held at 15° C. After reverse transcription, single-cell droplets were broken and the single-strand cDNA was isolated, cleaned up and amplified. Amplified cDNA and final libraries were assessed on an Agilent BioAnalyzer using a High Sensitivity DNA Kit (Agilent Technologies). All the libraries were sequenced on NovaSeq 6000 (Illumina) at a depth of roughly 400 million reads per sample.

scRNA-Seq Processing and Quality Filtering

For 10× genomics datasets, Cellranger v.5.0.1 was used to align reads to the human reference sequence. The raw base call (BCL) files were demultiplexed into FASTQ files. The FASTQ files were aligned to the reference human genome GRCh38 (hg38) to generate raw gene-barcode count matrices. When clustering several samples together, the many runs were aggregated together to normalize on sequencing depth and recomputed the gene-barcode matrices.

For quality control and normalization of scRNA-seq, the Seurat program was used (https://satijalab.org/seurat/articles/pbmc3k_tutorial.html) in R v.4.0.3 by reading in the data from the reads in the output of the Cellranger pipeline prom 10×, returning a unique molecular identified count matrix. Low-quality cells were identified and removed from the datasets based on the cell with <200 genes expressed and high mitochondrial gene content (5 s.d. above the median). Doublets were detected and filtered using the R package DoubletFinder v.2.0.2 with default settings. The cells with low-abundance genes or genes expressed in fewer than three cells were also removed from the datasets. By defaulting in Seurat, a global-scaling normalization method ‘LogNormalize’ that normalized the feature expression measurements for each cell by the total expression, multiplied this by a scale factor (10,000 by default) and log-transformed the result was used. Next, a linear transformation (‘scaling’) was applied, that is a standard preprocessing step using all genes or variable genes. Then, a principal component analysis (PCA) was performed to get the linear dimensional reduction after the data scaling.

### Clustering Analysis, Visualization and Annotation

Clustering analysis was performed with the R package Seurat (v.4.0.3). Highly variable genes were detected using Seurat's pipeline, calculating average expression and dispersion for each gene, diving genes into bins and computing a z-score for dispersion within each bin. A z-score of 0.5 was used as the cut-off of dispersion, with a bottom cut-off of 0.0125 and a high cut-off of 3.0 for average expression. Linear dimensionality reduction was performed using PCA, and statistically significant principal components were selected using the elbow and jackstraw methods from Seurat. The clusters of cells were identified by a shared nearest neighbour (SNN)-modularity-optimization based clustering algorithm from Seurat. These clusters were then visualized using t-SNE, UMAP or Monocle 3. Cluster cell identity was assigned by manual annotation using known cell-type marker genes and computed differentially expressed genes (DEGs) using the FindAllMarkers function in the Seurat package (one-tailed Wilcoxon rank sum test, P values adjusted for multiple testing using the Bonferroni correction. For selecting DEGs, all genes were probed provided they were expressed in at least 25% of cells in either of the two populations compared and the expression difference on a natural log scale was at least 0.2. Manual annotation was performed iteratively, which included validating proposed cell clusters with known markers and further investigating clusters for which the gene signatures indicated more diversity.

### Pseudo-Time Cell-State Trajectory Analysis

For the fetal cerebellum trajectory analysis, cells were grouped using the ‘UMAP’ clustering algorithm. Cell-state transition directions were inferred by Monocle 3, STREAM or VECTOR programs that provide an unsupervised solution for determining the starting cells. For order_cells function in Monocle 3, the barcodes of selected clusters were normalized using Monocle dPFeature or Seurat to remove genes with low expression and perform PCA analysis on the remaining genes, for significant principal components selections. Differential gene expression analysis was performed using a generalized linear model, and the top 1,000 genes per cluster were selected and fitted into a principal graph within each partition using the learn graph function. For Slingshot cellular trajectory analysis of fetal cerebella, the input matrix was filtered and normalized by the R package Seurat and cell types were annotated and provided as labels for Slingshot. For the single-cell pseudo-time trajectory in tumor tissues, cells from the many patients were aggregated to normalize on sequencing depth and recomputed the gene-barcode matrices using canonical correlation analysis.

Deconvolution and overall patient survival analysis CIBERSORTx19 was applied to perform the deconvolution analysis of the bulk and scRNA-seq tumor data against the human cerebellar clusters except mitotic cells. The transcriptomes of the tumor data (bulk RNA-seq or clusters of scRNA-seq) were as the input used mixture online (https://cibersortx.stanford.edu/runcibersortx.php), and the signature matrix input was the human fetal cerebellum cluster expression matrix after removal of cell-cycle-related genes (roughly 1,400), ribosome biogenesis genes (roughly 300), mitochondrial and apoptosis-related genes (roughly 100), to avoid bias in the deconvolution process. Quantile normalization was disabled and 100-500 permutations for significance analysis were run.

Overall survival of the patients with MB was right censored at 12 years and analyzed by the Kaplan-Meier method. Patient cohorts were subgrouped based on the TCP score (the estimated proportion of TCP). The TCP score was calculated based on the previously described scoring system with CIBERSORTx deconvolution analyses of the proportion of TCP cells against bulk transcriptomes of human MB subgroups (DEGs) from Cavalli's MB cohort dataset. P values of survival curves were reported using the log-rank test.

Cell-Cycle Analysis of Human scRNA-Seq Tumor Samples

Cell-cycle phase-specific annotations were used to define the cell-cycle status for each individual cell. Each cell was assigned a score using CellCycleScoring function in R v.4.0.5, on the basis of its expression of G2/M and S phase markers. These marker sets should be anticorrelated in their expression levels, and cells expressing neither are probably not cycling and in G1 phase.

Inferred CNV Analysis from scRNA-Seq

Malignant cells were identified by inferring large-scale chromosomal CNVs in each single cell on the basis of a moving averaged expression profiles across chromosomal intervals by inferCNV. CNV classification was combined with transcriptomic-based clustering and expression of non-malignant marker genes to identify malignant and non-malignant cells. Non-malignant cells showed high expression of specific marker genes and no apparent CNVs.

Filtering Cells by TSS Enrichment and Unique Fragments of the scATAC-Seq

Enrichment of ATAC-seq accessibility at TSSs was used to quantify data quality without the need for a defined peak set. Calculating enrichment at TSSs was performed as previously described, and TSS positions were acquired from the Bioconductor package from TxDb. Hsapiens.UCSC.hg38.knownGene. Briefly, Tn5-corrected insertions were aggregated ±2,000 base pairs (bp) relative to each unique TSS genome-wide (TSS strand-corrected). The calculated TSS enrichment represents the maximum of the smoothed profile at the TSS. All scATAC-seq profiles were filtered to keep those that had at least 1,000 unique fragments and a TSS enrichment of 0.5. To minimize the contribution of potential doublets to the analysis, snATAC-seq profiles that had more than 100,000 unique nuclear fragments were removed.

Gene Regulatory Network and Motif Enrichment Analysis of scRNA-Seq and scATAC-Seq Data

To characterize underlying gene regulatory network and infer transcription factor activities in the scRNA-seq dataset, the single-cell regulatory network inference and clustering package was used to identify gene regulatory modules and retain those with a cis-regulatory binding motif for upstream regulators. scRNA-seq and scATAC-seq datasets were merged to create a common peak set, and quantify this peak set in each experiment. The peak coordinates for each experiment were loaded and converted to genomic ranges using the GenomicRanges::reduce function to create a common set of peaks to quantify in each dataset. The detailed settings and parameters as default according to Signac (https://satijalab.org/signac/) were used. ArchR package was used for integrated scRNA-seq and scATAC-seq analyses according to default parameters, including quality control and cell filtering, dimension reduction, genome browser visualization, gene expression data preprocessing and cell annotation, DNA accessibility data processing, joint data visualization, differential accessibility and motif enrichment.

For nominating the marker genes and potential drivers, ArchR29 was used to identify the enriched transcription factors whose inferred gene scores are correlated to their chrom VAR transcription factor deviation z-scores. The gene scores were calculated on the basis of the summed chromatin accessibility and normalized across all genes to a user-defined constant (default of 10,000) according to the ArchR package. On the basis of the gene scores and positive transcription factor-regulators identified from ArchR, the top 30 transcription factors or highly expressed genes (excluding non-coding genes or ribosomal proteins) were nominated as potential drivers or marker genes.

### Two-Dimensional Representation of SC-Derived Cell States and G3 or 4 B/C Score

Tumor cell clusters were used for computing subtype expression scores for each tumor cell in the datasets as previously described. Cell clusters were separated into G3-MYC, G3-NRL, G4 and SHH-clusters. To visualize the enrichment of subsets of cells, across the two-dimensional representation, the fraction of cells that belong to the respective subset among its 100 nearest neighbors was calculated for each cell, as defined by Euclidean distance, and these fractions were shown in different colors. In addition, the previously defined G3 or 4 B/C score system was used on the basis of selected G3 and G4-expressing genes (top 30 genes from each metaprogram) for the overlapping cell-state analysis in G3 and G4 MBs.

### Generation of Hi-C Libraries and Analysis

MB004 (G3 MB) and UPN3550 (G4 MB) cells were processed for Hi-C library construction using the Arima Hi-C Kit following the manual (Arima Genomics, no. A510008). Briefly, five million cells were cross-linked with 1% formaldehyde for 10 min at room temperature and then quenched with 0.2 M glycine. Cell pellets were washed with cold PBS and lysed with lysis buffer to release nuclei and then permeabilized and in situ digested. KAPA Hyper Prep kit was used for library amplification (KAPA, KK2802). Hi-C libraries were sequenced 2×150 bp on a NovaSeq 6000 instrument (Illumina). Juicer were used to process raw reads and generate Hi-C contact matrices (.hic files), aligning to reference genome hg38 to generate Hi-C contact matrices (.hic files). Contact matrices were visualized using Juicebox.

Bam files were used as input, with low-quality reads filtered out. Peakachu and diffPeakachu was used to call and compare loops in Hi-C data from both G3 MB and G4 MB cell lines in Hi-C data, then used diffPeakachu to compare one cell line with another cell line. Tumor-subtype-specific loops were then merged using BEDTools pair to pair function with a negative slope of 10 kilobases. Hi-C breakfinder pipeline 57 was used to identify large structural translocations, deletions and inversions. To identify neo-loops or enhancer-hijacking events, NeoLoopFinder pipeline was used.

### Targeting Distal Enhancers Using CRISPRi and ChIP-qPCR

MB-004 cells were transduced with the enti-dCas9-KRAB-T2A-GFP virus (Addgene no. 71237). Single-guide RNAs (sgRNAs) targeting SOX11/HNRNPH1-binding motifs in the distal enhancers were designed using the CHOPCHOP program (https://chopchop.cbu.uib.no). Green fluorescent protein (GFP-_reporter positive cells were flow-sorted after 2 days of transduction. DNA oligonucleotides were annealed and ligated into the lentiGuide-Cherry vector (Addgene no. 170510) at the BsmBI restriction enzyme cutting sites. The sgRNA sequences were confirmed by Sanger sequencing. The lentivectors expressing each pair of genomic RNAs targeting distal enhancers were packaged in 293T cells using pMD2.G (Addgene no. 12259) and psPAX (Addgene no. 12260). The lentiGuide-Cherry lentiviruses carrying sgRNAs were concentrated from the virus-containing medium by ultracentrifuging and transduced into dCas9-KRAB-T2A-GFP-expressing MB-004 cells (multiplicity of infection <1). RNAs were then extracted from the GFP+/Cherry+ cells after 72 h culture, and cDNAs were prepared using SuperScript III First-Strand Synthesis System (Invitrogen) according to the manufacturer's instructions. qPCR with reverse transcription (RT-qPCR) was performed to quantify gene expression using SYBR FAST qPCR Master Mix. All sgRNA and RT-qPCR sequences used for validation are provided in Table 1.

TABLE 1

| | | SEQ ID |
|---|---|---|
| Name | SEQUENCE | NO |
| shSOX11-1F | GATCCGCCTCTACTACAGCTTCAAG | SEQ ID |
| (siRNA- | ATTCAAGAGATCTTGAAGCTGTAGT | NO: 1 |
| artificial) | AGAGGCTTTTTG | |
| shSOX11-R | AATTCAAAAAGCCTCTACTACAGCT | SEQ ID |
| (siRNA) | TCAAGATCTCTTGAATCTTGAAGCT | NO: 2 |
| | GTAGTAGAGGCG | |
| shSOX11-2F | GATCCGCAGGAAGATCATGGAGCAG | SEQ ID |
| (siRNA) | TTTCAAGAGAACTGCTCCATGATCT | NO: 3 |
| | TCCTGCTTTTTG | |
| shSOX11-2R | AATTCAAAAAGCAGGAAGATCATGG | SEQ ID |
| (siRNA) | AGCAGTTCTCTTGAAACTGCTCCAT | NO: 4 |
| | GATCTTCCTGCG | |
| shHNRNPH1- | GATCCGGGACACAGATATGTTGAAG | SEQ ID |
| 1F | TTTCAAGAGAACTTCAACATATCTG | NO: 5 |
| (siRNA) | TGTCCCTTTTTG | |
| shHNRNPH1- | AATTCAAAAAGGGACACAGATATGT | SEQ ID |
| 1R | TGAAGTTCTCTTGAAACTTCAACAT | NO: 6 |
| (siRNA) | ATCTGTGTCCCG | |
| shHNRNPH1- | GATCCGGGCTCAAGGTATTCGTTTC | SEQ ID |
| 2F | ATTCAAGAGATGAAACGAATACCTT | NO: 7 |
| (siRNA) | GAGCCCTTTTTG | |
| shHNRNPH1- | AATTCAAAAAGGGCTCAAGGTATTC | SEQ ID |
| 2R | GTTTCATCTCTTGAATGAAACGAAT | NO: 8 |
| (siRNA) | ACCTTGAGCCCG | |
| CRISPRi | CACCGTAAATCAAGCAGATGGTCAC | SEQ ID |
| MYC | | NO: 9 |
| Enhancer | | |
| I F | | |
| (homo | | |
| sapiens) | | |
| CRISPRi | AAACGTGACCATCTGCTTGATTTAC | SEQ ID |
| MYC | | NO: 10 |
| Enhancer | | |
| I R | | |
| (homo | | |
| sapiens) | | |
| CRISPRi | CACCGGGAAGGATGCGGACAGCCAG | SEQ ID |
| MYC | | NO: 11 |
| Enhancer | | |
| II F | | |
| (homo | | |
| sapiens) | | |
| CRISPRi | AAACCTGGCTGTCCGCATCCTTCCC | SEQ ID |
| MYC | | NO: 12 |
| Enhancer | | |
| II R | | |
| (homo | | |
| sapiens) | | |
| CRISPRi | CACCGGACTATGTGACCATATGCCA | SEQ ID |
| MYC | | NO: 13 |
| Enhancer | | |
| III F | | |
| (homo | | |
| sapiens) | | |
| CRISPRi | AAACTGGCATATGGTCACATAGTCC | SEQ ID |
| MYC | | NO: 14 |
| Enhancer | | |
| III R | | |
| (homo | | |
| sapiens) | | |
| CRISPRi | CACCGCAGGCAACATGGCTTGCTAA | SEQ ID |
| MYC | | NO: 15 |
| Enhancer | | |
| IV F | | |
| (homo | | |
| sapiens) | | |
| CRISPRi | AAACTTAGCAAGCCATGTTGCCTGC | SEQ ID |
| MYC | | NO: 16 |
| Enhancer | | |
| IV R | | |
| (homo | | |
| sapiens) | | |
| CRISPRi | CACCGCTTAGCAGCTCCATGATCTG | SEQ ID |
| MYC | | NO: 17 |
| Enhancer | | |
| V F | | |
| (homo | | |
| sapiens) | | |
| CRISPRi | AAACCAGATCATGGAGCTGCTAAGC | SEQ ID |
| MYC | | NO: 18 |
| Enhancer | | |
| V R | | |
| (homo | | |
| sapiens) | | |
| CRISPRi | CACCGGCTACTTGGGAGACTGAGAC | SEQ ID |
| MYC | | NO: 19 |
| Enhancer | | |
| VI F | | |
| (homo | | |
| sapiens) | | |
| CRISPRi | AAACGTCTCAGTCTCCCAAGTAGCC | SEQ ID |
| MYC | | NO: 20 |
| Enhancer | | |
| VI R | | |
| (homo | | |
| sapiens) | | |
| QPCR for | GCAAATGTAGAGTACAGGAGAG | SEQ ID |
| MYC | | NO: 21 |
| Enhancer | | |
| I F | | |
| (primer) | | |
| QPCR for | GTCTTCCAATTTCCAGGCAC | SEQ ID |
| MYC | | NO: 22 |
| Enhancer | | |
| I R | | |
| (primer) | | |
| QPCR for | GCATGTGTCATTTCCCCAG | SEQ ID |
| MYC | | NO: 23 |
| Enhancer | | |
| II F | | |
| (primer) | | |
| QPCR for | TCCGCATCCTTCCCTTTTC | SEQ ID |
| MYC | | NO: 24 |
| Enhancer | | |
| II R | | |
| (primer) | | |
| QPCR for | CCCTTCTTTCCTCCACTCTC | SEQ ID |
| MYC | | NO: 25 |
| promoter | | |
| F | | |
| (primer) | | |
| QPCR for | ACAAACCGCATCCTTGTCC | SEQ ID |
| MYC | | NO: 26 |
| promoter | | |
| R | | |
| (primer) | | |
| QPCR | CCCAGTAAGAACCATCCGAAG | SEQ ID |
| OTX2 | | NO: 27 |
| promoter | | |
| F | | |
| (primer) | | |
| QPCR | TGAAGGAAGCAAAAGCCAAC | SEQ ID |
| OTX2 | | NO: 28 |
| promoter | | |
| R | | |
| (primer) | | |
| QPCR | TCCCTAAAAATCCCCCTGTTC | SEQ ID |
| OTX2 | | NO: 29 |
| Enhancer | | |
| F | | |
| (primer) | | |
| QPCR | AACACTGTCCATTTCCAACTC | SEQ ID |
| OTX2 | | NO: 30 |
| Enhancer | | |
| R | | |
| (primer) | | |
| human | CCTGGTGCTCCATGAGGAGAC | SEQ ID |
| MYC | | NO: 31 |
| QPCR | | |
| primer | | |
| F | | |
| (primer) | | |
| human | CAGACTCTGACCTTTTGCCAGG | SEQ ID |
| MYC | | NO: 32 |
| QPCR | | |
| primer | | |
| R | | |
| (primer) | | |

TABLE 2
SOX11, HNRNPH1, and MYC Enhancer region sequences

| | | SEQ ID |
|---|---|---|
| Name | SEQUENCE | NO |
| MYC Enhancer | TTATTTTACCGGTCTGATGTGGGAATCAAATGGGATGATGCG | SEQ ID |
| Region, 5 KB | TGCAGAGGCCTTTGTAAACTCTGCCTCTCTCTGTAATGCAAG | NO: 33 |
| enhancer 1 | CTGCTATGGGTAGATGGCAGGCGAGTGGGGTCAACAAGGTTT | |
| | TCATCTCAACCCTTGCTGCACATGCAGCTCTGGGGAAAAATG | |
| | ATGCCCTGGAATCATCTGCAGAGTTGAGGGTAGGGCCTGTTA | |
| | TGGGGCTTTTTAGTTTTTAAGGCTCCTAAGGTGATGTTGGTG | |
| | GGCAGTAGGATTTGGAAACTGCTAGTAAACAAAAAAGTTACA | |
| | GATGTTACGAGAAAGCAGAACTTGTGGATTAGGGTGAGCAGG | |
| | CAGGAAAAGCCTTGAAGGAAAAGATGGGAATCTGAGAAGTAG | |
| | AAGGGACCCAGGCCAAGGGGAGATGGTAAAGAGAATGGAGTG | |
| | AAGACAAGGGAAAATGAGAAAGGGGAATGAAGGATAGAGGGG | |
| | GAGCTGGTACATCAATTAATCACAAAGTGGGTGGCACCTGGT | |
| | CTGGCGAAGGCTGCACCTCTTCCACGAAGCCTGGTGACTCCA | |
| | AATGTCTCTTATTGGAAGCTTCATGTCACCTTGTGCCACCCT | |
| | GTCTCCCCATTGCCATATTGTTTTATAATTGTTTGTGTGTGT | |
| | ATCTGTCTCCCTCAGTGGATCAGGGGTTCTTTGAGGGCACAA | |
| | ATTATTTCTTTCACCTCAGCATCTAGCAAGGTTCCTGGCATA | |
| | GACCAAGAACTTAATACATAATGTCTAAATGACAGGTATTGA | |
| | GCATCTGGTAAGGACTTATCTTAGGTCTGACTCTGCCACTAA | |
| | TTTACTGGGATGTCTACAACCCACTATTTTGCCTCTTTGGGT | |
| | TTCAAGGCCCCCCCCCACCCGCCACCAACATTGTGAAATGTG | |
| | AGTTCTCTAGGAATTCAAGTATGTAGGTAGCACTGTGTCTAG | |
| | AAAAAAATATTCAGTGAGGTATCAGGCTCAAATAATTAGAAC | |
| | ACATTAATTTAGGTCAGTGCTTCTCAAGCTTTAATGTGCCTG | |
| | CTAAGGTACATGGGGACTTTGTGAAAATGCAGATTCTAATTC | |
| | AGTAGGCCTGGAGTGGGTCTTGAGATGCCGCATTTCACACAG | |
| | GCTCCCAGATGATGTCAATGTGGCCTGTCAGCAGAGCTCACT | |
| | TGAGTAGCATGCGTCAATAGTACTTACTTTCAGGTGAGACCA | |
| | ATGCTGCTGGATTTTGACTCTCACTTGGAGTAGCAGGTGGTT | |
| | AGGCTGCGTTCGTATGATAACAAAATGATCTGCTGAAAATGT | |
| | CCCACTCCAGACCCTGAAATAATGATTTTTTTTTTTTTAATT | |
| | CTTGGCAGAGATACTTTGTTGGCTAGAATTATAGGACATAGG | |
| | TCAACCAAATCTGGGCTCAAATCCTGTTCCCAACGTTTCTTA | |
| | ATTATATGATTTTAGGCTAGTAACCTCAAAATCTTAGCCTCA | |
| | GTGTCCTATTCTATCAAATGGTATCACAGAACTTATCTCAAG | |
| | AAGTATAACTCATAGAGCATGATAATTTATATGAGGATGTAG | |
| | GTAACGCAATTAGCATACGGTGGGTGCTCAGTACATGCTAAT | |
| | TCTCTTCTTACTGACTTAGCAACACAGGAAGTAAAGAGATTT | |
| | CAGGAACTATAGGTTTGTTCTTTTCACATCGAGAAAGGAAGC | |
| | TTAAGTAGTATAGGAAATGCAGTACGTTTCACTTCCTTTCTT | |
| | CAGGGAACATAAAACCCCCAGGTGTATCTTAGAACAGGATCA | |
| | GTAAGAGAGAAATAGTAAAAGTGAAAAGTTCTCAGAGGGAGC | |
| | TGACTCACCACTCCTCAGGGAGAGGTAACCACCCTGTGAGGC | |
| | TTTGTGAGAAAGGTGGTCTGGGCAGCTTGGGGGAAATATGAA | |
| | GGAAAAGACAAAGATACAGGAACTCTCCGGCTCACAACAAAG | |
| | GCCTTCTTTCTTCAACACTCCATTTTCAACACTGAACTCTGA | |
| | CTCTTGTTGGACTTATCTTTAGACACATTCTATTTATTTGAT | |
| | TAGGGTAGAGGAATCAGAACAACTTCATATCCTGGCCTTCTG | |
| | AGAGGTCTGGTATGAGCACAGGTATTGTTATCTGACTTGATC | |
| | TTTACTGTGGTCCCTTTCAGTAACCTCCATAATAATTTCTTA | |
| | GGTTCCTAAATAACAAGCTCTAAGGGCCACTCCTGGGGTGAT | |
| | TTTCAGTGACTTCCCATCAGAATAGTTGTAATCAGCCTTTGT | |
| | CAATCTTCCACTGTCCCTTTTGCACCTGTCTTCCAATTTCCA | |
| | GGCACCTGTTAGGCCCAAGCTCACAGTAAGCTGTTATAATGA | |
| | CATCTCTTTGGGACCTCAATCAATAGCTCCCTGGTCAGGTGT | |
| | GTGCATGCTTTGTATTTAGCTCTCCTGTACTCTACATTTGCT | |
| | AATCCTAGCCAAGCGTTAAGAAAACAATCTGATTACAGATGT | |
| | AATCCTGTGACCATCTGCTTGATTTATCACTCCCAGAAAGAA | |
| | TTTGCATTTTGAAAAAGGAGCTTCTTAAAGTAAATGAGTCTT | |
| | TAATTTTGTGGGTTATTACAATAACTGCTCATTTTATATATA | |
| | TTGAGGGAATTTAAAAACATGCAAATGTGCAGGCCTCAAAAT | |
| | TTTGCATTTTAAAGGTCTCCATTAAGCTTTGAGCATCTGTAG | |
| | TTTAAAAAACTTTCCAGGTGATTCTGATGTACACCCAGAGTT | |
| | GCAAACTCCTGGCAATTGGTCGATTATGTATGGGACATTAAA | |
| | ACACATTTTTATTTTTTATTTTTGTCTATTCTTGACCTAAAC | |
| | ATCCATCAATTATCCATTGCTGTGTAACAACACACCCTAAAA | |
| | CTTCATGGATTAAACAAAAAATAACAATACTTCTCCTGAATC | |
| | TGTAGGTCGCTTTGGCTCAGCACGCAGCTCTTCTCTGACTAG | |
| | TAGTATCAACTGAGTCACTCCTTTGGCTGTATCCAGCTGATG | |
| | TCTGGGCTGGGTTGGAAGATCTGAGAAGACTTCGTTCACATG | |
| | GACAGTGCCTCATTCTCTTCCATGTGGCTGGTTTGTTTCTTT | |
| | TCACAGCATGGTTGTCTCAGGTTAATCAGATTTCTTAGATGG | |
| | TGGCTGGCTTTATGAATGAAAGCAGAAGCTGCCAGACTTCTT | |
| | AAACGATAAACCTGGAACTGGCATAGCATCACTTCTGCTATA | |
| | TGTTACTCACCAAGCCAAGTCAAAAGTCCACTCCGGGTTTAA | |
| | GAGAGTAAGAAGCAGACTATAAATTTTGGTGGGCAGAGTATC | |
| | ATGTGCATAGAGGAAAATAATCAATTCATGGCATCTTGTTTG | |
| | GAGACTATTTACTGTAACGTCTTCCCATCAATTTGCCAAATA | |
| | ATACGAACTTGCAGCATAACACCTTACCTCTGACGAATGTCA | |
| | ATCCTCATGATATAGGAGTTAAGAAGAAATTACTTAGGCAGA | |
| | TAGTGAGGCTACAGGAGTCCTTGGTAAAGTTTTCCTTTTAAT | |
| | GAAAAGCAGCCCCCAAATCATTTTCTTTTCTAACAAAGAGCA | |
| | GCCTGTATAATTAAGCTGCAGACATCGATAAGCAAGCTGGAA | |
| | GCTTGCACGGGTGAATGCCACCAGTTGTGCCGATAGGAAAAG | |
| | GCTACCTGGGACTAGGCATGTTCAAAATAGTGGCTCCATCTT | |
| | CCCTTCTCTTTGCCAAACCATGTGTACAGTAAGGAGCAGACA | |
| | ATATGGCCCCTGCTAGGCAAAGACATAATAATAAGATTAGGG | |
| | TGGGGCAACCAGCCTTTCCCTCTGCACTATGTAAACATCACG | |
| | CCTGGTCGAACCAATCTGTGAGCCTTACGTAAATCAGACATA | |
| | CCTCCTTAAGCCTGCCTATAAATTCTGGTGCAGTCCACCAAG | |
| | GGGTGTTTTTTTTTTCCCTTTTGGGAGCTCCTCTGTCTCACA | |
| | AGGGAGAGAGCTGGTCTCCTCCTTTCTCTTTCTTTTGCCTAT | |
| | TAAACCTCTGCTCCTAAACTCACTCCTCTGTGGTCTGTGTCC | |
| | TTAATCTTCTTGGCACAAGATGATGAACCCTGGGTATTTACC | |
| | CCAGACAATGATGCTGCTTCACTTACGCCAAACAAAAAAAGC | |
| | ACCGAATGTCCATATGGACCATCTAAGCATTCAAGACGATCA | |
| | ATGAGTCTCTTAAAAAAGATTTGAGGACTCATGGGTTTCCTC | |
| | CCTCTCTAATATTGCTGTAGATGTGGCTCAATTGATCAGTTC | |
| | TAACAAGAGGAACCTCAGGCAATCAATCACTCCGCTTACATT | |
| | TTTTTCAGTTTCCAAAGCCTAAGGGAGTTTTGTGGGAGGAGA | |
| | GTATAACCAAATGGAACATGGGCCTTGAGCCCAACAGAAACG | |
| | GTTTAGACTCTTAGATCTGCCAGCTGTGAGAGACCTTTGGTA | |
| | AGCTGATTCTCTGAGCCTCTGTTTTGGCTTCAGAAAAGTGAA | |
| | CGTGACATCTTCTTTCAGGCTGTTTGTGAAAACTCAGGGTGA | |
| | TGGTGTTCGTAGATGTCACCTGAACTGTTTAGCAAATGTTGC | |
| | ACCTTTTGCTACTAACATCTCTCTTTTGTAAAAGACATAACT | |
| | GTAGAAGTATTTAAACTTACAGGAAAAAGAAAGCATTTCGCA | |
| | AACACTTCTGGGACAAAGAGGAGAGAGAGAGAAAGCTTTGTG | |
| | TATATTTAATGAGTAAAAGCATATAATTTAAAATGTGAAAGT | |
| | TAATATCTTGAGTGAGTCACCCAAAATGAATTCTCTGATATT | |
| | GATAAGCATTGAACGTAGTTTAGTTAGTTTTCCAGAACAATG | |
| | ATTCTACCCTCCTTGGAAAATAGCTTCTAGGAAGTTTAGTGA | |
| | GGAAGCCTTGAGAAGGCGTCAGCACTCTAAGCCAGGTGGGCT | |
| | GTAGAGTTACAATCTGAAGCAACCGGTAGAGAGAAAAAAAGA | |
| | AGAATGGATTACAAAAGATGGGCAAGGTGCCTGGAATTTCAC | |
| | ACCTACATCATCTCCTTTGTGGTAAAGGGGAAATCCCAGGAT | |
| | CCATAGCTACCCCTGATATACTAGTATATCCTTGACCTATTG | |
| | GAGCAAAACTATAGAGAACAATGACTGCATTATTTCACAAGA | |
| | CACCCTTTGATCCAGTTTAGAACACTAGTTTTCCATTCTTCT | |
| | GT | |
| MYC Enhancer | CAAATCCAGTCTGCTGCCCCCTGCTTTTGTAAACAAAATTTA | SEQ ID |
| Region, 5 KB | ACCCGGAACAAGTCTGAAGTGTGATTCTTCAGTTTTATGAGG | NO: 34 |
| enhancer 2 | TGGCCCTATTTCTAATTTTGCTTCACTCGAAGCTGTCATCCA | |
| | AACATTTCAATTTGACCTTGTTCCACCAACACCCCATTGGCC | |
| | CTCCAGTGCTCCCACTCTCTCCACATCTCCATGTGGCAGTTT | |
| | CAAATCTCTGCCCACAGTTCCCTAGCTTCCTACCCCTGCAAT | |
| | AAGAGTCGGACTTTGCTTCGTCTCACTCCCCAAATCCCTGTT | |
| | GCCTCTGTGACCTTGCCAAATCTCCTCTTTCAGAAATGTACC | |
| | TCATCCTGCCTCCTTTCACCTGGCTCAAATGTGTCTCCTCCA | |
| | GGAAACCTTTTTGATGCTGGTATCCCTCTTCTGTGTCCCAAT | |
| | CACCTGCCATGCATTTACCATTGCTACCTGTCTAGTTTGCAG | |
| | GCAGGGCATGGTGGCAGAAGCTAGCCTTCTTTCGGGACACTC | |
| | ACTGGCCATCCCCACTGCCAAGAACACCTGCCCTTCCGGCTT | |
| | CTCTTCCCTAGCCCACTCTTATTCCTGAAAGTCTCATTTGAA | |
| | ATGTTACTGCCTCAGAAAAGCCATTCCTGAAGCCCCTGCCTA | |
| | TACTCCAAGGTCAAGGCCTGTGTCTTCACAGCCCTTTTTCAA | |
| | GGAATTTATTCCAATAAAATTAGATGGCTTATGGTGTGATTA | |
| | TTTATTAATGTCTTCCTACCCATTAAACAGACAGCTTCATCA | |
| | AGGCAAGCCAGCCATCTGTTCTATAGACCAAGTGTTTCCTCA | |
| | GACTTGGTCCACACCAGTAGACAGTTTTCTAGTGAATGAATA | |
| | GAGTTAACGTCACTCTAAATTAAATTAATCAAGTTGGAGCTG | |
| | GGTGCGGTAGCTCGCGCCTGTAATCCCAGCACTTTGGGAGGC | |
| | CGAGGCAGGCAGATCACCTGAGGTCAGGAGTTCAAGAGCAGC | |
| | CTGGCCAACATGGCAAAACTCCATTTATATTAAACATACAAA | |
| | AATTAGCCAGGCGTGGTGGTGGCAGGTGCCTGTAGTCCCAGC | |
| | TACTCAGGAGGCTGAGGCAGGAGAATTGCTTGAACCCATGAG | |
| | GTGGAGGTTGCAGTCAGCCGAGATTGCACCACCGCACTCCAC | |
| | CCTGGGCAACAGAGCAAGACTCTGTCTCAAAATAATAATAAT | |
| | ATTAATAATTAATAATAGTAATAATAATAAAGTTGGGAATGG | |
| | GTCAGTTAACAAAAGACACCAAGATAGAAACCATCGTCTCCA | |
| | CCAGTTAGCGACACAGAGGATGGATGTGTGCATTAGGCTTTT | |
| | CTCTGAGAATAGGATTTTGTCCTATCCAAGAAAAATGACAAA | |
| | CACCCATGAAGAGGGTTTTATTTTTTCTTGGTGGAAATGAAA | |
| | GTATTAGTTTTTCTAATCAAGAGTAAGTCATTTGTCCCCACT | |
| | TCCCCTGCAAACAACAAAAAAGAACCCATGATATTTGTTGAG | |
| | AACCAGCTTACAGCCTAATCCTGGCCCTGGAAGCATGCCATT | |
| | CTCTAATAGATACAATTAAGTTAAAAGACTGGGGTTGGTCAC | |
| | ATGGTTTCAGACAATGGCCCTATATTTAGGAGTTTTGTCCCA | |
| | AATATAATCAGATTCCAAAGGCCTGTGAGGATTTATTTATTT | |
| | ATGAAAATAAGGACCCAGTGCCTTCTGAAATAAGCCCTTTTA | |
| | GTGCAGCAGCCGTGTACACTGGGGCAGATTTACTCTGAAAAA | |
| | TAACAAGGCTTCTCTGACCCAGGAAAGTCACAGCCTGAAATG | |
| | CTTATTTTTGAAGCAGCATTTATTTTCTGCTGGTTTAGAACA | |
| | TGTTCCCCTTAGGAATAACCTCCTGGCTGCCCCAGAAATGCT | |
| | GATATGTGGATTGTGTAATGTCAGCTCTCCTGTGCGCCCTAT | |
| | CTGTGCCTCCTTCCTCGCAGCAGGGAGGCGTTCAGCCCGCCC | |
| | CTGCCCGTTCCCTGCCTTGCTCTTTTCTAAGAACTGGAAATT | |
| | ATTCCTGAGATGAGAGGAATAAAACATACATAGGACATATCC | |
| | AGTCTATTTCTGAAGCAGAACATGGAGAAAACCTAGCGAAGA | |
| | GATTACAAAAAAAGATAAAAGAAGTTTTCTTCCTAGTCCTCA | |
| | CTCCCAATCCCTTCCTGCCCAAGTTTATGTCTGCTAAGGGCT | |
| | CCTCTCATCAGCCCAACTTCCTGGGCTGCTTTCAGGATCAGG | |
| | AGAGAGAGGAGCTTCATAATATTTCCATGTTTACTTTTTAGG | |
| | CAAGGATAGGTAAGAAGGGCCCCATTCACAGAAGCACTGCTC | |
| | AGACAGGATAGCAGATGGCGGCTGGGTGGGAAGGAACGAAAC | |
| | CCTAGAACATTTCTGTCCTCATGATCCTCTTATGTGCCCTTC | |
| | TGATCAGAACATGACTCCTGATTGATTTTGAGGAAAGTTCCA | |
| | ATACCTCTGGCTGTCCGCATCCTTCCCTTTTCTCACAGAGTG | |
| | TGCTCAAGTGTGTCTGGTCATATGGTTTCTGATTTACACCCA | |
| | ACTCCTAGTTCCTCTTTCTCAGGTGGAGGCCTTCAAGACCTG | |
| | GGGAAATGACACATGCCTCCTTCAATGAGCAGTTGTCATCAA | |
| | CCATCAGTTAGCTCTGAGCTGAGTAGCTTAAATGTGTTAGGC | |
| | TGGGGAAATGGGGGCATTGAGAGAGCAAGGCAGGGCTTTGCC | |
| | TTCATTCTAACCATACATATATTCTCCAAGCAGCACAGGGCA | |
| | AGTCAGAAGATGTGGGTCCAAGATCTGGGCAACTTGGGCTTG | |
| | TGACTTCACTTCTCTATGACTTAGTTTTCCCATCTGTAAAAT | |
| | GGACTAAGCACCTCTCACTTCTTATTTTACCGGTCTGATGTG | |
| | GGAATCAAATGGGATGATGCGTGCAGAGGCCTTTGTAAACTC | |
| | TGCCTCTCTCTGTAATGCAAGCTGCTATGGGTAGATGGCAGG | |
| | CGAGTGGGGTCAACAAGGTTTTCATCTCAACCCTTGCTGCAC | |
| | ATGCAGCTCTGGGGAAAAATGATGCCCTGGAATCATCTGCAG | |
| | AGTTGAGGGTAGGGCCTGTTATGGGGCTTTTTAGTTTTTAAG | |
| | GCTCCTAAGGTGATGTTGGTGGGCAGTAGGATTTGGAAACTG | |
| | CTAGTAAACAAAAAAGTTACAGATGTTACGAGAAAGCAGAAC | |
| | TTGTGGATTAGGGTGAGCAGGCAGGAAAAGCCTTGAAGGAAA | |
| | AGATGGGAATCTGAGAAGTAGAAGGGACCCAGGCCAAGGGGA | |
| | GATGGTAAAGAGAATGGAGTGAAGACAAGGGAAAATGAGAAA | |
| | GGGGAATGAAGGATAGAGGGGGAGCTGGTACATCAATTAATC | |
| | ACAAAGTGGGTGGCACCTGGTCTGGCGAAGGCTGCACCTCTT | |
| | CCACGAAGCCTGGTGACTCCAAATGTCTCTTATTGGAAGCTT | |
| | CATGTCACCTTGTGCCACCCTGTCTCCCCATTGCCATATTGT | |
| | TTTATAATTGTTTGTGTGTGTATCTGTCTCCCTCAGTGGATC | |
| | AGGGGTTCTTTGAGGGCACAAATTATTTCTTTCACCTCAGCA | |
| | TCTAGCAAGGTTCCTGGCATAGACCAAGAACTTAATACATAA | |
| | TGTCTAAATGACAGGTATTGAGCATCTGGTAAGGACTTATCT | |
| | TAGGTCTGACTCTGCCACTAATTTACTGGGATGTCTACAACC | |
| | CACTATTTTGCCTCTTTGGGTTTCAAGGCCCCCCCCCACCCG | |
| | CCACCAACATTGTGAAATGTGAGTTCTCTAGGAATTCAAGTA | |
| | TGTAGGTAGCACTGTGTCTAGAAAAAAATATTCAGTGAGGTA | |
| | TCAGGCTCAAATAATTAGAACACATTAATTTAGGTCAGTGCT | |
| | TCTCAAGCTTTAATGTGCCTGCTAAGGTACATGGGGACTTTG | |
| | TGAAAATGCAGATTCTAATTCAGTAGGCCTGGAGTGGGTCTT | |
| | GAGATGCCGCATTTCACACAGGCTCCCAGATGATGTCAATGT | |
| | GGCCTGTCAGCAGAGCTCACTTGAGTAGCATGCGTCAATAGT | |
| | ACTTACTTTCAGGTGAGACCAATGCTGCTGGATTTTGACTCT | |
| | CACTTGGAGTAGCAGGTGGTTAGGCTGCGTTCGTATGATAAC | |
| | AAAATGATCTGCTGAAAATGTCCCACTCCAGACCCTGAAATA | |
| | ATGATTTTTTTTTTTTTAATTCTTGGCAGAGATACTTTGTTG | |
| | GCTAGAATTATAGGACATAGGTCAACCAAATCTGGGCTCAAA | |
| | TCCTGTTCCCAACGTTTCTTAATTATATGATTTTAGGCTAGT | |
| | AACCTCAAAATCTTAGCCTCAGTGTCCTATTCTATCAAATGG | |
| | TATCACAGAACTTATCTCAAGAAGTATAACTCATAGAGCATG | |
| | ATAATTTATATGAGGATGTAGGTAACGCAATTAGCATACGGT | |
| | GGGTGCTCAGTACATGCTAATTCTCTTCTTACTGACTTAGCA | |
| | ACACAGGAAGTAAAGAGATTTCAGGAACTATAGGTTTGTTCT | |
| | TTTCACATCGAGAAAGGAAGCTTAAGTAGTATAGGAAATGCA | |
| | GTACGTTTCACTTCCTTTCTTCAGGGAACATAAAACCCCCAG | |
| | GTGTATCTTAGAACAGGATCAGTAAGAGAGAAATAGTAAAAG | |
| | TGAAAAGTTCTCAGAGGGAGCTGACTCACCACTCCTCAGGGA | |
| | GAGGTAACCACCCTGTGAGGCTTTGTGAGAAAGGTGGTCTGG | |
| | GCAGCTTGGGGGAAATATGAAGGAAAAGACAAAGATACAGGA | |
| | ACTCTCCGGCTCACAACAAAGGCCTTCTTTCTTCAACACTCC | |
| | ATTTTCAACACTGAACTCTGACTCTTGTTGGACTTATCTTTA | |
| | GACACATTCTATTTATTTGATTAGGGTAGAGGAATCAGAACA | |
| | ACTTCATATCCTGGCCTTCTGAGAGGTCTGGTATGAGCACAG | |
| | GTATTGTTATCTGACTTGATCTTTACTGTGGTCCCTTTCAGT | |
| | AACCTCCATAATAATTTCTTAGGTTCCTAAATAACAAGCTCT | |
| | AAGGGCCACTCCTGGGGTGATTTTCAGTGACTTCCCATCAGA | |
| | ATAGTTGTAATCAGCCTTTGTCAATCTTCCACTGTCCCTTTT | |
| | GC | |
| MYC Enhancer | TTGCCGTTACAGGATAGCCAAAAACAGATTTTAAAAACAAAG | SEQ ID |
| Region, 5 KB | AGAATATTCTGTAATTCTACGACAGTCTTCATTAAGCTGTCA | NO: 35 |
| enhancer 3 | TTCATTTAGTCATCCAAATTTTGACCACATCCACGTATCACT | |
| | TTCACTGTTATTTAGTATTTAGTAAATATACTTTTATTAAGT | |
| | CATTTTACTTCTTTTTTGCTTAAATACATTTATTTATAAAGG | |
| | GAAATTAATGGAAAGAAAAAAAAAACACAGGAGCAAACTATA | |
| | GAAACGATCATGGCCTAAGAGAGAAAGCATTGCTCATGAATG | |
| | TGTTCAGGAAGAGCAGACAGGAATGGATCTTGGGCTTGGGGA | |
| | CCAGTTTTTCATTGTGTTGGCTCAAGACATTGGCATGAAATG | |
| | AGTGGAATGAGTAAGGGAGAGGAAGATCCAAGGCAGCAGGTT | |
| | GACTCACTGAAAGAAGTGAACTGCAATCTGAGAAGGAAGGTT | |
| | GGAGTAGAACGGGATGGCGGCTTTTGATTGAATGGTAGGGTG | |
| | GGGCACTGAAGCTAGAAAGAGGCCGTGACTGAAAAACTAGAG | |
| | CCAGGGGTTTCGAGAAACATTTAGTTTAAGGACATCACATAA | |
| | GGGTCAAAGCTGAAAAATTGGGAGAGCTTGAAGAGATATAAA | |
| | GCTGCTTTGGGGATGAGGGGATGCTTGGAAAGTTAGGGATGA | |
| | TTAATAGGATTTAGGAAATGGAAACTGCCATGTAGATGGAGT | |
| | CATGGTGTTACTGCAGCGGAGTAAGCCAGCAATTTGGAAGTG | |
| | ACAGATCTTTCAGGAAACATGGTAATGGAATATGTGTCTTCC | |
| | TCATTTTAAGTTTTATAGCATTTCTTCCTGGGATTACACTCT | |
| | GAGAGCAGGAATGCCCATCAGCAAGGGATAAGAAAAGTCAAG | |
| | ATATAAGTCTTGGGTGAAATAGTCAATATATCCCACCTAAAA | |
| | TTATTTTGCATATTGTATAATAGTACATGTATATATACCATA | |
| | TTTGGGGAAACTCTGCTTTAAAGCATTCTACAAATACATGGA | |
| | ATTACTTACAATTAGCTCTTGGAACAAACTTTATTATCCTTA | |
| | CAATTCTCCTCCATTCTTCCCTGGTAACTATATCCTGGTGTC | |
| | TAGGAACTAAAAATGATCCCATGGGCACAGTTGGCTTTGTGT | |
| | TTATATGGTATCTGGGGAGCCGCTGGAGGATTGGGCGTGGTA | |
| | AAATTATAAACTTATTGAAGTTCAAACTTAGAACACCTTTTA | |
| | ACATCTGGCAAATATAAACCATCTCCTATTTTAATTTTTGTT | |
| | TTTGGGCCTTTCTCAAAAAACTAAAAGTTCTTGCTCTAACAA | |
| | TCTTGGCTTAGATAGGAGACGGTCTGAAGTAAATGCGGTAGA | |
| | AACAGTTACATCATAAACTATTCCAAGACCTGTCCTAAAATG | |
| | GGAAATCTAGGAGATGGCTCGGTGCTCCATCTTGCTGGATTC | |
| | CCACACTGGAAAGCCAAGGACAAAGGAGGATTAAGTATTCCA | |
| | CGCATTATCTTGGGATTGGGGTTTCATGCTTGGGAGGCCGGC | |
| | GCCTGAAGTTTGCCTAAGAACAGACAGACAGCAGCAGCTCAA | |
| | AGCCTGATCTATACAACTTTATCTAGAGCAGGGTCATTTGAA | |
| | AGGATAGTGGTCATTGCTAATTTTGAGCATTTGCATAAACAG | |
| | GAGGAAACAATAAGGAAATTGTTTAAGAAGGAAAAAAAAAAT | |
| | CCTATAGCCCTCCCCTCCATTTCTCCCTCCCTTCCTTCCTTT | |
| | CCTGCAAATGACTGCTTGTTTAATACCAAACTAACCATTGTG | |
| | CTTTTGAGAATCCATGATTACTGCTTGGGCTGGAAGGTAGTA | |
| | AGATTTTGAGGCTAGAGGGGAGGTCAGAAGTCATCTAATTTA | |
| | GTCCTCTTACTTTGAAATAAGAAACTTACAGCCCAGTGAAGT | |
| | GAAATGATGATCCCAAGGTCACCCAATTGGTTAGTAGCAGAA | |
| | TCAGGCCTAGGTCAGTATTCCCTGCCTCCATCTGTTGTAAAT | |
| | CCTCTTGGACAGGCTTTGTGGCATGCTACCAGCAAAGCTTAT | |
| | TGGAAAGCATTTAAGAACGTTAGAGCGTCTGCAGATGTGATT | |
| | TTACAGGTTATTTATTTGTTTTACTGCAGATCATGACTTCCC | |
| | CTCCATCCCTCTTCTTGTTTCAGGGCCAGATTCTGCCCTTTG | |
| | ATGTTCTTTGTGGCATTCTTGTTGTCTTTGGCCCGAATCTGA | |
| | AGCAGGGCTTGACCCAAAGCTACTGGTGCCTTTGTTTGCTCC | |
| | CAGAGGACTTCCCCTCCTCCCAGGAGACTCAGAAAAATCAGT | |
| | TGTCTTAGAAATACTTTCTACACAAGATGCATCCTGCTGTGT | |
| | CTGTGTCATTGAATCTTTGAGTTGGAAGAGACCTCTGAGGTC | |
| | ATCAGTCCAACCCCTGACCCGGTTCAGGCATGTCTTCCAAAA | |
| | ATTCCCTGGCATATGGTCACATAGTCTTGTGGCTGGAAGGTA | |
| | CCTCAGAGACCAAAGAATATCTTGATTTCTTTTTGTAGGTAA | |
| | AGAAGCTTAGATTCAAAGGTGAAATGATTCATCTCCATGCAG | |
| | GTGGCAGAGCTAAAATTGTAATTCATGTTTCTGACCCTGAGT | |
| | CTGTAGCACCGTCCACTGTTCCATCCAACTTCTAATTATTCT | |
| | TCTAACAATTGTCCAGTGAATTATCCCTGTATTTATTTGTTA | |
| | ATCATAATTAATTTGCTAAGCCGCCCTTATTTTACCCTGTAC | |
| | TAATAGTTAAGGAAAATAGGCATTTAACTAGATAAAAAGACC | |
| | ACACAAGAATGGTCTTCATTCCCTAAAACAATCGGGGGTTTT | |
| | TTTTTAAGGTTCTTTTGGGTTTTTTTGTTTGTTTGTTTGTTT | |
| | TGTTTGTTTGTTTTTTGAGATGGAGTCTCGCTCTGTCGCCCA | |
| | GGCTGGAGTGCAGTGGCACAATCTCGGCTCACTGCAACCTCC | |
| | ACCTCCCGGGTTCAAGCAATTCTTCTGCCTCAGCCTCCCGAG | |
| | TAGCTGGGACTACAGGCACACACCACCACGCCTGGCTAATTT | |
| | TTCTTTTTTTAGTAAGACAGGGTTTCACCATCTTGGCCAGGC | |
| | TGGTCTCAAACTCCTGACCTCATGGTCCACCTGCCTCAGCCT | |
| | CCCCAAGTGATGGGATTACAGGTGTGAGCCACCGTGCCCGGC | |
| | CATTTTGGGTTTTTTTAATGCTGCTGTAACAAGTTATGAAAA | |
| | ATTTGGTTTAAGCCAAGACATATTTATGGTCTTAGTTCTGGA | |
| | GGTCAGATGACTGATGTTGCTGGGCTAAACTCAGGCTGCTGG | |
| | CAGAGTTGTTTTCCTTTTGGAGGCTCTAGGGAGAATCTGTTT | |
| | CCTTGCCTTTTTCAGCTTCCAGAGACTGCAACATTCCTGCTC | |
| | ATGGTGTGGCACCTGCCTCCATCTTCAAAGTACATTACTCTA | |
| | ACTCTGCTTCCATCATTTCTGACTCTCTCTCGCCTACTTCTT | |
| | TGCTTTATAAGGCCCTTCTGATTACATTTGGCCCATCTTAAT | |
| | AATCCAGAAAAATCCCCCTATTTCATGATCCCTAATATAATC | |
| | ATATCTGTAAAATCCGTTTTGCCATGTAAGCTGACACATTCA | |
| | CAGGTTCTTGGAATTTGGACCTGGATGACTTTGGAGGAGCAT | |
| | TATTCTGCCTACCACAGCGACTGTTGACAAAGCAGTCCAGCG | |
| | CTGTCACTGACATATTAAGATATAAAGTCGTTGCAGAAAACA | |
| | GATGCTTGAACCAAGTTGTGAGGGATGTGAAGCGTTCACTAG | |
| | CCAGGGCAGATAAAATAGAGAATGATGAGTAGAAAGAAGAGC | |
| | ATGTTCAGAGCTGCCCAGATATGAGAGTGAGCACATGGCACT | |
| | TTTGGGGAATGCTGAGTACTCAGGATGACTAGGAAAGACTAT | |
| | TATCCTATTAAAAATGCATTCTTAAAGTCTCAACAAGTTTCA | |
| | CACATAATGCAGTTAGGATAAGAGATGCAAAGAGATATCCAC | |
| | CTAACTCAAGGTAATATTGTAAATGGCACATTCATAAGGATT | |
| | GAAAGAGGAGGAGTATAATGAGAGAGGGAGGTTTGTATCTTA | |
| | AGTCTGCTGTTGAGGGACAAGAATAATCAGAAAGGGTCAAGT | |
| | GGTGTAAACTGCCCAAAATAGGAAGTCTGGAGAGGCCTCGAG | |
| | GGTAAGAAATATTTATTTTTAAAAAAGAAGAGTGCGACAGCA | |
| | CACATAATCTTTTGCGGGGAGGTGGGGGCTTAACTATGTGAT | |
| | ATCACATATGGGAAACATTCATTGTTGTCAATCAAATGCTAG | |
| | AAGCTGCTGAATTTAGGTCCAATGGTGACTCTCAAACTTCAT | |
| | CTCTGCACAGTAGCTTATTTAAAAATTTACTTGGGGTTGTCT | |
| | TTGGCTATACTTCATCATCATGATCTGTTCCAAGTTTACAGA | |
| | AGAGAGAATAAACAAATTTAAAAAATATATTTTTTCAATTTG | |
| | GAGGTTTTCCCTCATACTTTTTTTGCACCTTCTTCAACTTTC | |
| | TTTCTAGCAAGTACATATCCAAATGAATGAATGGTAATAAAT | |
| | ATAAAAATACAGATTTTATGCTAGGTCTTGAGATGTGAAGAA | |
| | CTGCAATATGTATTAGAGTCCTATTTCTGACTATACTATGAA | |
| | TAGATGGGCCCATAGTTCATTGGGTCCCACAAAGAACATCAT | |
| | ATTCCCACATGCTCTATTACCACAATTTGGATGGCAATTACA | |
| | TTTTTATCAGCAAGCAGTTATTGACACGAATTATGTCTCAGG | |
| | TTGTCTTATAAGCTCAAGGGATAGAGGAACATATAAAATATT | |
| | GCATTCTCAATGGATAGTTTTATATCTTAGACCTAGGACCTA | |
| | GAGATGACCTAGGCAATACTAATTTTTTCGGCACCCTCCAAT | |
| | TCACTCCCAAGTAATTTACTTGCATCCTTTAGCTCCTTGAAT | |
| | CCTCTTTACCCAAATTTTGTACAAAAGGAACAAGGAAGAGTA | |
| | GCAAGCTCATGGCAGGAGCTCAATCAATGGGTTTGGAACAAA | |
| | TAATGAGGACATTAAAGTAAAAATTTGGAGGTAAATTGCCTG | |
| | TGCTCACAGAGCTATTAATTGGCAGTGCTGAACTTGAACCCA | |
| | TA | |
| MYC Enhancer | TTATGATCATGATCTGTATGACCTGACATGACTTGATCTCGA | SEQ ID |
| Region, 5 KB | CTGCTGACTTCGATGTCCTCACTCTCCCTTCTGTCCCTGCTC | NO: 36 |
| enhancer 4 | ATTCACCATTGTCCATAAACACTGAACTTTCTTTTGTCCCTT | |
| | GAACATTTCAAACTCATTCCTATCCCTGGGCTTTGCTCATGA | |
| | TATTGTCTACTTGGAATAGCTCCCCTCATTTCTGCCTGGCTC | |
| | ACTTCTTCTCTGAACTAAAATAGTATCCTCTTCCTGGTTACT | |
| | CTCAGAAATATCACCCTATATTATCCTCTCAATTACATTTAT | |
| | CATTGCTTAAAATTATTTAATTTATTTATCTCTGTATATGTT | |
| | CATTATCTATCTCTCCTCATAGCTGAGAAGATAGTTGCTCCA | |
| | CAATATCCCACCTTTAGAGTTGTGCATGGCACATGATATGTG | |
| | CTTAATAAATATTCGTGGAATAAATAGACGAATGAAAAACAC | |
| | AAAAATGAATTAAAATCGTGGTAAGTACTGTGAATTAAGCAA | |
| | AAGGTGTCATGGAAGCTATAACAGTGGGGACTTGCATAAGAC | |
| | TAGGTAGTCACTGAAGGACTTTCTGAAGAAGTGACATTCAAG | |
| | CTGAGACCTGAAAGGTGAGTTAGAGTAAGTGCAATGCGGGGG | |
| | AAGAACTGTCCAGTCCAGGGAAGCAGCATGTGTAGGCGATCT | |
| | GAGGCCATGAAGAACTTGGCGCATTAGAGAAATGAAGGAGGC | |
| | CGGTATGGATTGAGTGGAGAGCATGGCATGAGATGAGTTTGA | |
| | AGGCCAGTAGAAGTCACTTATAGAAATGTGGTAAATGCTGAT | |
| | GTACAGTCATGCACAAAGGAGGCAATGCCTGGGGAAGGTAAG | |
| | CTGCTGGAGCTGGAGGAATGGAATTCAAGAAAGTCTTTACAG | |
| | AAGCAGCAATATTGAGGCTGAGAAGTCACCAGAACGATGGGA | |
| | ATGGAGAAGTGAGAGGAGTACAGCAGGGAGGCACACGCAAGG | |
| | GCAGACAAGTCTACAACAAGCTGCATATTCTGGGAACCACAG | |
| | GTATGGTGTATCAAGAAAGGCACAGCTAAGAGGAACACTCAG | |
| | GTGACTGTCTTGCTTGTCTTTATATCTCTAGCCCACTAGGCA | |
| | GTTAATCAATATTGGTTCATTGAATAAGGAAAAAAGGAAATA | |
| | CAGGAGACAAAACTGGAGAACTAGGTAGAAACCAAGCCACAA | |
| | GCATCCTTCTGTCCTTACTAAGGGTATGAAATTTTATTCAGT | |
| | AAAAATAAAACATAATAAAAAGTCTTATGATAGAACTGACAT | |
| | GATGAGATGGAAAACCCAGTCAATCTCGTATTTTCTCAGAGT | |
| | TTTAAAGAGGGGCCCACTTATTGATTAATTTAGTAAGAAGTT | |
| | TATCATTCATCTACTTGATTCAATGCTGCACACACTCACAAT | |
| | TTGCTCAATATAATTTTATTGTGAATCTTGAGCCTTGTGGTA | |
| | ATTTTACAGAATACATTAGGTTCTTTATTCTAGGATTGGGAT | |
| | GTGGAGTTCAATTCTAAGCAAAGTTTTCTATTGGGACAATTG | |
| | CAAATTTAAAACGATTAGCTTTAAATAGAGCATTGTTCCTTA | |
| | TCATGGATTATTTTGGTTCTTAAATGTCTGTCAAAGTAATAA | |
| | TAGGAGATTTGAGCTATTTTAAATATTTCAAAAGTCCAACAC | |
| | ATTTATCAATGATTAGGTTGCACATACTAATTAAATTTCAAG | |
| | GTTCTTTAGTATTAGCTAAAATTACACTAATTTCAATGTGTG | |
| | GTCACGCTGATGAGCATGAATGACAGTCTTAAGTGATGATAG | |
| | CCTCTTTGCTGAGCAGTGTCCCTGTGTAAAGATCCACAACTC | |
| | TCCAATCATCTTTGGAGGACCTGAGATCAATTTGCATGAAAT | |
| | AGTATTCATTAGACATTTCTGGGAAAGAACTCATCCTAAGGA | |
| | AAAGTTCAGTCAAGTAGCTCAAGTTCAGGCAAATGGCAATGA | |
| | AGGACTGTTGAAGGGAAGGTAGGATTTATAACACAAAACTAT | |
| | CTGGGTGGTCTTAGACCTTAGTCACTGAGAGCCAGGGCTTTG | |
| | GAATGTTACATATCTTCACTTGAATCCTAGCTTACCAATTCT | |
| | TTCTGGGTAACTTTGGACATTGGCCCAATCCTGTAGAGCCTC | |
| | AGTTTTCTCATTTGTGAGGTGGGAATCATCATGGGTACTATG | |
| | TAAGTTTGTCTTAGGGAGGGGGGCTATGCATGAAAACTATTG | |
| | CTCTAAGTTCTCCATAAAGAAAAAAGTGGCTATGTTCTGTCC | |
| | TACTCTGTCTAGGCCTTCTCAGGCCTTCAACCATTGCCCATG | |
| | CCCACAGCCCAGTCATCATCATCCACTTAGTTGCAATGTGAC | |
| | TCCACGCATGTCCTGGCCAATTCAGTAACCTCTACACAATGC | |
| | AGCAAGATGGCACTGGCAGTATGGCACCTAGATCCTGGGTGT | |
| | TGGAGCCAGGCAACATGGCTTGCTAAAGGCAATGTACAGTAG | |
| | GCAGGATTTGAAGACAACTGCTAATAACCTATACCCTTTTAT | |
| | TATCCCCTCCTGTTAAGTGTGAAATGACCTGTTAATATGATG | |
| | ATATGTGGCAAAAGGGATTTTATAGAGGTAATTGGGGGCCCT | |
| | AATCAGTTGAGTGCACTGAAAGGGAGATTATGCAGGTGGGCC | |
| | TGACCCAATTACATGAGCTCTTTAAATCTGGATCTACAGATT | |
| | AGAGACAAATGAAGTCAGAGACTCAAAGCATAAGAGATATTC | |
| | AACATGATAGAGACTCTCCACTGCTGGCTAAGACAGAGGGAG | |
| | CCATGTGGCAACAAATAGAGTGTATTTTAGTGGCCAAGAATG | |
| | GCAGCTTCAGATGGCAGCCAGCAAGGAAATGGGAGCCTCTGT | |
| | CCTACAGCAACAAGGAACTGAGTTCTGCCAACAAACTAAATG | |
| | AGCTGGGGAGAGGATCCCTCGTTCCAGATGAGATGAGCTTGA | |
| | GTTTGGTAAACATTATGATTTCGAGCTTGTGAGTCCCTAAAC | |
| | ACAGAGCCTAGCTAGGCCATGCTCAGACTGCTGACTCATGTA | |
| | AACTGTAAACTAACAAATAGGTGTAATTTTAAACTGCTAACT | |
| | TTGTGTGAGTCTGTCTTAGAGCAATAAAACACAAGTACAGAA | |
| | TAATTGCCTACATGGAGTTTCTGGGTAGTTAGGAGCATGAGA | |
| | GAATCTCTCTGGGGCTTTCTGGCACCTGGAAGGCCTTCCCTA | |
| | TGGACTGGCAGAATACTTCCTGTTCGAGCAAGTCTCTCCAAA | |
| | GCAATGGGGATCATGAAAAACTGAACTCATTGACAATCCCCC | |
| | AATCTGCTTAGTTGTGTTAGACTTTGTTAAATATGCATTTAT | |
| | TATGCATATCAAGTATATATCAGTCATGCAGCAGACAGATAC | |
| | AAAACTTACACTGGGTAACAGAGGAGAGTTTTATAAATGGAC | |
| | TCTATGCAAAGGTTTGGAGACATACACATGGGAGTGTTCAGT | |
| | ACCCCAAAGGTTAGCAATAGCAGTGAACTGTCAGTACCCCTA | |
| | GGGAAAAATCTCCTGTTTTCTAGATCTCTTTCCCAGCTGACT | |
| | TACTTTGATGGTATATGATTCCCAACAGCTTTCTGAGTAAGG | |
| | CTGTCTGATAGACAACAGTTTTGGGAAACTTGAGTGTCTAAA | |
| | AATGTCTTTGTTATCTCCTCACACATTGGACTAAGGGTATAG | |
| | GATTCTAGGTTGGAAACCATTTTTGCTCAGGTGTTTGAAAAT | |
| | ATTATTCCATTGTCTTCTAGTTTTTGATGTTGCTATTAAGCC | |
| | CAATGTCATTGTAACCTTTCATGCTTTAAAGGCAATTTTTTA | |
| | TTCTATTGCATCGCTTAGTATCTTTTTATTCTTGGAGTTTTA | |
| | AATTTTGTCATGATGCCCCATGATGTGGATCTTTCTTCTTTC | |
| | ATGACGTAGGAAACTTGTTAAATCCCTTTAGTCTAGAAACCC | |
| | ATATATGTTGGTTCTGTGCATATTTTTTAAAAATTCCTAGAT | |
| | AATTTCCTCCCATCTAATCTGTCATTTTTTTTTCCTGGAACT | |
| | AATATTTGTTAGATATTAGACCCCCAGAGGATTGTCTAATTT | |
| | TCTTACCTTTCTCCAGTATTGTCTATCTCATCAACTTTTATT | |
| | TTTTCAAATTTCTGAAGAGTTCTTCAACGCAGTATTCCAATT | |
| | ATCCTCCCTTTTTCTTGACACTTTAATATTTGCTTTCACAAC | |
| | AATTCCAAGGATATTTTTATTTTGTAAATGCTTACCTTGTTC | |
| | TCTGTTTTATATATTCAAGATTTTCCTCAATTATTTTACGAT | |
| | TTTTGGCACTCCACTAATAACTAGAATGGAGCACTGGAAAGT | |
| | TAATTAGAGGTGCTGTGTATGTGACTGAGGCTGTCAACTGGT | |
| | TGACCTCACCATAGAGTGATGAGAAGAGGATCTGCCATTTTA | |
| | TTGTGATAGTATCTGCATGTTTGTTTTCCTCCTGAGACGGAT | |
| | AGTCTCCATGGAGAAGAATTCTCCAGTGTCACAGTTGGAGAA | |
| | TAGAGACCTGAATGACAGTGTCAGGGAAAAATCATAAATAGA | |
| | ACTTTTCAAAATGTATATTTTGACTTAATTTTACATTACAAA | |
| | AATGTAATTAAAATTACATTTTTGATATATCTTCTTGTATCC | |
| | ACCCTCAGATAGTCCTAGTGTCTCCATGTTCTCTGGTCTTCT | |
| | GTAATAGAGCCTCTGTAGATAGTAAGTGTTGGGGAAGGAGTT | |
| | ATTTGGCTATTAGGACTAGACAGTGAATCTGTGGTTCTAACT | |
| | CTTCCTTACACATATTGTCAATCAACCCTCTCATTTTTCTCC | |
| | CCTTTCAAATACACTGGATGCCAATGAGCCATTATCTTTTCT | |
| | GGGTCTTCTGATGTTAACTAGATTTTGACATCCCTAATATGG | |
| | ACTTAGTTTTCAGCTTGCTCTGCTCTCATCAGTTGGCTACCT | |
| | CTATCCTTTTGTTTCCCATAGTTAACAATTTAGTTGACATCT | |
| | CACAACTGATGTCATTACCTTTCCAATCCTCTTTGTTCTTGT | |
| | AAGTTTATATCATTTCTTCATTCCTTTACAATCATTTTATTG | |
| | GTTTGCATAAAAGAACGGAGTAAAAGCATAGATTCAAGCCGC | |
| | CA | |
| MYC Enhancer | GGAAGGTGAGATTATTACTGACTACATGTGGAGAAACTCATT | SEQ ID |
| Region, 5 KB | TGTAAGTGGCTCAGTGGCTGTTCCTAGTTACGCAGTAATTCA | NO: 37 |
| enhancer 5 | GTGGCAGGGCTGGTCTCCAAACTTCCTGCTGCAAGAAAGATC | |
| | CACAACTGACCCAGCTGATTGTAAATCATTGAGAGCAAGGAC | |
| | AGGATCTGATTCATCTCTGGACTCAGGGAGATATTCATCATT | |
| | CACAACTGGCAATCATGCTCTCCAGCCAACCAAGATGGAAGC | |
| | CTCCTAGAACATTCTGGTGCGCACCAGCTGAGAGGCAGCTCT | |
| | GCCTGTCTTCCTGAGTGCCTGAAACACAGCTGACACTCGGCA | |
| | AATGCTTGTTGAATGGAATTAAATTTCATATCAAGAGAGTAT | |
| | GACTGATTAAGAAAGAATACCTCAAGAAAATTGCTACTGACA | |
| | AAAACGTTTTTGTAGTTCCCTATTTTCCCTTTATATTAATAT | |
| | AAATTTAATATTATTTATTGAGCACCTACTATATGCCAAGCT | |
| | ATATGCTAGAAGCTCAATATATTCCATGTAATTAAGGGTTGA | |
| | AAATGAAAATTTTACAAATTCATTATCTTTGGGCTCCTTTTC | |
| | GCAGTGGATGGATCCAGAGAGGACTTGTGAATGAATGAAGCT | |
| | TTGATGGCTGGAGGTCATGGATTTCTCATTTAAGATAAGAAG | |
| | TGGCATCCTATGCCAGATGGTTTCCTTCCTCTGCCCTTGCAC | |
| | GTGTTTTTTTTTTTTTTTTCCTCTACCAGGAAGGGCACCACG | |
| | ATTCTCTCTTGTGTTTGGCACATTCCAGTTTAGTGCTCACAG | |
| | CTCTATTACGGCATCCAGTACCCTGTAAAGTCTGCTTTCACC | |
| | CTCTATCAGCTACTCTCAATCATCTCTCCTTTTTGTGATATT | |
| | TTTAAACTTTGTGCATGTCCATCAAGACCCATAAGAAAATGT | |
| | GGCATATTATCAGTATAAAGAGCTATCTTTCCCAGGAGACTA | |
| | AGAATTCCTCAAGGGAAGAAGACTTCCTCCTCCTTGTCCTGC | |
| | TCCTCCTGCTCCTCTTCCTCCTGCTCCTCCTCCTCCTTCTTC | |
| | TTTCTTCTTCTTTCCTTCTCAAGAAGGAATGATAATTCCAGT | |
| | GCCTTTCGCTGACACATTCTAGATGCTTTCTGTCAACTGAGT | |
| | GATGCTCACTTCAGCTCCTGGACCTGAGGTGTCATCTGCCTT | |
| | CCTGCCCATGGAGATCTTTCACTGGATATCTAACTGCTGCTT | |
| | GTGTGGCCTCTCAGGCAGACATCATGCCTCGTTCATTTTTTG | |
| | CATACCTATGTAGTACTTATACATCTATCGCATCTTTCATCT | |
| | CAAATCCTCATGGCATTCCCCCTCTGTTTGTTATCATTACAC | |
| | CCAGAATGGCTCTTTGAGGTCTCACAGTCATTTTCTTCAAGG | |
| | TGGAGAAACTGTGGACCAGGAGAAATTCTGGGTCTTCTCAAT | |
| | CAGTCATAGAAGGCTGAAAATGCAATTAGCCATTCTCAGGGC | |
| | AAATAGAATCTCTGAAAGGCATCAAAGGAAGCTCCTTCCTGC | |
| | GTCTTCCTTTCCTTGTCCTTCTTCTTTTACTCTATCTTCTCT | |
| | CCCTCCCCACTCATAGATATATAAAACTGGAAAGGATTAAGA | |
| | AATCCTGTCACTTAAAACTAATATTTGACAGATGAGGTTAGA | |
| | GAAGATGGGCAGATTGGGATTCTAAGTCCTTGATTGTCTTCT | |
| | TAAAAATGATACCACTGAGACCCCTTATGACTTTTACTTGTG | |
| | TGGATAGGGTTAGGCCCTGCCACCACTTCCATTTATCTTATT | |
| | TCCTGAACTGGCTGCTTGGCCTTGTCTTGGAGAGGCAACACG | |
| | TCCACTGGTAACTCCTTCCATCTCTCTGCACATGCCACCACT | |
| | CACTGCTTGTTTATCAAGAGGTAGAAGAATTTTACTCAAAAT | |
| | TGTGGCTTAAAAATGAAGCAAAATAAAACCTTGATTACAGAG | |
| | ACATATTCAGACATTTGGATTTGTCACTAGGAGTAGCGGATC | |
| | AGCATTATGTGCTAATTCCATGACATCATGCATACGTTACAT | |
| | TGATAAAGAGACACTGATATTGGGTGTCTACAGTATGCCAGC | |
| | TAATTTATGTATAATTAAGTTAAAAGGCAAATATAATTCCCA | |
| | GTGTATAGATGAGGCAATTCCAGCTGAATAAGTTTGAGGGTT | |
| | CTAATGTAAGGGCGAGGCGTTTGGGCTCTGGAGTCATAAAGA | |
| | CACATTAGTACTTCCCCATCTGAGCTCTGCTACCTCCTCACC | |
| | GTGCAACTGTACGCAACTCATCCTTTCTGTGCCTCACTTTCC | |
| | TTGTCTGAAAATGTAGATAAACAACGCTTTCCAGATCAAGTT | |
| | GTTTTGAGGATTAAATCGAAAAATTAATGTAAGAAGGTTTAG | |
| | CACTGTGCCTAGCACATGCCAAATCCTTCTTAAAAGTGATAA | |
| | TTGCCCCAGATCATGGAGCTGCTAAGTGTGATGCAGAGGACT | |
| | TTGAGACAGGCAGAGTTTTGTTTCTACCCTTGCTTCTGCCAT | |
| | CTTGCCTGCAATGTTGGGTTGGCATGAGAATTATAGATAATT | |
| | TGAATAAAAAACCTAGCACAATGACTGCCCTGGAATAAATGG | |
| | AAACTCCATTCTTGCAGTTGGCTCAGGGTTAAAAAAAAAAAA | |
| | AAAAGAAAAAAGAAAAAGAAAAATTGGAGTCATTTTTGACCT | |
| | TTCTCTCTCATACCTCCCTGATACCATCAGCAGATCCTCTGG | |
| | CTCTGCCTTCAAAATAAGTCTATGATTGGATCATTTCTCACC | |
| | GCCTCTGCTGCTGTCACTACCTTGTTTGGATTACTACAATGG | |
| | CCTTCTACCTGATTCCCCTTGCTCCACCATCCCCAACTTTCA | |
| | GCCTAGTTTCCACTTAGCAGCTACAGTCAGGTTGGAGAATGT | |
| | GAGTTAGATCAGTAACTCTGCATCCTCCCACAGCAGCTTTGC | |
| | AATTTGCTCAGAGTAGAAGCTGAAAACCCAATAAAGGCTTAC | |
| | AAATCCTCTGTGAGCTGCAAACCACACCCAACCCCAGCTTCA | |
| | GCTGTCACTGTTTGCCACTTTTATTTATTGGCTTTTGTTTTG | |
| | TTCATCAAACACCAGAATACCTTAAACATTTGCATTTGCTGT | |
| | TGCAATGCTCACGCCTCAGATATTGACATGACTGATTCCTCT | |
| | TGTCTCTTGAATTTTTCTCAAGCGTTGCCCTATTGATGATGC | |
| | CTTTCCTGAACTCCCTATTTAAAGTTATACCTGGTTTCCATC | |
| | GCCATCGCCACCGCAACCATGCTGAGCTCTCTTTCCCACTTT | |
| | CTTTGCTCTATGTTGCGCCTACCGTGAGCTACCCCTTGAGTG | |
| | TATGAATAATGATTTGATGGTTTCTACTACTTATTGTCAAAA | |
| | TATTCTAGTATCTAATTCTGAGTTGTTTGGCTGTGAGACTGA | |
| | ATATTCTACTTAATTTTTCCTTGGCAGTGAAATATGAACAAT | |
| | GATACATATAAGGTTCTGCATGCTTGTACTTGGTCACTTGTT | |
| | TTTTGAGAAGTAAATAAAACACCATTTCCAAACAGTCTAAAC | |
| | AGAGTGGCTGACATACACCAGGAGTCAGTTAATAGTAATTGT | |
| | TTTTCATTTGATTAAATGAATTCCATGGCCAGGCACGGTAGC | |
| | TCACACCTGTAATCCTAGCACTTTGGGAGGCTGAGGGAGGCA | |
| | GATCACAAGGTCAAGAGTTGGAGACCAGCCTGGCCAACATAG | |
| | TGAAACCCCTGTCTCTACTAAAAATGCAAAAAAAAAAAAACA | |
| | TTAGCCGGGCATGGCAGTGTGTGCCTGTAATCCCAGCTACTC | |
| | AGCAGGCTGAGGCAGGAGAATCGCATGAACCAGGAGGCGGGG | |
| | GTTTTAGTGAGCCCAGCTGAGATTGTGCCATTGCACTCCAGG | |
| | CCAGGCAACAGTGTAAGACTCTGTCTCAAAAAAAAAAAAAAA | |
| | AAAAAAGAAAGAAAGAAAAGAAAAAGAAGTGAATGTGAATTC | |
| | CATTCAATTTGGCTTGACAATGATTTATTGAGTTATATTTTA | |
| | AACTTCTTAATAGAGTTTCCCTTTTAAAAGGCATACCAATTA | |
| | TAAATACAGAATTAGTTGTGAAAATGAATATTTACTCAGAAT | |
| | AAGATATCACAAGTTGCACAATGAACTAAAATTAACAAATAT | |
| | AATTAATGTCAGAAAAATCTACATAGTAATATTTTAATTAAT | |
| | TTCTTTTAAACACAATTGAATAAAACTTCTTTGCTACTTTTT | |
| | TGGGCTGCCTGTCTTTTCAAAGATCTCCCTGCCAGATGACAG | |
| | TGGTGTCTCAATATCATTTTCTTTACAGGGAATCAGAAGTTA | |
| | ACTCAGTCTTTCCTCTAGCATGGTTGACTGGAATTTGTGTTT | |
| | ATTATTGATAGTTGGGATGCATAAAGCATATTACCTCACCCA | |
| | CAGGCAGTCATGCTATTTGTAGTACTGCTAGAGATGTGTGCC | |
| | CTATAAATATTGGACTGCTGATACGTTCTATTTTGTGATTGC | |
| | ACTCGAAGAAAATAATGCATGATGCATTTATAATTGTATCTG | |
| | CTGCACAGTCTGCACATCCTGTGAGAAAAGTCGCTCCGCGTC | |
| | GACTGGGCATCCTGACTTTCTTATTGCTTTCACTGTTGATCC | |
| | TGTCCCACTTTACCAAATCAAGCCTTGTCTCTTACACACAGA | |
| | CTCATACTACAAAGGGTTGTGCTGAGATGTGAAAACTTCAGA | |
| | GGGACTATTTGAAATATTGTTTAAAAAAGCTTCATCTAATCA | |
| | AGATGCCATAATGTCCTCTCTCTCCACCCCCATATATGACAA | |
| | AGAGGTGTCTTTCTAATGAAATTTATTTTTTAAGTTAAAATA | |
| | TTTAGAAAATGGATAATCTCTTTAGCTCAATTGTCTGTTCAC | |
| | ATTAATTGCATTAACATTTATATATGTATGAAGAAAAAATTT | |
| | AAACTCATTAAACTTTATGATCACAGAAAATTGAATGTACAC | |
| | ACATATCTATGTGCATAATTATATGTACAGTATGTGTATAAT | |
| | TTTGTTGTAGAGATGTGTGTTGGAGTAATCAAAGAAAAACCC | |
| | TAAAGTGTGGAAAATATGTGAATATAATATTCTCTGGAGAAA | |
| | GA | |
| MYC Enhancer | TAAGCTCAAATCTTAGTTTTGCCTCTTACAAACTATGTGACT | SEQ ID |
| Region, 5 KB | TTGGGATGATTCATTTATTTCATCCTTCTGGACCTCAGTTTC | NO: 38 |
| enhancer 6: | TTTATTTGTAAAATGGGAAAGCAGCCACGGAGAACACAAAAG | |
| | CAAGTGAGTTTGGCCATGTTTCCATAAGACTTTACTGATGGA | |
| | GATTGAAATTTGATTCTCATATACTTTTTACGTATCACAAAT | |
| | AGTATTCTTTTAATTTTTGCAACCATTAAAAAAATGTAAAAG | |
| | TCATTCTTAGCCTCTGGGCAGGCTGCACTAAAACAAAGGGAG | |
| | GCCAGATGTGGCCTGAGGACTGCAGTTTGCTGAAACTGCTCT | |
| | AGTCATTTCTCTCTTCCTGGTCAGGGGCTGCAGAAGCTGAGT | |
| | CCGTTCTTCCAGAACCTTTTGTCTTCAGAACTTATCACTCAA | |
| | GTCTGGGAGGAAGGCAAGAGGCCCTCCCTTTTCTTTGTGCTG | |
| | TTGAGGCAGCTCCGGCCTCCCTCTGAGAACTGCACAGGGAGA | |
| | GAGGCCTTTTCTGGCTGCTTTTCTCCCAGTCTCAGCTCACAC | |
| | CCAGGTTTTTGGCATTGTGTTAGATAATTTTGCTAGTAGATG | |
| | ACTGGTCCGTACAACTCTCATTATGGGGGCCTCTGTGCATGA | |
| | AGATGTGAATGTCTAAAGTGAGGGGAACTTCAGGCCAATTAT | |
| | TAAGCCTGGGAAAGTTCACGCATCTGAAAAATTATGCATAGG | |
| | CTATGTATATGCACATCACCTACTCCACTCCTATTATACACA | |
| | CCCCAAGAACATGGTAAAAACATGCAAGAAAAAAAAAAAAAG | |
| | AGCCAGACTGGCCAGGTGTGGTGACTCATGCCTGTAGTCCCA | |
| | GCACTTTGGGAAGCCAAAGTGGGTGGATCGCTTGAGCTCAAG | |
| | AGATGGAGACCAGCCTGGGCAACAGGGCGAAACCCTGTCTTT | |
| | ACAAAAAATACAAAAAAGTTAGCCAGGTGTGGTGGCGCACCC | |
| | CTGTAGTCCCAGCTACTTGAAGGGTTGAGGTGGGAGGGTCGC | |
| | TTGAGCCGAGGAGATGGGGGCCGCAGTGAGCCGTGATTAACT | |
| | GCACCACTGGACCCCAGCCTGGGTGACAGAGGGAGACCATGT | |
| | CTCAAAAAAAGAAAAAATAAACCAACAAAAAACCCAACCACA | |
| | ACAAAAAACCAGCCAGACCTTTCTTTTCATTATGAAAATGCT | |
| | ACTTCTTAAAACGTCTGCATCATTGACAGTATCTCCTTTATG | |
| | TCCTGGAATGCGAGAAGGTAGCACACTGTATATGCCTTTTCA | |
| | GGGTGGGAGGGGGAGAAAGAATGGAGACACAGGCCCAGACGA | |
| | GAGACCTTTATCATTTCCTCTAGGAATTTTTCTTGGCTCTCT | |
| | AGGGCTGGGGATTGACAGGGATAATGAATTCATGGCTCAGCT | |
| | GGCAAATCCAATTAAGCTTATTAACAGGGTGGCCTTGCCTTG | |
| | GGCCTGCTCTGTGGTGGGACCTTGGCAGAGAAGGAAGTGCCT | |
| | TCTGTGCCTGGGGCATCTGATCCCCTCTTAGGCAGCCGTGGA | |
| | AGCTGCAGTTAGGGCATTTCAAGGGACAGCAGGACTGCACAC | |
| | TTATATAGAGTCTGTGGTGCATAATCTAGAACTAGGATGACC | |
| | AACCATCCCAGTTTTCCCAGGACCAAGAGGTTTTTCTGGAAA | |
| | AAAATAACAAAGTCTAGGAAAAACTGGGACAAGTTGGTAACT | |
| | TTAAATCTTGCCTCAGGAAAAAATGAGGCCCATTGTGTCCTT | |
| | GAAGGGATGTAAGTAATCTCCTATGTAGATCCTCTAGAACAG | |
| | GCATCAGCAAACTTTTTGTAAAGGACAAGATAGTCAATACTT | |
| | CAGGCTTTTTCGGGCCACATGGTTGCTGAGTCACAACTACTC | |
| | AGCTTAGCTGTGGTGATGTGAAAGTAACCATAGACAGTATGT | |
| | AAATGAATGAGTGTGGCTGCGGCTTCATGAAGTGTTATTTAT | |
| | AAAAACAGGCCAGATTTGGCTGTGGGCTGTACAATGCCAATG | |
| | CTTGCTCAAGGAGGTATCCAGTTTTTCTACCTCTCTGTATGC | |
| | CACACATACATACACAAACAGGCACACCCGCTGTAACATTGT | |
| | TTGGGAAGCAAGACAGCCCAGTGGTTATAGAGGGCTTGAACT | |
| | TTGGAGTTAGAAAGATCTCAACTTGAATCTTGGCTTTCCCAC | |
| | CTGCTAGTTGGGTGACCTTGAGGATCGCATCTCTGAGCCTCC | |
| | GAGCTTCAGTTTCCTCATCTAGAAACTAGGAATAATACAAAT | |
| | GGGATCGCCGGGCACGGTGGCTCACGCCTGTAATCCTAGCAC | |
| | TTTGGGAGGCTGAGGTGGGTGGATTGCCTGAGCGCAGGAGTT | |
| | CAAGACCAGCCTGGGCAACACGGTGAAACCCCGTCTCTATTA | |
| | AAAATACAAAAAGTTAGCCGGGAGTGGTTGCATGCACCTGTA | |
| | ATCCCAGCTACTTGGGAGACTGAGACAGGAGAATCGCTTGAA | |
| | CCCAGGAGGTGGAGGTTGCAGTGAGCCGAGATAGAGCCATTG | |
| | CACTCCAGCCTGGGTGACAGAGTGAGACTCTTCTCAAAAAAA | |
| | AAAATAAATAAATAAATAAATACAAATGGGATCTGCCTGCCA | |
| | AGATAGAAGGAAAGGGGATTTGGACTTCATCTCTTGACTGGG | |
| | GAGGTGTCAGAGAACATGCAGTTGTCTCTAATTCACCACAAG | |
| | AGCGGGCCGGGAGGGGTGACAAGGTTATTCAGCAATCGATGC | |
| | CTCTGTATCTAGCTCCCAGCTAGTTGCTGAAGCCAAAACACT | |
| | CAGTAAGATGACAAATAGGTGTTTTCCTTGTGGAATTCATGG | |
| | TCACATAGAAAATTTAGATAGCTAAACAGCCAGTTATAGTAA | |
| | ACAGGGTTGAAGTCCAGATAGTGAGAAGGCAAGAGTGAAGGA | |
| | GGCTTCTGATTCTTTAAGAGAAAAAAGATTCGATGAGGGAAA | |
| | TACATCCCTTTTGCTGCCTTAGAGAGCAAAGAAGAGCAGCTG | |
| | CTCAGGAGCCGCTAGGGTTGGTCACATGTGCAGGTGGCTTCT | |
| | CTTTGTGTGAAACTGGGTGAGGCTGTTTGCTGCAGGGGTGTG | |
| | GGCTCAGGATGTGTATGGATATGTTTGTGTGCCTCCCGGTAG | |
| | ATAACTTGTGGGCTGATGACAGAGAAAGGGCAGCTCTGATGT | |
| | AACAAGAGACATGCTCACATCTGTGACTTGAAGAAGCCGAGA | |
| | CTTTAGGGCCAGAAGGGTCTTAGAGATGCCTCATCCAATTCC | |
| | TTGTCCTCTTCTCATTTTACAGGTGACTAAATGGAGGACTTG | |
| | TCCAAAGCATCTCAGCTAATTGGCCCTCTGATTCCTCATCAA | |
| | AATAACATATTTCACAAGCGAGGCCAATTAGATTCTGGGTGA | |
| | ACTCAGTGATTGAGTTCAGATACTGTTCACTAGCTGGCCTCA | |
| | CCTGATTTGGGGTTGACAGAAGAAGGCAGGGTGAATGTTGAG | |
| | AAGACAAGAGTGTATGCTCATCAGAGAGGACTGGGCACAGTG | |
| | CCAGGCACAGTTCTCAGAAATGGCTGGTTGAATTTGAACCTG | |
| | CATTTATTATGCTTCATCTAAATTGCATTCAAAGCTAACATT | |
| | AGAAAATTTCAACCTAAATTAAAATTCTTACATTTTCATTTA | |
| | AATGAATAAAACCCCCAACTTAAAACTGCGTAAACTAAGAGG | |
| | GTGATCTCAGGGGAGATGCTGTTGTGTTTCAAAGATAACCTC | |
| | AACGTGTTTAAAGTATGAGCTGAAAATCTTGGATGAGTTTCA | |
| | GAAGATAGAAAGAACCTAAAAGCCTAAAAGCCCAGAATCAAG | |
| | CTATGTTTTGTTGAGAGAAGCACACTGGCATTCTTGACCTGG | |
| | ACTCTTGGTCATGGTAGTATGAAGCTCAACCACAAGGCATGT | |
| | TGCTCAATGATGTCCTGGCCTCCTCATGTGGCCATCTCTGAT | |
| | ATAGCTATGTTATAGGATACCTTAGGGAATGTCAGTTCCTCT | |
| | GCCTGGCTGAAGAGGCATGATGTCTGAGTCAGTCCCATAGTG | |
| | TGAGAATCTCTTCTCCAAGATAAGATGACGTCAAGTATGGAA | |
| | CCCTTGGTACATGCCTGGCAGATGTTTGGTTTGTATTTCCAT | |
| | CCGGGAGATGCCACCCCTGGGCTTGTCCTGAAAACAACTAAG | |
| | TCCAAGGGGTTATTAGACTGCTTACAGAAGGCTTCATCTAGA | |
| | AAGTAAAAACAAAACTTTTAATGTATGAGCCATGCATAATCA | |
| | TTGCTAAAACATAAAAACAAAACAGAGAAAATCTAAAAGGAA | |
| | ATAAAAATTACACTATTCAAATATGACTACTTCTAAAATTTT | |
| | CATGTGTATTCTCCCAGAATTTTTTCTATATGTGATATGAAT | |
| | GTATTTTTTAGGCAAAATTGAGAGCACACTGTAAAGCTCTAT | |
| | GACACTGAACAAGTGACATGAATTTCCTGAGGTTTAGGACTC | |
| | TTTTCAACACAATAGGAGTGATGATACCATAAGGATTATTGC | |
| | TTCCTCTCAAAATGCTGCTGCCGTGCCGTAGGAGGCCCAAGG | |
| | CACATGCACAGGCCATGCATGAGTGCTCAAAGAATCGACCCT | |
| | GCTGAGCCCAGCCTTAAAATCATCCCAGCCCGGGCATCGGCC | |
| | ACATGAGTGAAAGACCCTGGGTGATTCCAGCCCCCTGACATT | |
| | TGAGTCACCCATAACTATTTGAGTCTTTCTAAATTAGACCCC | |
| | AGACACTGTGGAGCAGAGAAAAGCCATCCCCACTGTGCTCTG | |
| | TCCAATTTCTGACCCACAGAAGCAGCGTGCATAAGATGGTGA | |
| | TTGTTTTACTTTACATTTTGCATATGGTGTGGTTTGTTTTGC | |
| | AGCAACAGTGTCTATAACTGGACTGTTAACTTTGAGGAGCAC | |
| | TTTAGAGCAAACCCTTACCTGTGTGTGGCATCCCTTTCCATT | |
| | TCCTCAAGGAACTTGTTCTTCGGGTTGTTTTCTCTCTATCCT | |
| | GACTCTTCTGTCCCTCCTACTCCTTCCATTCAGCCTAAAAGT | |
| | GGAGTCAAGCCTTCTGCAAGGGGAAAATCCCTTCCCCTGTCT | |
| | CCATCATTTTGAACTACAATCTTTCCCTCACCAATTCCTCAT | |
| | CA | |
| SOX11 mRNA | GCAGAAGGAGCGCGCGCGCCTGGGAGAGCTCGGGGTCCGGCG | SEQ ID |
| sequence | CTTGCGGTAGGAGCCACGAGCCGGAGAGAGGGGTCCCGGGCT | NO: 39 |
| | GCCTCGACCGCCGTCGCCACCGCCTCTCCTGTCGCGACCGCA | |
| | GCTCCCACCGCGCCGGCGGCCGTCGTCGCCGAAGCCACCACA | |
| | GCCGCTGTGTGCAGCCTGGAAGGGGGGGCGGGGGGGAGGGGG | |
| | GGAGCCGCGAAAGCGGGGTGCCGAGGACTTTGCAACTTGCCC | |
| | AGGAAGGTGGAGGGGTGGGAGGGGGAGGGGGACCTCCGCACG | |
| | AGACCCAGCGGCCCGGGTTGGAGCGTCCAGCCCTGCAGCGGA | |
| | TCATGGTGCAGCAGGCGGAGAGCTTGGAAGCGGAGAGCAACC | |
| | TGCCCCGGGAGGCGCTGGACACGGAGGAGGGCGAATTCATGG | |
| | CTTGCAGCCCGGTGGCCCTGGACGAGAGCGACCCAGACTGGT | |
| | GCAAGACGGCGTCGGGCCACATCAAGCGGCCGATGAACGCGT | |
| | TCATGGTATGGTCCAAGATCGAACGCAGGAAGATCATGGAGC | |
| | AGTCTCCGGACATGCACAACGCCGAGATCTCCAAGAGGCTGG | |
| | GCAAGCGCTGGAAAATGCTGAAGGACAGCGAGAAGATCCCGT | |
| | TCATCCGGGAGGCGGAGCGGCTGCGGCTCAAGCACATGGCCG | |
| | ACTACCCCGACTACAAGTACCGGCCCCGGAAAAAGCCCAAAA | |
| | TGGACCCCTCGGCCAAGCCCAGCGCCAGCCAGAGCCCAGAGA | |
| | AGAGCGCGGCCGGCGGCGGCGGCGGGAGCGCGGGCGGAGGCG | |
| | CGGGCGGTGCCAAGACCTCCAAGGGCTCCAGCAAGAAATGCG | |
| | GCAAGCTCAAGGCCCCCGCGGCCGCGGGCGCCAAGGCGGGCG | |
| | CGGGCAAGGCGGCCCAGTCCGGGGACTACGGGGGCGCGGGCG | |
| | ACGACTACGTGCTGGGCAGCCTGCGCGTGAGCGGCTCGGGCG | |
| | GCGGCGGCGCGGGCAAGACGGTCAAGTGCGTGTTTCTGGATG | |
| | AGGACGACGACGACGACGACGACGACGACGAGCTGCAGCTGC | |
| | AGATCAAACAGGAGCCGGACGAGGAGGACGAGGAACCACCGC | |
| | ACCAGCAGCTCCTGCAGCCGCCGGGGCAGCAGCCGTCGCAGC | |
| | TGCTGAGACGCTACAACGTCGCCAAAGTGCCCGCCAGCCCTA | |
| | CGCTGAGCAGCTCGGCGGAGTCCCCCGAGGGAGCGAGCCTCT | |
| | ACGACGAGGTGCGGGCCGGCGCGACCTCGGGCGCCGGGGGCG | |
| | GCAGCCGCCTCTACTACAGCTTCAAGAACATCACCAAGCAGC | |
| | ACCCGCCGCCGCTCGCGCAGCCCGCGCTGTCGCCCGCGTCCT | |
| | CGCGCTCGGTGTCCACCTCCTCGTCCAGCAGCAGCGGCAGCA | |
| | GCAGCGGCAGCAGCGGCGAGGACGCCGACGACCTGATGTTCG | |
| | ACCTGAGCTTGAATTTCTCTCAAAGCGCGCACAGCGCCAGCG | |
| | AGCAGCAGCTGGGGGGCGGCGCGGCGGCCGGGAACCTGTCCC | |
| | TGTCGCTGGTGGATAAGGATTTGGATTCGTTCAGCGAGGGCA | |
| | GCCTGGGCTCCCACTTCGAGTTCCCCGACTACTGCACGCCGG | |
| | AGCTGAGCGAGATGATCGCGGGGGACTGGCTGGAGGCGAACT | |
| | TCTCCGACCTGGTGTTCACATATTGAAAGGCGCCCGCTGCTC | |
| | GCTCTTTCTCTCGGAGGGTGCAGAGCTGGGTTCCTTGGGAGG | |
| | AAGTTGTAGTGGTGATGATGATGATGATAATGATGATGATGA | |
| | TGGTGGTGTTGATGGTGGCGGTGGTAGGGTGGAGGGGAGAGA | |
| | AGAAGATGCTGATGATATTGATAAGATGTCGTGACGCAAAGA | |
| | AATTGGAAAACATGATGAAAATTTTGGTGGAGTTAAAGTGAA | |
| | ATGAGTAGTTTTTAAACATTTTTCCTGTCCTTTTTTTGTCCC | |
| | CCCTCCCTTCCTTTATCGTGTCTCAAGGTAGTTGCATACCTA | |
| | GTCTGGAGTTGTGATTATTTTCCCAAAAAATGTGTTTTTGTA | |
| | ATTACTATTTCTTTTTCCTGAAATTCGTGATTGCAACAAAGG | |
| | CAGAGGGGGCGGGGCGGGGGAGGGGAGGTAGGACCCGCTCCG | |
| | GAAGGCGCTGTTTGAAGCTTGTCGGTCTTTGAAGTCTGGAAG | |
| | ACGTCTGCAGAGGACCCTTTTGGCAGCACAACTGTTACTCTA | |
| | GGGAGTTGGTGGAGATATTTTTTTTTCTTAAGAGAACTTAAA | |
| | GAACTGGTGATTTTTTTTTAACAAAAAAAGGGACCATTGCAA | |
| | CTTTTGTTAATTTAATTTTTTTTTTTTTTTTTTTTTTTTTTT | |
| | TTTTTGGAGGGAGAAAACTGATGTCTTCTATGCATCCGATTC | |
| | TTAACAAAACTGCAGGGAGCTTGAAAAAATGCAGACTGTACA | |
| | AACGCTTACAAAAAAAAAAACTGTGAACTGACTTAAGATCAG | |
| | AGTTTACTTTTCAGATCAAATTGTTTATGGTTTTACAAATGT | |
| | GATTTCTACTTGCCAACTTTTTTTTTGTAACTTGTTCCCTTA | |
| | TACCTCCTTGATTGAATACCAGACAGCCTAGACCTCAGTACA | |
| | AAAGGTATTGAAACATTTTTGATACATAACAGACCTCAGTCT | |
| | TTTTTAAAAATTAATATATTTTCAGGCGTATTTTTGTACAGT | |
| | GAAAAGGGAACATTCTTGCTGTGTTTTTTCAGTAAGACTTTC | |
| | AGGCACTTCTTCCCTTTTGATTTCTTTTTTTTCCTCTGTTTT | |
| | TTAGCATGCAAGTATGTTGGTACGTTATGTCCTGGTTTAAAA | |
| | AGGATTAAAATTTTAAAATAATCCTTGCATCTAAAGGCCTTG | |
| | TGGTTTAAAAAAAAAAAGCAAACTTTTTTTTGTACAGCTATA | |
| | GTAGAGATTTGTTCAATATTTGTAGGTAAAGATTTATTGAAA | |
| | ATGGTGATATAGACCTCAGAGCTGTTATCTTAGTTTAAAGAT | |
| | TGTATATGTACTGTACTATAGTAGGACTTTATGTATCTCATA | |
| | CGCTGTGATGTGGATGGGGCCCCAGATGGAAGGTTTGAAACT | |
| | GGATTCTCGATTTTTAGCAAAAAAGAAAAAAAAAAGGCACAT | |
| | AGTTTAAAAAGTTTCTCATTTTGTGCAATATAATCTAAATAA | |
| | AGTACAGACCATCTGCATATTTTGTAGCAAATGGTGGCAAAG | |
| | CAGACTCAATGCACTGTCGACATCATTGCCTGTTTTTTTTTT | |
| | TTTTTTTTTGTGCTGGAAGTCTGTATCTTGACAATTTTAATA | |
| | AATCAGCTGGAACTGATAGAAACTCGCATCGCCAATAGTCTC | |
| | TATGGAAGTCAAACTGGAGGTCCTGTTGTCGCAGAGCATTCG | |
| | GTGGTGAGGCTGTTGTGTGCGCGGATGAGGGGAGGTGGCAGG | |
| | AGAGAATTCTACATTTAGGGGGTTAGGCTGAAAAGTGTTCAA | |
| | TTAGCAGGCTGATTTCTTTTTCCTCTTCCGCTAGTIGTGAAA | |
| | GACAGGGGAAGGGTGTTCTTTCTCTCTGCCCTCCCTTTCCAT | |
| | CTCCAGCTCCCCATTTCCTTTCTCACCTCCTCCTCCACTCCC | |
| | TGCCTCTTCTCCCCACCCATCCTGGCGGGCGGGCTGCGCGGA | |
| | GGCTCGGGAGCTGGCCGGGGAGGGGCGGATGGAGGGGCCTGG | |
| | GTTGCCAGCTCCCTTGGTCGGGGTCCTGCTCGCTGGGGCTTG | |
| | TGTGTTCTCTGCGGCGGGCCGCGTCCCCGCTGAGCCTCGCGG | |
| | TGACAGCCGCCTTTGGCAGCGAGCGCTCGGGGCACTTCTATC | |
| | CCCGCCTCTCAAAGGGTGGGGACAGCCGTTTCCAGATTTGAA | |
| | TTTTTTCTGTTCTTTATTTTTAACGCTGCATCTTCGCGTGTG | |
| | CTCAGAGGTGGTTGTTGGCGGAGAACGCCGCCGCAGTGTTTG | |
| | ACCTCTAGCGGTGAAGGGGGAAGGGGAAGAGGAAAGGAGAGA | |
| | AGTGGTCGGTGTCTGTTTCCTTCTGTCCCCCGGGGCCGTGGA | |
| | GCTGTCGGAGGGAAGGAGGACGGTGCGGGGCCGCAGGGGGCG | |
| | CGGGGCGCGGCGGGACCCAGGCTACGAGCGGGAGGGAGGCGG | |
| | GAGTCGGGGGAAGACGCGGCAGGCCGGCCGAGGGCACCCCGA | |
| | GGAACATGGCATGGCCTCTGTGCGATCCGAGTCGCGGTCTCC | |
| | GGGGTGCCTGGGAGGGCCGAACCACTGGTGAGGGCGTGGGGA | |
| | GCAGGGGGTGGCAGAGGGCACCCGGGCGGTAGTCCGGGACGC | |
| | GCAAGGCAGAGCCCTGACGCTCCGGGTCCCCGTGCCTGGCTC | |
| | TTCTTGCCTCGCCACCGCGTGCTCCTGGGCGCGCCCCGCCGC | |
| | GGGCCCTTGAGGCGCGCGGAGACACCAGCGCTGGCTTCCCGG | |
| | GCCCGCGGGCCGGGGAGGGAAGCCTCGGGGCTGCGGGGTGAG | |
| | AGGAAGAAAGCAAACCCGGGGAGCAGGCGGCTGCCGCACCCG | |
| | CGCACCCCGGGCCCTCACCACGCCCTCCCCGCGCGCCGGCTC | |
| | AGGGGCTGCCCCGGAATCAGCTCCCCGGGGCCGCCGCAACGA | |
| | AGGTGGATCCGCATCTTGATTGTTCTCCGGGAGCCTCCTGGG | |
| | GGCTCCGGCGGCGGCGCGGGCGCGACCCATCCCGCTGGCGCT | |
| | CCCCGCTCGCTGAACCCCGTTTGCCTGTCCACACCCCCTCGC | |
| | TCCCCACCATTTTTCCTGACCGGCCTGTGTCCCCGAGCCCTC | |
| | GCGGCAGGCCCGAGCAGGCGATCGCGGCCGGGCACGCGCGCC | |
| | CCGGGCTCCCGCCCCCCTTCCGAGCATCCGCCGCCTCTTTTC | |
| | TGCTGGGTCTGGGAGGAGGGAGGCTGGGAGGCCGCTCGGGGC | |
| | CCAGCGTGCCAGCCCCGGAGTTCAGCCTCCCGAGCTGCGGCG | |
| | CCCGCAGCGGAGGAGGTTTTCAGTGGCTGATTGAAACTCACT | |
| | GCAAAATCACCACGACTCTTTCACCTACTGAGATGATTGACC | |
| | GAGGTTTGGCCTTCCATTTTTACTGAGATTTGGCGAGACCGA | |
| | ATGGAAGCGTCCGCACAGTAACTGCAGCTGCTAGGCCAGAGG | |
| | GGCCCCGGCGCCCTTCCCGCCTCCCCTCCCGCTTGCTTTTGC | |
| | CTTACTCGATCTTACCACCACCCCTCCCCCGGCCCCCCGACT | |
| | GAGAACTCGGGCCTCTCACCCGCCCCCCAGCCTCCCGCTCTG | |
| | GGCGAGCCTCCTCCCCAGCCCCCACCCCTGGGATGCGAAGCC | |
| | AGCAAGCTTTTGCTGCAGATGGACAGGTTTCTTTTCTGTGGC | |
| | TTTTTCCTTTCGATAAACCATCAGATTTCAGTAGTACATTTG | |
| | GGAAAAGAAGGGGCTGATGGCGTTAACCAGGTTCTCAATATA | |
| | GAACTGGATTTCTGGAGTTGTTTACCTTACCCCACACCCCCT | |
| | CAACATGTAGACTAATGCAGCCATTGGTGGTACATTTATTTT | |
| | AGCCACGGATAATTGAACCAGCGGTTTACAATTGACACGTGC | |
| | TCCGTGCTGGTGATTTTATGTGGCAGCCCTCTGCTGCAGTTC | |
| | CGAAACTTGTTGGCAACGTAAACCCATTGATAGGCTGATCTA | |
| | TGTATTTTGAAAGCCTGAAAACTTGGCATGTCTTTTCTGTTT | |
| | TAATCATAGATGAATCTTGGACATTTTCTGTGGTGAGGTGGA | |
| | AACTTTAAGTAAATTAGTAAAGTAATAATTTGGCTTCAGAAT | |
| | GGGAAGAGATAGTCAAGATTTTTTTTTTTTAAAGCCATGTGG | |
| | CCTAACTTGATACAAAAATAAAAGTAATTGTTTGGCAATCTA | |
| | AATTTAAAACCTGTTAGAACTCAGGACAGGCGCTTCAATGCG | |
| | CTTTTTAACAATATTTAAGGCTGTTTTGATGAGTGCGTTGTG | |
| | AGAATCATCTTAATGAATTCTTTATTGAGTGTCTAAAACATA | |
| | GTATAATACACATGGTATTCTTGCCACTGGATAGTCTTCAAT | |
| | AAAAGTTTAATTGATTTTTTTTTGTTGGTCTCTTAAGTAAGT | |
| | CTTATTTTTAACTAAGCATTGACAGAATATCTTAAAATGGTA | |
| | ACCTGGGGGTGGCGGGTGGGTGCTGTGTGCACGGCAGCCTAG | |
| | CCAGTGGGGATCCTGCTGTTTATTATAAGTAGTTCACAGACT | |
| | CTGATGGCATTTTGGTAAGCTTTCCATCTTTAAGAAATTGAA | |
| | CCAGCATTCTCTTATTAATTCTTTAAACTGTGGAAGTAATTT | |
| | CCAGTTCTTACACTCTGATACGCATCCCTTTTATTTAAAAAA | |
| | AAAAAAAATGCTAATAAAAGGCAGTGTACTTAAACTGTGCTT | |
| | TGCAAATATTGTGTATGTTATGAATGACTACAGACACTGGGC | |
| | AAATTATTTGTAGAATGATTATCCTTTAGCTAGAGAAAGAAA | |
| | TCATTACAACTCTTTTGGGCAGAGATGTTTCTTTTTAATGTT | |
| | AATCAAGGGGAAGTGATTTAAATATGCATAAATGTAGCAGTC | |
| | AGGGTGATTTAGTTGCTTTTTTCATGAAAGAAAAAGACTCAA | |
| | AAGACAAGACTTATTTTTCTCTTCTGGGACTTGAAATCATAA | |
| | TCATCTGATATTAGTACAGTACAAGAAATTTACATTTGTTTT | |
| | TTACTTCAGAATTTAAGTGACTTTTGCCCAAGGAATTTGAGA | |
| | AATAAGGCAAATAAGTTGCTCTATTTTAAAGTAGTCATTCAA | |
| | TATAAATATATTATATCAATCTTAACTTTTTTATTCTCTGAT | |
| | ATGATTAATAATATGTATATTCTTACTTTTCTTCTAATGGGC | |
| | ATATGTATCCTTGTGGACACTTTGAGAGAGGTTTTCTTGGAC | |
| | TCTCCCATTTATAGAATCTTTATACTCTTTTACTGTGTGGTT | |
| | CCCTGCTTTTAACAGATTTCTGAGGCAAATATATTTGTGCTT | |
| | TTTTCTTATGTAGGAAGACCAGCGAAAATAGTTTACTGAGTT | |
| | GTCAATTTTATCAGTAGATAAGAAACTTTCTTTATTACAGTT | |
| | TCAGGGAAGATTTTTTCAGGATATTTCTCAGTTATTCTAAGG | |
| | GCCAAATTTTGTAAAATTTCCATTAGGAATGTCAGTTTCAAA | |
| | TACCCTTTGTATAGCCTAAGCCTGTGAGGATAACAAGAATGA | |
| | GCCTTACCTATCCTAACACAGGGATTTACAAGTTCCCAAAGT | |
| | AACCGTCTCCATGTAACTCTTGACATACTTTTCTGAGATTTG | |
| | GCTTATTTTTATTATTGGTTATTTCTCACTGTTCATTCTATT | |
| | TGATTTATTCTACAACATCCCCTTTTATTTAATGATCTGGAA | |
| | AATTCTGCTCTTTGATAACAACTCAGGATTTTTTTGTTCAGT | |
| | TTTGGTTTTTGCCCCTTCCTGTGGAGCCTACATTTTCAACCA | |
| | CAATAAAGATGAAACAAAATTTATGAAACTGAGCTCTCTTCC | |
| | ATTTTACTTACTGCTGGCTTTTTTTTTTTTTTTTTTCCTTGA | |
| | TTCCTACCATACCTTCGTTTTTTTCATTGTACTTTTTTAACA | |
| | CTACCTATATCCATTAGCTGCCTAATTAGTTTTATCTGTTCC | |
| | ATGTGGATGCAGTGAGTTTATAAGAGAATTTCACAAACAAGT | |
| | AGTTTTTTAGTGAACTTAAAATAAACAGAATTTTAAAGGAGA | |
| | CCTATTTTTATACTCAATAAAAGCACAAAAGTGCAGAAAGTA | |
| | TAAAACGGCTTACAAAGGGAGACACAAGCTCATAATGTTCCA | |
| | TGTATAAAAGTAATAACTTTATTGGGTAGAGATATTCTTACA | |
| | AGATCTAGCACCTCTGCCAGTGCACAGATAGGACTGTTTTAA | |
| | ATGATTTGGGAACTTTTGGTTGCCTGCAGTTGTGAACAGAGA | |
| | ACTTCTCTACAGAGAAACAAACCACTAAAAGCAATATGACCG | |
| | AGTTGAGATGTGGTTTCCAATGAGCAATTGGTGAATTTAAGC | |
| | AACCTGGATGTGCATATGTGGAGGCTCCCGTCTCACTGTTTG | |
| | ATCAAACTTCTTTTATGTAGTCACGTAGACTTGATTTTTTCT | |
| | GCTGTGAAAATGAAAAAATAAAGCAATATGACAAAAAGTTTA | |
| | AAAATGCATAAAAAATAGGATTTCCTCTAGGCTCCTCGAAGA | |
| | GATTTTTTTAATATGATGCTTGTCTTACTTTCTTAGACACGT | |
| | TACATTTCCCCTTCCAAAAAAAAAAAAAAGGACAACTGGAAG | |
| | TAATTTATCATATAAAGAATTTTGATCAAATAGATATTGACA | |
| | AAGGGCCCTCTGTCACATTTTTCTTCATCCAGCTTTTGTTCA | |
| | AAAACAGTATGCCTCCTCCCTTGAATCACATAGGGAGAAACG | |
| | TTATACTCCATTCTCATTAATTTCCCATTTTGTCTACTTTTA | |
| | CTCTTGTACATATGTTGTGGGTTTAAGAGTCTTTTGCATTTG | |
| | TTCTGTGACACCTTTTTTTGAATTGACTGTTTTAAAACGGAG | |
| | GCCTATTTTTTCCGGTTTGGGACTCCTAGTGGTTATGGCATC | |
| | CCATAATGCTTCGTGACGGCCACCAGGACAGAACCACCTGAT | |
| | GTTTTAGAGCAGTTTTCAGCATGACACTGTTAACAAGTGTGT | |
| | ATTTTCCAAGGCCACATGAAACTTACTTTCTTAGCCACTCCA | |
| | GGTTTGGGAGCAGAAAAGCTGAAAAACCCTTTTGTGTAGAAG | |
| | TCTGAGTGGTTTGTGGGGGGGACCTTTTTTAGAGTTTGCATG | |
| | CCAGCGCACGGCCTATTGCTGTGAAACAGAGAGAAGGTAAAG | |
| | CTACCTGAGGCAGTGCGCTGGAGGATGAAGTGTTTGATAGCA | |
| | CTAGGGGGGAAAGAAAATGCATGGCAAAGTTTCGTCTTCTCG | |
| | TAGACTATCTAGCATGCAGAGTGTAGTGTGTTGAAACGGTGT | |
| | ATGACATTGCTGTATCAAAGTTGTAAAATTAAGCATTATTTA | |
| | TTGAAAACTATGTATTTTTTTGTAAAAACCTGATCACATAGA | |
| | GAATATCAGTGGCTTGTGCTTGTGCTTCGATCTAACCAGCTT | |
| | CTTGACCCACCCCCCCTTGGTATGCAGTGTTAATGCTCAGGG | |
| | TTGAAAATAGTACACTCCAATGTCTCTTTTGCAAGAGTTTTT | |
| | CACAGAGGATTACATTTGTTCAAAAGACTCTAATAAAATTGT | |
| | GTGATCAATCTTCA | |
| HNRNPH1 | CATTTCGTCTTAGCCACGCAGAAGTCGCGTGTCTAGGTGAGT | SEQ ID |
| mRNA | CGCGGTGGGTCCTCGCTTGCAGTTCAGCGACCACGTTTGTTT | NO: 40 |
| sequence | CGACGCCGGACCGCGTAAGAGACGATGATGTTGGGCACGGAA | |
| | GGTGGAGAGGGATTCGTGGTGAAGGTCCGGGGCTTGCCCTGG | |
| | TCTTGCTCGGCCGATGAAGTGCAGAGGTTTTTTTCTGACTGC | |
| | AAAATTCAAAATGGGGCTCAAGGTATTCGTTTCATCTACACC | |
| | AGAGAAGGCAGACCAAGTGGCGAGGCTTTTGTTGAACTTGAA | |
| | TCAGAAGATGAAGTCAAATTGGCCCTGAAAAAAGACAGAGAA | |
| | ACTATGGGACACAGATATGTTGAAGTATTCAAGTCAAACAAC | |
| | GTTGAAATGGATTGGGTGTTGAAGCATACTGGTCCAAATAGT | |
| | CCTGACACGGCCAATGATGGCTTTGTACGGCTTAGAGGACTT | |
| | CCCTTTGGATGTAGCAAGGAAGAAATTGTTCAGTTCTTCTCA | |
| | GGGTTGGAAATCGTGCCAAATGGGATAACATTGCCGGTGGAC | |
| | TTCCAGGGGAGGAGTACGGGGGAGGCCTTCGTGCAGTTTGCT | |
| | TCACAGGAAATAGCTGAAAAGGCTCTAAAGAAACACAAGGAA | |
| | AGAATAGGGCACAGGTATATTGAAATCTTTAAGAGCAGTAGA | |
| | GCTGAAGTTAGAACTCATTATGATCCACCACGAAAGCTTATG | |
| | GCCATGCAGCGGCCAGGTCCTTATGACAGACCTGGGGCTGGT | |
| | AGAGGGTATAACAGCATTGGCAGAGGAGCTGGCTTTGAGAGG | |
| | ATGAGGCGTGGTGCTTATGGTGGAGGCTATGGAGGCTATGAT | |
| | GATTACAATGGCTATAATGATGGCTATGGATTTGGGTCAGAT | |
| | AGATTTGGAAGAGACCTCAATTACTGTTTTTCAGGAATGTCT | |
| | GATCACAGATACGGGGATGGTGGCTCTACTTTCCAGAGCACA | |
| | ACAGGACACTGTGTACACATGCGGGGATTACCTTACAGAGCT | |
| | ACTGAGAATGACATTTATAATTTTTTTTCACCGCTCAACCCT | |
| | GTGAGAGTACACATTGAAATTGGTCCTGATGGCAGAGTAACT | |
| | GGTGAAGCAGATGTCGAGTTCGCAACTCATGAAGATGCTGTG | |
| | GCAGCTATGTCAAAAGACAAAGCAAATATGCAACACAGATAT | |
| | GTAGAACTCTTCTTGAATTCTACAGCAGGAGCAAGCGGTGGT | |
| | GCTTACGAACACAGATATGTAGAACTCTTCTTGAATTCTACA | |
| | GCAGGAGCAAGCGGTGGTGCTTATGGTAGCCAAATGATGGGA | |
| | GGCATGGGCTTGTCAAACCAGTCCAGCTACGGGGGCCCAGCC | |
| | AGCCAGCAGCTGAGTGGGGGTTACGGAGGCGGCTACGGTGGC | |
| | CAGAGCAGCATGAGTGGATACGACCAAGTTTTACAGGAAAAC | |
| | TCCAGTGATTTTCAATCAAACATTGCATAGGTAACCAAGGAG | |
| | CAGTGAACAGCAGCTACTACAGTAGTGGAAGCCGTGCATCTA | |
| | TGGGCGTGAACGGAATGGGAGGGTTGTCTAGCATGTCCAGTA | |
| | TGAGTGGTGGATGGGGAATGTAATTGATCGATCCTGATCACT | |
| | GACTCTTGGTCAACCTTTTTTTTTTTTTTTTTTTTTTCTTTA | |
| | AGAAAACTTCAGTTTAACAGTTTCTGCAATACAAGCTTGTGA | |
| | TTTATGCTTACTCTAAGTGGAAATCAGGATTGTTATGAAGAC | |
| | TTAAGGCCCAGTATTTTTGAATACAATACTCATCTAGGATGT | |
| | AACAGTGAAGCTGAGTAAACTATAACTGTTAAACTTAAGTTC | |
| | CAGCTTTTCTCAAGTTAGTTATAGGATGTACTTAAGCAGTAA | |
| | GCGTATTTAGGTAAAAGCAGTTGAATTATGTTAAATGTTGCC | |
| | CTTTGCCACGTTAAATTGAACACTGTTTTGGATGCATGTTGA | |
| | AAGACATGCTTTTATTTTTTTGTAAAACAATATAGGAGCTGT | |
| | GTCTACTATTAAAAGTGAAACATTTTGGCATGTTTGTTAATT | |
| | CTAGTTTCATTTAATAACCTGTAAGGCACGTAAGTTTAAGCT | |
| | TTTTTTTTTTTTAAGTTAATGGGAAAAATTTGAGACGCAATA | |
| | CCAATACTTAGGATTTTGGTCTTGGTGTTTGTATGAAATTCT | |
| | GAGGCCTTGATTTAAATCTTTCATTGTATTGTGATTTCCTTT | |
| | TAGGTATATTGCGCTAAGTGAAACTTGTCAAATAAATCCTCC | |
| | TTTTAAAAACTGCA | |

ChIP-qPCR assays were performed as previously described. Briefly, cells (2× 106) were transduced with lentivirus expressing non-targeting shRNA control, shHNRHNP1 or shSOX11 for 60 h (selected with puromycin) and then fixed with 1% formaldehyde for 10 min at room temperature and quenched with 0.2 M glycine. Sonicated chromatin was prepared in buffer (10 mM Tris-HCl pH 8.0, 1 mM EDTA, 0.5 mM EGTA and protease inhibitor cocktail) and then incubated with 4 μg of H3k27ac antibody (Abcam, ab4729) overnight at 4° C. Magnetic protein A/G beads were incubated to each ChIP reaction for 1 h at 4° C. ChIP DNAs were eluted into 200 μl of elution buffer at 65° C. for 20 min and extracted with phenol/chloroform. Purified DNAs were subjected to RT-PCR assay for quantifying H3K27ac occupancy on the enhancers. Sequences of ChIP-qPCR primers are listed in Table 1.

### Cut&Run-Seq and Data Processing

Cut&Run-seq was performed as previously described. Briefly, 200,000 cells were gathered, washed twice and captured by the addition of 10 μl of pre-activated concanavalin A coated magnetic beads (Bangs Laboratories-BP531). Cells were then resuspended in 100 μl cold Antibody Buffer and 1 μl antibody (H3K4me3, EpiCypher no. 13-0041; H3K27Acs, Active Motif no. 39133; SOX11, Sigma catalogue no. HPA000536 or HNRNPH1 Abcam catalogue no. ab154894) was added for incubating on a nutator overnight at 4° C. Cells were washed twice in 1 ml of Digitonin Buffer (20 mM HEPES-KOH PH 7.5; 150 mM NaCl; 0.5 mM Spermidine; 1× Roche cOmplete™; 0.05% digitonin), and then resuspended with CUTANA pAG-MNase in 50 μl of Digitonin buffer. After washing twice, samples were quickly mixed with 100 mM CaCl₂) to a final concentration of 2 mM and incubated at 4° C. for 30 min, then the reaction was quenched by Stop Buffer. The cleaved fragments were released by incubating the tube for 30 min at 37° C. and then purified by Qiagen MinElute PCR Purification Kit. Libraries were prepared with NEBNext Ultra II DNA Library Prep Kit for Illumina (E7645) and sequenced by NovaSeq PE150.

Cut&Run-seq reads were aligned to the reference human genome v.hg38 with the program BOWTIE v.2.3.4.1. Aligned reads were stripped of duplicate reads with the program sambamba v.0.6.8. Peaks were called using the program MACS v.2.1.2 with the narrow and broad peaks mode for Cut&Run-seq. Motif enrichment analysis was performed for both HNRNPH1 and SOX11-bound sites using HOMER find MotifsGenome function with—size 1,000—mask settings. Transcription factors with high-expression level in G3 MB cell lines and significant H3K27Ac enrichment in G3 MB-specific enhancers were identified as G3 MB-specific active motifs.

### Statistics and Reproducibility

All analyses in this research were performed using Microsoft Excel, GraphPad Prism 6.00 (https://www.graphpad.com) or RStudio (https://www.rstudio.com/and R v.4.0.3, R Development Core Team, 2016). The ‘cor’ function in R was used to calculate the Pearson correlation coefficient. Statistical significance was determined using two-tailed Student's t-tests as indicated. A one-way analysis of variance test was performed by multiple comparisons following Tukey's ranking tests when comparing multiple groups. Data are shown as mean±s.e.m. Values of P<0.05 denoted a statistically significant difference. Quantifications were performed from at least three experimental groups in a blinded fashion. The n value was defined as the number of experiments that were repeated independently with similar results. No statistical methods were used to predetermine sample sizes, but our sample sizes are similar to those generally used in the field. No randomization was used to collect all the data, but data were quantified with blinding.

### ADDITIONAL REFERENCES

1. Bandopadhayay, P. et al. BET bromodomain inhibition of MYC-amplified medulloblastoma. Clin. Cancer Res. 20, 912-925 (2014).
2. Zhang, L. et al. Single-cell transcriptomics in medulloblastoma reveals tumor-initiating progenitors and oncogenic cascades during tumorigenesis and relapse. Cancer Cell 36, 302-318 e307 (2019).
3. Luo, Z. L. et al. A splicing variant of Merlin promotes metastasis in hepatocellular carcinoma. Nat. Commun. 6, 8457 (2015).
4. Stuart, T. et al. Comprehensive integration of single-cell data. Cell 177, 1888-1902 e1821 (2019).
5. Butler, A., Hoffman, P., Smibert, P., Papalexi, E. & Satija, R. Integrating single-cell transcriptomic data across different conditions, technologies, and species. Nat. Biotechnol. 36, 411-420 (2018).
6. Trapnell, C. et al. The dynamics and regulators of cell fate decisions are revealed by pseudotemporal ordering of single cells. Nat. Biotechnol. 32, 381-386 (2014).
7. Wang, Y. H. et al. A CIBERSORTx-based immune cell scoring system could independently predict the prognosis of patients with myelodysplastic syndromes. Blood Adv. 5, 4535-4548 (2021).
8. Whitfield, M. L. et al. Identification of genes periodically expressed in the human cell cycle and their expression in tumors. Mol. Biol. Cell 13, 1977-2000 (2002).
9. Tirosh, I. et al. Single-cell RNA-seq supports a developmental hierarchy in human oligodendroglioma. Nature 539, 309-313 (2016).
10. Corces, M. R. et al. The chromatin accessibility landscape of primary human cancers. Science 362, eaav1898 (2018).
11. Wang, J. et al. Epigenomic landscape and 3D genome structure in pediatric high-grade glioma. Sci. Adv. 7, eabg4126 (2021).
12. Wu, L. M. N. et al. Programming of Schwann cells by Lats1/2-TAZ/YAP signaling drives malignant peripheral nerve sheath tumorigenesis. Cancer Cell 33, 292-308.e297 (2018).

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular aspects of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method of treating a medulloblastoma (MB) tumor in an individual in need thereof, comprising administering to the individual a composition comprising one or more of:
a. a nucleic acid having specific binding to a SOX11 mRNA;
b. a nucleic acid having specific binding to an HNRNPH1 mRNA; and
c. a nucleic acid having specific binding to a MYC enhancer region.

2. The method of claim 1, wherein the nucleic acid having specific binding to a MYC enhancer region impedes binding of one or both of a SOX11 protein and a HNRNPH1 protein to the MYC enhancer region.

3. The method of claim 1, wherein the nucleic acid is a ribonucleic acid (RNA) or a deoxyribonucleic acid (DNA).

4. The method of claim 1, wherein the nucleic acid is a small interfering ribonucleic acid (siRNA).

5. The method of claim 1, wherein the nucleic acid is a single-guide ribonucleic acid (sgRNA).

6. The method of claim 5, wherein the sgRNA is complementary to the MYC enhancer region.

7. The method of claim 6, the MYC enhancer region comprising a sequence having at least 90% homology to any one of SEQ ID NO: 33, SEQ ID NO: 34, SEQ ID NO: 35, SEQ ID NO: 36, SEQ ID NO: 37, SEQ ID NO: 38, and combinations thereof.

8. The method of claim 5, wherein the sgRNA corresponds to one or more of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

9. The method of claim 5, wherein the sgRNA is delivered to a cell of the individual via a CRISPR, wherein the delivery removes a SOX11 and/or HNRNPH1 binding site.

10. The method of claim 5, wherein the sgRNA is delivered to a cell via CRISPRi, wherein the delivery interferes with a SOX11 and/or HNRNPH1 binding site.

11. The method of claim 1, wherein the individual is a pediatric individual.

12. The method of claim 1, wherein the MB is G3-MB.

13. The method of claim 1, wherein the MB is G4-MB.

14. The method of claim 1, wherein the administration slows, reverses, or stops MB tumor growth.

15. A composition comprising one or more nucleic acid of claim 1.

16. The composition of claim 15, further comprising a pharmacologically acceptable excipient.

17. A viral vector expressing an single-guide ribonucleic acid (sgRNA).

18. The viral vector of claim 17, the viral vector being a lentiviral vector.

19. A method for distinguishing G3 MB and G4 MB, comprising detecting expression of one or more of HNRNPH1, PPP1R14A, SOX11, and combinations thereof.

20. The method of claim 19, comprising detecting one or both of HNRNPH1 and PPP1R14A, wherein a higher level of one or both of HNRNPH1 and PPP1R14A, as compared to a control value, is indicative of a diagnosis of G3 MB.


SEQUENCE LISTING (40 sequences)

SEQ ID NO: 1 - DNA, 62 bp, synthetic construct
  source 1..62: mol_type = other DNA
gatccgcctctactacagcttcaagattcaagagatcttgaagctgtagtagaggctttttg

SEQ ID NO: 2 - DNA, 62 bp, synthetic construct
  source 1..62: mol_type = other DNA
aattcaaaaagcctctactacagcttcaagatctcttgaatcttgaagctgtagtagaggcg

SEQ ID NO: 3 - DNA, 62 bp, synthetic construct
  source 1..62: mol_type = other DNA
gatccgcaggaagatcatggagcagtttcaagagaactgctccatgatcttcctgctttttg

SEQ ID NO: 4 - DNA, 62 bp, synthetic construct
  source 1..62: mol_type = other DNA
aattcaaaaagcaggaagatcatggagcagttctcttgaaactgctccatgatcttcctgcg

SEQ ID NO: 5 - DNA, 62 bp, synthetic construct
  source 1..62: mol_type = other DNA
gatccgggacacagatatgttgaagtttcaagagaacttcaacatatctgtgtccctttttg

SEQ ID NO: 6 - DNA, 62 bp, synthetic construct
  source 1..62: mol_type = other DNA
aattcaaaaagggacacagatatgttgaagttctcttgaaacttcaacatatctgtgtcccg

SEQ ID NO: 7 - DNA, 62 bp, synthetic construct
  source 1..62: mol_type = other DNA
gatccgggctcaaggtattcgtttcattcaagagatgaaacgaataccttgagccctttttg

SEQ ID NO: 8 - DNA, 62 bp, synthetic construct
  source 1..62: mol_type = other DNA
aattcaaaaagggctcaaggtattcgtttcatctcttgaatgaaacgaataccttgagcccg

SEQ ID NO: 9 - DNA, 25 bp, Homo sapiens
  source 1..25: mol_type = other DNA
caccgtaaatcaagcagatggtcac

SEQ ID NO: 10 - DNA, 25 bp, Homo sapiens
  source 1..25: mol_type = other DNA
aaacgtgaccatctgcttgatttac

SEQ ID NO: 11 - DNA, 25 bp, Homo sapiens
  source 1..25: mol_type = other DNA
caccgggaaggatgcggacagccag

SEQ ID NO: 12 - DNA, 25 bp, Homo sapiens
  source 1..25: mol_type = other DNA
aaacctggctgtccgcatccttccc

SEQ ID NO: 13 - DNA, 25 bp, Homo sapiens
  source 1..25: mol_type = other DNA
caccggactatgtgaccatatgcca

SEQ ID NO: 14 - DNA, 25 bp, Homo sapiens
  source 1..25: mol_type = other DNA
aaactggcatatggtcacatagtcc

SEQ ID NO: 15 - DNA, 25 bp, Homo sapiens
  source 1..25: mol_type = other DNA
caccgcaggcaacatggcttgctaa

SEQ ID NO: 16 - DNA, 25 bp, Homo sapiens
  source 1..25: mol_type = other DNA
aaacttagcaagccatgttgcctgc

SEQ ID NO: 17 - DNA, 25 bp, Homo sapiens
  source 1..25: mol_type = other DNA
caccgcttagcagctccatgatctg

SEQ ID NO: 18 - DNA, 25 bp, Homo sapiens
  source 1..25: mol_type = other DNA
aaaccagatcatggagctgctaagc

SEQ ID NO: 19 - DNA, 25 bp, Homo sapiens
  source 1..25: mol_type = other DNA
caccggctacttgggagactgagac

SEQ ID NO: 20 - DNA, 25 bp, Homo sapiens
  source 1..25: mol_type = other DNA
aaacgtctcagtctcccaagtagcc

SEQ ID NO: 21 - DNA, 22 bp, synthetic construct
  source 1..22: mol_type = other DNA
gcaaatgtagagtacaggagag

SEQ ID NO: 22 - DNA, 20 bp, synthetic construct
  source 1..20: mol_type = other DNA
gtcttccaatttccaggcac

SEQ ID NO: 23 - DNA, 19 bp, synthetic construct
  source 1..19: mol_type = other DNA
gcatgtgtcatttccccag

SEQ ID NO: 24 - DNA, 19 bp, synthetic construct
  source 1..19: mol_type = other DNA
tccgcatccttcccttttc

SEQ ID NO: 25 - DNA, 20 bp, synthetic construct
  source 1..20: mol_type = other DNA
cccttctttcctccactctc

SEQ ID NO: 26 - DNA, 19 bp, synthetic construct
  source 1..19: mol_type = other DNA
acaaaccgcatccttgtcc

SEQ ID NO: 27 - DNA, 21 bp, synthetic construct
  source 1..21: mol_type = other DNA
cccagtaagaaccatccgaag

SEQ ID NO: 28 - DNA, 20 bp, synthetic construct
  source 1..20: mol_type = other DNA
tgaaggaagcaaaagccaac

SEQ ID NO: 29 - DNA, 21 bp, synthetic construct
  source 1..21: mol_type = other DNA
tccctaaaaatccccctgttc

SEQ ID NO: 30 - DNA, 21 bp, synthetic construct
  source 1..21: mol_type = other DNA
aacactgtccatttccaactc

SEQ ID NO: 31 - DNA, 21 bp, synthetic construct
  source 1..21: mol_type = other DNA
cctggtgctccatgaggagac

SEQ ID NO: 32 - DNA, 22 bp, synthetic construct
  source 1..22: mol_type = other DNA
cagactctgaccttttgccagg

SEQ ID NO: 33 - DNA, 5000 bp, Homo sapiens
  source 1..5000: mol_type = other DNA
ttattttaccggtctgatgtgggaatcaaatgggatgatgcgtgcagaggcctttgtaaactctgcctctctctgtaatgcaagctgctatgggtagatggcaggcgagtggggtcaacaaggttttcatctcaacccttgctgcacatgcagctctggggaaaaatgatgccctggaatcatctgcagagttgagggtagggcctgttatggggctttttagtttttaaggctcctaaggtgatgttggtgggcagtaggatttggaaactgctagtaaacaaaaaagttacagatgttacgagaaagcagaacttgtggattagggtgagcaggcaggaaaagccttgaaggaaaagatgggaatctgagaagtagaagggacccaggccaaggggagatggtaaagagaatggagtgaagacaagggaaaatgagaaaggggaatgaaggatagagggggagctggtacatcaattaatcacaaagtgggtggcacctggtctggcgaaggctgcacctcttccacgaagcctggtgactccaaatgtctcttattggaagcttcatgtcaccttgtgccaccctgtctccccattgccatattgttttataattgtttgtgtgtgtatctgtctccctcagtggatcaggggttctttgagggcacaaattatttctttcacctcagcatctagcaaggttcctggcatagaccaagaacttaatacataatgtctaaatgacaggtattgagcatctggtaaggacttatcttaggtctgactctgccactaatttactgggatgtctacaacccactattttgcctctttgggtttcaaggcccccccccacccgccaccaacattgtgaaatgtgagttctctaggaattcaagtatgtaggtagcactgtgtctagaaaaaaatattcagtgaggtatcaggctcaaataattagaacacattaatttaggtcagtgcttctcaagctttaatgtgcctgctaaggtacatggggactttgtgaaaatgcagattctaattcagtaggcctggagtgggtcttgagatgccgcatttcacacaggctcccagatgatgtcaatgtggcctgtcagcagagctcacttgagtagcatgcgtcaatagtacttactttcaggtgagaccaatgctgctggattttgactctcacttggagtagcaggtggttaggctgcgttcgtatgataacaaaatgatctgctgaaaatgtcccactccagaccctgaaataatgatttttttttttttaattcttggcagagatactttgttggctagaattataggacataggtcaaccaaatctgggctcaaatcctgttcccaacgtttcttaattatatgattttaggctagtaacctcaaaatcttagcctcagtgtcctattctatcaaatggtatcacagaacttatctcaagaagtataactcatagagcatgataatttatatgaggatgtaggtaacgcaattagcatacggtgggtgctcagtacatgctaattctcttcttactgacttagcaacacaggaagtaaagagatttcaggaactataggtttgttcttttcacatcgagaaaggaagcttaagtagtataggaaatgcagtacgtttcacttcctttcttcagggaacataaaacccccaggtgtatcttagaacaggatcagtaagagagaaatagtaaaagtgaaaagttctcagagggagctgactcaccactcctcagggagaggtaaccaccctgtgaggctttgtgagaaaggtggtctgggcagcttgggggaaatatgaaggaaaagacaaagatacaggaactctccggctcacaacaaaggccttctttcttcaacactccattttcaacactgaactctgactcttgttggacttatctttagacacattctatttatttgattagggtagaggaatcagaacaacttcatatcctggccttctgagaggtctggtatgagcacaggtattgttatctgacttgatctttactgtggtccctttcagtaacctccataataatttcttaggttcctaaataacaagctctaagggccactcctggggtgattttcagtgacttcccatcagaatagttgtaatcagcctttgtcaatcttccactgtcccttttgcacctgtcttccaatttccaggcacctgttaggcccaagctcacagtaagctgttataatgacatctctttgggacctcaatcaatagctccctggtcaggtgtgtgcatgctttgtatttagctctcctgtactctacatttgctaatcctagccaagcgttaagaaaacaatctgattacagatgtaatcctgtgaccatctgcttgatttatcactcccagaaagaatttgcattttgaaaaaggagcttcttaaagtaaatgagtctttaattttgtgggttattacaataactgctcattttatatatattgagggaatttaaaaacatgcaaatgtgcaggcctcaaaattttgcattttaaaggtctccattaagctttgagcatctgtagtttaaaaaactttccaggtgattctgatgtacacccagagttgcaaactcctggcaattggtcgattatgtatgggacattaaaacacatttttattttttatttttgtctattcttgacctaaacatccatcaattatccattgctgtgtaacaacacaccctaaaacttcatggattaaacaaaaaataacaatacttctcctgaatctgtaggtcgctttggctcagcacgcagctcttctctgactagtagtatcaactgagtcactcctttggctgtatccagctgatgtctgggctgggttggaagatctgagaagacttcgttcacatggacagtgcctcattctcttccatgtggctggtttgtttcttttcacagcatggttgtctcaggttaatcagatttcttagatggtggctggctttatgaatgaaagcagaagctgccagacttcttaaacgataaacctggaactggcatagcatcacttctgctatatgttactcaccaagccaagtcaaaagtccactccgggtttaagagagtaagaagcagactataaattttggtgggcagagtatcatgtgcatagaggaaaataatcaattcatggcatcttgtttggagactatttactgtaacgtcttcccatcaatttgccaaataatacgaacttgcagcataacaccttacctctgacgaatgtcaatcctcatgatataggagttaagaagaaattacttaggcagatagtgaggctacaggagtccttggtaaagttttccttttaatgaaaagcagcccccaaatcattttcttttctaacaaagagcagcctgtataattaagctgcagacatcgataagcaagctggaagcttgcacgggtgaatgccaccagttgtgccgataggaaaaggctacctgggactaggcatgttcaaaatagtggctccatcttcccttctctttgccaaaccatgtgtacagtaaggagcagacaatatggcccctgctaggcaaagacataataataagattagggtggggcaaccagcctttccctctgcactatgtaaacatcacgcctggtcgaaccaatctgtgagccttacgtaaatcagacatacctccttaagcctgcctataaattctggtgcagtccaccaaggggtgttttttttttcccttttgggagctcctctgtctcacaagggagagagctggtctcctcctttctctttcttttgcctattaaacctctgctcctaaactcactcctctgtggtctgtgtccttaatcttcttggcacaagatgatgaaccctgggtatttaccccagacaatgatgctgcttcacttacgccaaacaaaaaaagcaccgaatgtccatatggaccatctaagcattcaagacgatcaatgagtctcttaaaaaagatttgaggactcatgggtttcctccctctctaatattgctgtagatgtggctcaattgatcagttctaacaagaggaacctcaggcaatcaatcactccgcttacatttttttcagtttccaaagcctaagggagttttgtgggaggagagtataaccaaatggaacatgggccttgagcccaacagaaacggtttagactcttagatctgccagctgtgagagacctttggtaagctgattctctgagcctctgttttggcttcagaaaagtgaacgtgacatcttctttcaggctgtttgtgaaaactcagggtgatggtgttcgtagatgtcacctgaactgtttagcaaatgttgcaccttttgctactaacatctctcttttgtaaaagacataactgtagaagtatttaaacttacaggaaaaagaaagcatttcgcaaacacttctgggacaaagaggagagagagagaaagctttgtgtatatttaatgagtaaaagcatataatttaaaatgtgaaagttaatatcttgagtgagtcacccaaaatgaattctctgatattgataagcattgaacgtagtttagttagttttccagaacaatgattctaccctccttggaaaatagcttctaggaagtttagtgaggaagccttgagaaggcgtcagcactctaagccaggtgggctgtagagttacaatctgaagcaaccggtagagagaaaaaaagaagaatggattacaaaagatgggcaaggtgcctggaatttcacacctacatcatctcctttgtggtaaaggggaaatcccaggatccatagctacccctgatatactagtatatccttgacctattggagcaaaactatagagaacaatgactgcattatttcacaagacaccctttgatccagtttagaacactagttttccattcttctgt

SEQ ID NO: 34 - DNA, 5000 bp, Homo sapiens
  source 1..5000: mol_type = other DNA
caaatccagtctgctgccccctgcttttgtaaacaaaatttaacccggaacaagtctgaagtgtgattcttcagttttatgaggtggccctatttctaattttgcttcactcgaagctgtcatccaaacatttcaatttgaccttgttccaccaacaccccattggccctccagtgctcccactctctccacatctccatgtggcagtttcaaatctctgcccacagttccctagcttcctacccctgcaataagagtcggactttgcttcgtctcactccccaaatccctgttgcctctgtgaccttgccaaatctcctctttcagaaatgtacctcatcctgcctcctttcacctggctcaaatgtgtctcctccaggaaacctttttgatgctggtatccctcttctgtgtcccaatcacctgccatgcatttaccattgctacctgtctagtttgcaggcagggcatggtggcagaagctagccttctttcgggacactcactggccatccccactgccaagaacacctgcccttccggcttctcttccctagcccactcttattcctgaaagtctcatttgaaatgttactgcctcagaaaagccattcctgaagcccctgcctatactccaaggtcaaggcctgtgtcttcacagccctttttcaaggaatttattccaataaaattagatggcttatggtgtgattatttattaatgtcttcctacccattaaacagacagcttcatcaaggcaagccagccatctgttctatagaccaagtgtttcctcagacttggtccacaccagtagacagttttctagtgaatgaatagagttaacgtcactctaaattaaattaatcaagttggagctgggtgcggtagctcgcgcctgtaatcccagcactttgggaggccgaggcaggcagatcacctgaggtcaggagttcaagagcagcctggccaacatggcaaaactccatttatattaaacatacaaaaattagccaggcgtggtggtggcaggtgcctgtagtcccagctactcaggaggctgaggcaggagaattgcttgaacccatgaggtggaggttgcagtcagccgagattgcaccaccgcactccaccctgggcaacagagcaagactctgtctcaaaataataataatattaataattaataatagtaataataataaagttgggaatgggtcagttaacaaaagacaccaagatagaaaccatcgtctccaccagttagcgacacagaggatggatgtgtgcattaggcttttctctgagaataggattttgtcctatccaagaaaaatgacaaacacccatgaagagggttttattttttcttggtggaaatgaaagtattagtttttctaatcaagagtaagtcatttgtccccacttcccctgcaaacaacaaaaaagaacccatgatatttgttgagaaccagcttacagcctaatcctggccctggaagcatgccattctctaatagatacaattaagttaaaagactggggttggtcacatggtttcagacaatggccctatatttaggagttttgtcccaaatataatcagattccaaaggcctgtgaggatttatttatttatgaaaataaggacccagtgccttctgaaataagcccttttagtgcagcagccgtgtacactggggcagatttactctgaaaaataacaaggcttctctgacccaggaaagtcacagcctgaaatgcttatttttgaagcagcatttattttctgctggtttagaacatgttccccttaggaataacctcctggctgccccagaaatgctgatatgtggattgtgtaatgtcagctctcctgtgcgccctatctgtgcctccttcctcgcagcagggaggcgttcagcccgcccctgcccgttccctgccttgctcttttctaagaactggaaattattcctgagatgagaggaataaaacatacataggacatatccagtctatttctgaagcagaacatggagaaaacctagcgaagagattacaaaaaaagataaaagaagttttcttcctagtcctcactcccaatcccttcctgcccaagtttatgtctgctaagggctcctctcatcagcccaacttcctgggctgctttcaggatcaggagagagaggagcttcataatatttccatgtttactttttaggcaaggataggtaagaagggccccattcacagaagcactgctcagacaggatagcagatggcggctgggtgggaaggaacgaaaccctagaacatttctgtcctcatgatcctcttatgtgcccttctgatcagaacatgactcctgattgattttgaggaaagttccaatacctctggctgtccgcatccttcccttttctcacagagtgtgctcaagtgtgtctggtcatatggtttctgatttacacccaactcctagttcctctttctcaggtggaggccttcaagacctggggaaatgacacatgcctccttcaatgagcagttgtcatcaaccatcagttagctctgagctgagtagcttaaatgtgttaggctggggaaatgggggcattgagagagcaaggcagggctttgccttcattctaaccatacatatattctccaagcagcacagggcaagtcagaagatgtgggtccaagatctgggcaacttgggcttgtgacttcacttctctatgacttagttttcccatctgtaaaatggactaagcacctctcacttcttattttaccggtctgatgtgggaatcaaatgggatgatgcgtgcagaggcctttgtaaactctgcctctctctgtaatgcaagctgctatgggtagatggcaggcgagtggggtcaacaaggttttcatctcaacccttgctgcacatgcagctctggggaaaaatgatgccctggaatcatctgcagagttgagggtagggcctgttatggggctttttagtttttaaggctcctaaggtgatgttggtgggcagtaggatttggaaactgctagtaaacaaaaaagttacagatgttacgagaaagcagaacttgtggattagggtgagcaggcaggaaaagccttgaaggaaaagatgggaatctgagaagtagaagggacccaggccaaggggagatggtaaagagaatggagtgaagacaagggaaaatgagaaaggggaatgaaggatagagggggagctggtacatcaattaatcacaaagtgggtggcacctggtctggcgaaggctgcacctcttccacgaagcctggtgactccaaatgtctcttattggaagcttcatgtcaccttgtgccaccctgtctccccattgccatattgttttataattgtttgtgtgtgtatctgtctccctcagtggatcaggggttctttgagggcacaaattatttctttcacctcagcatctagcaaggttcctggcatagaccaagaacttaatacataatgtctaaatgacaggtattgagcatctggtaaggacttatcttaggtctgactctgccactaatttactgggatgtctacaacccactattttgcctctttgggtttcaaggcccccccccacccgccaccaacattgtgaaatgtgagttctctaggaattcaagtatgtaggtagcactgtgtctagaaaaaaatattcagtgaggtatcaggctcaaataattagaacacattaatttaggtcagtgcttctcaagctttaatgtgcctgctaaggtacatggggactttgtgaaaatgcagattctaattcagtaggcctggagtgggtcttgagatgccgcatttcacacaggctcccagatgatgtcaatgtggcctgtcagcagagctcacttgagtagcatgcgtcaatagtacttactttcaggtgagaccaatgctgctggattttgactctcacttggagtagcaggtggttaggctgcgttcgtatgataacaaaatgatctgctgaaaatgtcccactccagaccctgaaataatgatttttttttttttaattcttggcagagatactttgttggctagaattataggacataggtcaaccaaatctgggctcaaatcctgttcccaacgtttcttaattatatgattttaggctagtaacctcaaaatcttagcctcagtgtcctattctatcaaatggtatcacagaacttatctcaagaagtataactcatagagcatgataatttatatgaggatgtaggtaacgcaattagcatacggtgggtgctcagtacatgctaattctcttcttactgacttagcaacacaggaagtaaagagatttcaggaactataggtttgttcttttcacatcgagaaaggaagcttaagtagtataggaaatgcagtacgtttcacttcctttcttcagggaacataaaacccccaggtgtatcttagaacaggatcagtaagagagaaatagtaaaagtgaaaagttctcagagggagctgactcaccactcctcagggagaggtaaccaccctgtgaggctttgtgagaaaggtggtctgggcagcttgggggaaatatgaaggaaaagacaaagatacaggaactctccggctcacaacaaaggccttctttcttcaacactccattttcaacactgaactctgactcttgttggacttatctttagacacattctatttatttgattagggtagaggaatcagaacaacttcatatcctggccttctgagaggtctggtatgagcacaggtattgttatctgacttgatctttactgtggtccctttcagtaacctccataataatttcttaggttcctaaataacaagctctaagggccactcctggggtgattttcagtgacttcccatcagaatagttgtaatcagcctttgtcaatcttccactgtcccttttgc

SEQ ID NO: 35 - DNA, 5000 bp, Homo sapiens
  source 1..5000: mol_type = other DNA
ttgccgttacaggatagccaaaaacagattttaaaaacaaagagaatattctgtaattctacgacagtcttcattaagctgtcattcatttagtcatccaaattttgaccacatccacgtatcactttcactgttatttagtatttagtaaatatacttttattaagtcattttacttcttttttgcttaaatacatttatttataaagggaaattaatggaaagaaaaaaaaaacacaggagcaaactatagaaacgatcatggcctaagagagaaagcattgctcatgaatgtgttcaggaagagcagacaggaatggatcttgggcttggggaccagtttttcattgtgttggctcaagacattggcatgaaatgagtggaatgagtaagggagaggaagatccaaggcagcaggttgactcactgaaagaagtgaactgcaatctgagaaggaaggttggagtagaacgggatggcggcttttgattgaatggtagggtggggcactgaagctagaaagaggccgtgactgaaaaactagagccaggggtttcgagaaacatttagtttaaggacatcacataagggtcaaagctgaaaaattgggagagcttgaagagatataaagctgctttggggatgaggggatgcttggaaagttagggatgattaataggatttaggaaatggaaactgccatgtagatggagtcatggtgttactgcagcggagtaagccagcaatttggaagtgacagatctttcaggaaacatggtaatggaatatgtgtcttcctcattttaagttttatagcatttcttcctgggattacactctgagagcaggaatgcccatcagcaagggataagaaaagtcaagatataagtcttgggtgaaatagtcaatatatcccacctaaaattattttgcatattgtataatagtacatgtatatataccatatttggggaaactctgctttaaagcattctacaaatacatggaattacttacaattagctcttggaacaaactttattatccttacaattctcctccattcttccctggtaactatatcctggtgtctaggaactaaaaatgatcccatgggcacagttggctttgtgtttatatggtatctggggagccgctggaggattgggcgtggtaaaattataaacttattgaagttcaaacttagaacaccttttaacatctggcaaatataaaccatctcctattttaatttttgtttttgggcctttctcaaaaaactaaaagttcttgctctaacaatcttggcttagataggagacggtctgaagtaaatgcggtagaaacagttacatcataaactattccaagacctgtcctaaaatgggaaatctaggagatggctcggtgctccatcttgctggattcccacactggaaagccaaggacaaaggaggattaagtattccacgcattatcttgggattggggtttcatgcttgggaggccggcgcctgaagtttgcctaagaacagacagacagcagcagctcaaagcctgatctatacaactttatctagagcagggtcatttgaaaggatagtggtcattgctaattttgagcatttgcataaacaggaggaaacaataaggaaattgtttaagaaggaaaaaaaaaatcctatagccctcccctccatttctccctcccttccttcctttcctgcaaatgactgcttgtttaataccaaactaaccattgtgcttttgagaatccatgattactgcttgggctggaaggtagtaagattttgaggctagaggggaggtcagaagtcatctaatttagtcctcttactttgaaataagaaacttacagcccagtgaagtgaaatgatgatcccaaggtcacccaattggttagtagcagaatcaggcctaggtcagtattccctgcctccatctgttgtaaatcctcttggacaggctttgtggcatgctaccagcaaagcttattggaaagcatttaagaacgttagagcgtctgcagatgtgattttacaggttatttatttgttttactgcagatcatgacttcccctccatccctcttcttgtttcagggccagattctgccctttgatgttctttgtggcattcttgttgtctttggcccgaatctgaagcagggcttgacccaaagctactggtgcctttgtttgctcccagaggacttcccctcctcccaggagactcagaaaaatcagttgtcttagaaatactttctacacaagatgcatcctgctgtgtctgtgtcattgaatctttgagttggaagagacctctgaggtcatcagtccaacccctgacccggttcaggcatgtcttccaaaaattccctggcatatggtcacatagtcttgtggctggaaggtacctcagagaccaaagaatatcttgatttctttttgtaggtaaagaagcttagattcaaaggtgaaatgattcatctccatgcaggtggcagagctaaaattgtaattcatgtttctgaccctgagtctgtagcaccgtccactgttccatccaacttctaattattcttctaacaattgtccagtgaattatccctgtatttatttgttaatcataattaatttgctaagccgcccttattttaccctgtactaatagttaaggaaaataggcatttaactagataaaaagaccacacaagaatggtcttcattccctaaaacaatcgggggtttttttttaaggttcttttgggtttttttgtttgtttgtttgttttgtttgtttgttttttgagatggagtctcgctctgtcgcccaggctggagtgcagtggcacaatctcggctcactgcaacctccacctcccgggttcaagcaattcttctgcctcagcctcccgagtagctgggactacaggcacacaccaccacgcctggctaatttttctttttttagtaagacagggtttcaccatcttggccaggctggtctcaaactcctgacctcatggtccacctgcctcagcctccccaagtgatgggattacaggtgtgagccaccgtgcccggccattttgggtttttttaatgctgctgtaacaagttatgaaaaatttggtttaagccaagacatatttatggtcttagttctggaggtcagatgactgatgttgctgggctaaactcaggctgctggcagagttgttttccttttggaggctctagggagaatctgtttccttgcctttttcagcttccagagactgcaacattcctgctcatggtgtggcacctgcctccatcttcaaagtacattactctaactctgcttccatcatttctgactctctctcgcctacttctttgctttataaggcccttctgattacatttggcccatcttaataatccagaaaaatccccctatttcatgatccctaatataatcatatctgtaaaatccgttttgccatgtaagctgacacattcacaggttcttggaatttggacctggatgactttggaggagcattattctgcctaccacagcgactgttgacaaagcagtccagcgctgtcactgacatattaagatataaagtcgttgcagaaaacagatgcttgaaccaagttgtgagggatgtgaagcgttcactagccagggcagataaaatagagaatgatgagtagaaagaagagcatgttcagagctgcccagatatgagagtgagcacatggcacttttggggaatgctgagtactcaggatgactaggaaagactattatcctattaaaaatgcattcttaaagtctcaacaagtttcacacataatgcagttaggataagagatgcaaagagatatccacctaactcaaggtaatattgtaaatggcacattcataaggattgaaagaggaggagtataatgagagagggaggtttgtatcttaagtctgctgttgagggacaagaataatcagaaagggtcaagtggtgtaaactgcccaaaataggaagtctggagaggcctcgagggtaagaaatatttatttttaaaaaagaagagtgcgacagcacacataatcttttgcggggaggtgggggcttaactatgtgatatcacatatgggaaacattcattgttgtcaatcaaatgctagaagctgctgaatttaggtccaatggtgactctcaaacttcatctctgcacagtagcttatttaaaaatttacttggggttgtctttggctatacttcatcatcatgatctgttccaagtttacagaagagagaataaacaaatttaaaaaatatattttttcaatttggaggttttccctcatactttttttgcaccttcttcaactttctttctagcaagtacatatccaaatgaatgaatggtaataaatataaaaatacagattttatgctaggtcttgagatgtgaagaactgcaatatgtattagagtcctatttctgactatactatgaatagatgggcccatagttcattgggtcccacaaagaacatcatattcccacatgctctattaccacaatttggatggcaattacatttttatcagcaagcagttattgacacgaattatgtctcaggttgtcttataagctcaagggatagaggaacatataaaatattgcattctcaatggatagttttatatcttagacctaggacctagagatgacctaggcaatactaattttttcggcaccctccaattcactcccaagtaatttacttgcatcctttagctccttgaatcctctttacccaaattttgtacaaaaggaacaaggaagagtagcaagctcatggcaggagctcaatcaatgggtttggaacaaataatgaggacattaaagtaaaaatttggaggtaaattgcctgtgctcacagagctattaattggcagtgctgaacttgaacccata

SEQ ID NO: 36 - DNA, 5000 bp, Homo sapiens
  source 1..5000: mol_type = other DNA
ttatgatcatgatctgtatgacctgacatgacttgatctcgactgctgacttcgatgtcctcactctcccttctgtccctgctcattcaccattgtccataaacactgaactttcttttgtcccttgaacatttcaaactcattcctatccctgggctttgctcatgatattgtctacttggaatagctcccctcatttctgcctggctcacttcttctctgaactaaaatagtatcctcttcctggttactctcagaaatatcaccctatattatcctctcaattacatttatcattgcttaaaattatttaatttatttatctctgtatatgttcattatctatctctcctcatagctgagaagatagttgctccacaatatcccacctttagagttgtgcatggcacatgatatgtgcttaataaatattcgtggaataaatagacgaatgaaaaacacaaaaatgaattaaaatcgtggtaagtactgtgaattaagcaaaaggtgtcatggaagctataacagtggggacttgcataagactaggtagtcactgaaggactttctgaagaagtgacattcaagctgagacctgaaaggtgagttagagtaagtgcaatgcgggggaagaactgtccagtccagggaagcagcatgtgtaggcgatctgaggccatgaagaacttggcgcattagagaaatgaaggaggccggtatggattgagtggagagcatggcatgagatgagtttgaaggccagtagaagtcacttatagaaatgtggtaaatgctgatgtacagtcatgcacaaaggaggcaatgcctggggaaggtaagctgctggagctggaggaatggaattcaagaaagtctttacagaagcagcaatattgaggctgagaagtcaccagaacgatgggaatggagaagtgagaggagtacagcagggaggcacacgcaagggcagacaagtctacaacaagctgcatattctgggaaccacaggtatggtgtatcaagaaaggcacagctaagaggaacactcaggtgactgtcttgcttgtctttatatctctagcccactaggcagttaatcaatattggttcattgaataaggaaaaaaggaaatacaggagacaaaactggagaactaggtagaaaccaagccacaagcatccttctgtccttactaagggtatgaaattttattcagtaaaaataaaacataataaaaagtcttatgatagaactgacatgatgagatggaaaacccagtcaatctcgtattttctcagagttttaaagaggggcccacttattgattaatttagtaagaagtttatcattcatctacttgattcaatgctgcacacactcacaatttgctcaatataattttattgtgaatcttgagccttgtggtaattttacagaatacattaggttctttattctaggattgggatgtggagttcaattctaagcaaagttttctattgggacaattgcaaatttaaaacgattagctttaaatagagcattgttccttatcatggattattttggttcttaaatgtctgtcaaagtaataataggagatttgagctattttaaatatttcaaaagtccaacacatttatcaatgattaggttgcacatactaattaaatttcaaggttctttagtattagctaaaattacactaatttcaatgtgtggtcacgctgatgagcatgaatgacagtcttaagtgatgatagcctctttgctgagcagtgtccctgtgtaaagatccacaactctccaatcatctttggaggacctgagatcaatttgcatgaaatagtattcattagacatttctgggaaagaactcatcctaaggaaaagttcagtcaagtagctcaagttcaggcaaatggcaatgaaggactgttgaagggaaggtaggatttataacacaaaactatctgggtggtcttagaccttagtcactgagagccagggctttggaatgttacatatcttcacttgaatcctagcttaccaattctttctgggtaactttggacattggcccaatcctgtagagcctcagttttctcatttgtgaggtgggaatcatcatgggtactatgtaagtttgtcttagggaggggggctatgcatgaaaactattgctctaagttctccataaagaaaaaagtggctatgttctgtcctactctgtctaggccttctcaggccttcaaccattgcccatgcccacagcccagtcatcatcatccacttagttgcaatgtgactccacgcatgtcctggccaattcagtaacctctacacaatgcagcaagatggcactggcagtatggcacctagatcctgggtgttggagccaggcaacatggcttgctaaaggcaatgtacagtaggcaggatttgaagacaactgctaataacctatacccttttattatcccctcctgttaagtgtgaaatgacctgttaatatgatgatatgtggcaaaagggattttatagaggtaattgggggccctaatcagttgagtgcactgaaagggagattatgcaggtgggcctgacccaattacatgagctctttaaatctggatctacagattagagacaaatgaagtcagagactcaaagcataagagatattcaacatgatagagactctccactgctggctaagacagagggagccatgtggcaacaaatagagtgtattttagtggccaagaatggcagcttcagatggcagccagcaaggaaatgggagcctctgtcctacagcaacaaggaactgagttctgccaacaaactaaatgagctggggagaggatccctcgttccagatgagatgagcttgagtttggtaaacattatgatttcgagcttgtgagtccctaaacacagagcctagctaggccatgctcagactgctgactcatgtaaactgtaaactaacaaataggtgtaattttaaactgctaactttgtgtgagtctgtcttagagcaataaaacacaagtacagaataattgcctacatggagtttctgggtagttaggagcatgagagaatctctctggggctttctggcacctggaaggccttccctatggactggcagaatacttcctgttcgagcaagtctctccaaagcaatggggatcatgaaaaactgaactcattgacaatcccccaatctgcttagttgtgttagactttgttaaatatgcatttattatgcatatcaagtatatatcagtcatgcagcagacagatacaaaacttacactgggtaacagaggagagttttataaatggactctatgcaaaggtttggagacatacacatgggagtgttcagtaccccaaaggttagcaatagcagtgaactgtcagtacccctagggaaaaatctcctgttttctagatctctttcccagctgacttactttgatggtatatgattcccaacagctttctgagtaaggctgtctgatagacaacagttttgggaaacttgagtgtctaaaaatgtctttgttatctcctcacacattggactaagggtataggattctaggttggaaaccatttttgctcaggtgtttgaaaatattattccattgtcttctagtttttgatgttgctattaagcccaatgtcattgtaacctttcatgctttaaaggcaattttttattctattgcatcgcttagtatctttttattcttggagttttaaattttgtcatgatgccccatgatgtggatctttcttctttcatgacgtaggaaacttgttaaatccctttagtctagaaacccatatatgttggttctgtgcatattttttaaaaattcctagataatttcctcccatctaatctgtcatttttttttcctggaactaatatttgttagatattagacccccagaggattgtctaattttcttacctttctccagtattgtctatctcatcaacttttattttttcaaatttctgaagagttcttcaacgcagtattccaattatcctccctttttcttgacactttaatatttgctttcacaacaattccaaggatatttttattttgtaaatgcttaccttgttctctgttttatatattcaagattttcctcaattattttacgatttttggcactccactaataactagaatggagcactggaaagttaattagaggtgctgtgtatgtgactgaggctgtcaactggttgacctcaccatagagtgatgagaagaggatctgccattttattgtgatagtatctgcatgtttgttttcctcctgagacggatagtctccatggagaagaattctccagtgtcacagttggagaatagagacctgaatgacagtgtcagggaaaaatcataaatagaacttttcaaaatgtatattttgacttaattttacattacaaaaatgtaattaaaattacatttttgatatatcttcttgtatccaccctcagatagtcctagtgtctccatgttctctggtcttctgtaatagagcctctgtagatagtaagtgttggggaaggagttatttggctattaggactagacagtgaatctgtggttctaactcttccttacacatattgtcaatcaaccctctcatttttctcccctttcaaatacactggatgccaatgagccattatcttttctgggtcttctgatgttaactagattttgacatccctaatatggacttagttttcagcttgctctgctctcatcagttggctacctctatccttttgtttcccatagttaacaatttagttgacatctcacaactgatgtcattacctttccaatcctctttgttcttgtaagtttatatcatttcttcattcctttacaatcattttattggtttgcataaaagaacggagtaaaagcatagattcaagccgcca

SEQ ID NO: 37 - DNA, 5000 bp, Homo sapiens
  source 1..5000: mol_type = other DNA
ggaaggtgagattattactgactacatgtggagaaactcatttgtaagtggctcagtggctgttcctagttacgcagtaattcagtggcagggctggtctccaaacttcctgctgcaagaaagatccacaactgacccagctgattgtaaatcattgagagcaaggacaggatctgattcatctctggactcagggagatattcatcattcacaactggcaatcatgctctccagccaaccaagatggaagcctcctagaacattctggtgcgcaccagctgagaggcagctctgcctgtcttcctgagtgcctgaaacacagctgacactcggcaaatgcttgttgaatggaattaaatttcatatcaagagagtatgactgattaagaaagaatacctcaagaaaattgctactgacaaaaacgtttttgtagttccctattttccctttatattaatataaatttaatattatttattgagcacctactatatgccaagctatatgctagaagctcaatatattccatgtaattaagggttgaaaatgaaaattttacaaattcattatctttgggctccttttcgcagtggatggatccagagaggacttgtgaatgaatgaagctttgatggctggaggtcatggatttctcatttaagataagaagtggcatcctatgccagatggtttccttcctctgcccttgcacgtgttttttttttttttttcctctaccaggaagggcaccacgattctctcttgtgtttggcacattccagtttagtgctcacagctctattacggcatccagtaccctgtaaagtctgctttcaccctctatcagctactctcaatcatctctcctttttgtgatatttttaaactttgtgcatgtccatcaagacccataagaaaatgtggcatattatcagtataaagagctatctttcccaggagactaagaattcctcaagggaagaagacttcctcctccttgtcctgctcctcctgctcctcttcctcctgctcctcctcctccttcttctttcttcttctttccttctcaagaaggaatgataattccagtgcctttcgctgacacattctagatgctttctgtcaactgagtgatgctcacttcagctcctggacctgaggtgtcatctgccttcctgcccatggagatctttcactggatatctaactgctgcttgtgtggcctctcaggcagacatcatgcctcgttcattttttgcatacctatgtagtacttatacatctatcgcatctttcatctcaaatcctcatggcattccccctctgtttgttatcattacacccagaatggctctttgaggtctcacagtcattttcttcaaggtggagaaactgtggaccaggagaaattctgggtcttctcaatcagtcatagaaggctgaaaatgcaattagccattctcagggcaaatagaatctctgaaaggcatcaaaggaagctccttcctgcgtcttcctttccttgtccttcttcttttactctatcttctctccctccccactcatagatatataaaactggaaaggattaagaaatcctgtcacttaaaactaatatttgacagatgaggttagagaagatgggcagattgggattctaagtccttgattgtcttcttaaaaatgataccactgagaccccttatgacttttacttgtgtggatagggttaggccctgccaccacttccatttatcttatttcctgaactggctgcttggccttgtcttggagaggcaacacgtccactggtaactccttccatctctctgcacatgccaccactcactgcttgtttatcaagaggtagaagaattttactcaaaattgtggcttaaaaatgaagcaaaataaaaccttgattacagagacatattcagacatttggatttgtcactaggagtagcggatcagcattatgtgctaattccatgacatcatgcatacgttacattgataaagagacactgatattgggtgtctacagtatgccagctaatttatgtataattaagttaaaaggcaaatataattcccagtgtatagatgaggcaattccagctgaataagtttgagggttctaatgtaagggcgaggcgtttgggctctggagtcataaagacacattagtacttccccatctgagctctgctacctcctcaccgtgcaactgtacgcaactcatcctttctgtgcctcactttccttgtctgaaaatgtagataaacaacgctttccagatcaagttgttttgaggattaaatcgaaaaattaatgtaagaaggtttagcactgtgcctagcacatgccaaatccttcttaaaagtgataattgccccagatcatggagctgctaagtgtgatgcagaggactttgagacaggcagagttttgtttctacccttgcttctgccatcttgcctgcaatgttgggttggcatgagaattatagataatttgaataaaaaacctagcacaatgactgccctggaataaatggaaactccattcttgcagttggctcagggttaaaaaaaaaaaaaaaagaaaaaagaaaaagaaaaattggagtcatttttgacctttctctctcatacctccctgataccatcagcagatcctctggctctgccttcaaaataagtctatgattggatcatttctcaccgcctctgctgctgtcactaccttgtttggattactacaatggccttctacctgattccccttgctccaccatccccaactttcagcctagtttccacttagcagctacagtcaggttggagaatgtgagttagatcagtaactctgcatcctcccacagcagctttgcaatttgctcagagtagaagctgaaaacccaataaaggcttacaaatcctctgtgagctgcaaaccacacccaaccccagcttcagctgtcactgtttgccacttttatttattggcttttgttttgttcatcaaacaccagaataccttaaacatttgcatttgctgttgcaatgctcacgcctcagatattgacatgactgattcctcttgtctcttgaatttttctcaagcgttgccctattgatgatgcctttcctgaactccctatttaaagttatacctggtttccatcgccatcgccaccgcaaccatgctgagctctctttcccactttctttgctctatgttgcgcctaccgtgagctaccccttgagtgtatgaataatgatttgatggtttctactacttattgtcaaaatattctagtatctaattctgagttgtttggctgtgagactgaatattctacttaatttttccttggcagtgaaatatgaacaatgatacatataaggttctgcatgcttgtacttggtcacttgttttttgagaagtaaataaaacaccatttccaaacagtctaaacagagtggctgacatacaccaggagtcagttaatagtaattgtttttcatttgattaaatgaattccatggccaggcacggtagctcacacctgtaatcctagcactttgggaggctgagggaggcagatcacaaggtcaagagttggagaccagcctggccaacatagtgaaacccctgtctctactaaaaatgcaaaaaaaaaaaaacattagccgggcatggcagtgtgtgcctgtaatcccagctactcagcaggctgaggcaggagaatcgcatgaaccaggaggcgggggttttagtgagcccagctgagattgtgccattgcactccaggccaggcaacagtgtaagactctgtctcaaaaaaaaaaaaaaaaaaaaagaaagaaagaaaagaaaaagaagtgaatgtgaattccattcaatttggcttgacaatgatttattgagttatattttaaacttcttaatagagtttcccttttaaaaggcataccaattataaatacagaattagttgtgaaaatgaatatttactcagaataagatatcacaagttgcacaatgaactaaaattaacaaatataattaatgtcagaaaaatctacatagtaatattttaattaatttcttttaaacacaattgaataaaacttctttgctacttttttgggctgcctgtcttttcaaagatctccctgccagatgacagtggtgtctcaatatcattttctttacagggaatcagaagttaactcagtctttcctctagcatggttgactggaatttgtgtttattattgatagttgggatgcataaagcatattacctcacccacaggcagtcatgctatttgtagtactgctagagatgtgtgccctataaatattggactgctgatacgttctattttgtgattgcactcgaagaaaataatgcatgatgcatttataattgtatctgctgcacagtctgcacatcctgtgagaaaagtcgctccgcgtcgactgggcatcctgactttcttattgctttcactgttgatcctgtcccactttaccaaatcaagccttgtctcttacacacagactcatactacaaagggttgtgctgagatgtgaaaacttcagagggactatttgaaatattgtttaaaaaagcttcatctaatcaagatgccataatgtcctctctctccacccccatatatgacaaagaggtgtctttctaatgaaatttattttttaagttaaaatatttagaaaatggataatctctttagctcaattgtctgttcacattaattgcattaacatttatatatgtatgaagaaaaaatttaaactcattaaactttatgatcacagaaaattgaatgtacacacatatctatgtgcataattatatgtacagtatgtgtataattttgttgtagagatgtgtgttggagtaatcaaagaaaaaccctaaagtgtggaaaatatgtgaatataatattctctggagaaaga

SEQ ID NO: 38 - DNA, 5000 bp, Homo sapiens
  source 1..5000: mol_type = other DNA
taagctcaaatcttagttttgcctcttacaaactatgtgactttgggatgattcatttatttcatccttctggacctcagtttctttatttgtaaaatgggaaagcagccacggagaacacaaaagcaagtgagtttggccatgtttccataagactttactgatggagattgaaatttgattctcatatactttttacgtatcacaaatagtattcttttaatttttgcaaccattaaaaaaatgtaaaagtcattcttagcctctgggcaggctgcactaaaacaaagggaggccagatgtggcctgaggactgcagtttgctgaaactgctctagtcatttctctcttcctggtcaggggctgcagaagctgagtccgttcttccagaaccttttgtcttcagaacttatcactcaagtctgggaggaaggcaagaggccctcccttttctttgtgctgttgaggcagctccggcctccctctgagaactgcacagggagagaggccttttctggctgcttttctcccagtctcagctcacacccaggtttttggcattgtgttagataattttgctagtagatgactggtccgtacaactctcattatgggggcctctgtgcatgaagatgtgaatgtctaaagtgaggggaacttcaggccaattattaagcctgggaaagttcacgcatctgaaaaattatgcataggctatgtatatgcacatcacctactccactcctattatacacaccccaagaacatggtaaaaacatgcaagaaaaaaaaaaaaagagccagactggccaggtgtggtgactcatgcctgtagtcccagcactttgggaagccaaagtgggtggatcgcttgagctcaagagatggagaccagcctgggcaacagggcgaaaccctgtctttacaaaaaatacaaaaaagttagccaggtgtggtggcgcacccctgtagtcccagctacttgaagggttgaggtgggagggtcgcttgagccgaggagatgggggccgcagtgagccgtgattaactgcaccactggaccccagcctgggtgacagagggagaccatgtctcaaaaaaagaaaaaataaaccaacaaaaaacccaaccacaacaaaaaaccagccagacctttcttttcattatgaaaatgctacttcttaaaacgtctgcatcattgacagtatctcctttatgtcctggaatgcgagaaggtagcacactgtatatgccttttcagggtgggagggggagaaagaatggagacacaggcccagacgagagacctttatcatttcctctaggaatttttcttggctctctagggctggggattgacagggataatgaattcatggctcagctggcaaatccaattaagcttattaacagggtggccttgccttgggcctgctctgtggtgggaccttggcagagaaggaagtgccttctgtgcctggggcatctgatcccctcttaggcagccgtggaagctgcagttagggcatttcaagggacagcaggactgcacacttatatagagtctgtggtgcataatctagaactaggatgaccaaccatcccagttttcccaggaccaagaggtttttctggaaaaaaataacaaagtctaggaaaaactgggacaagttggtaactttaaatcttgcctcaggaaaaaatgaggcccattgtgtccttgaagggatgtaagtaatctcctatgtagatcctctagaacaggcatcagcaaactttttgtaaaggacaagatagtcaatacttcaggctttttcgggccacatggttgctgagtcacaactactcagcttagctgtggtgatgtgaaagtaaccatagacagtatgtaaatgaatgagtgtggctgcggcttcatgaagtgttatttataaaaacaggccagatttggctgtgggctgtacaatgccaatgcttgctcaaggaggtatccagtttttctacctctctgtatgccacacatacatacacaaacaggcacacccgctgtaacattgtttgggaagcaagacagcccagtggttatagagggcttgaactttggagttagaaagatctcaacttgaatcttggctttcccacctgctagttgggtgaccttgaggatcgcatctctgagcctccgagcttcagtttcctcatctagaaactaggaataatacaaatgggatcgccgggcacggtggctcacgcctgtaatcctagcactttgggaggctgaggtgggtggattgcctgagcgcaggagttcaagaccagcctgggcaacacggtgaaaccccgtctctattaaaaatacaaaaagttagccgggagtggttgcatgcacctgtaatcccagctacttgggagactgagacaggagaatcgcttgaacccaggaggtggaggttgcagtgagccgagatagagccattgcactccagcctgggtgacagagtgagactcttctcaaaaaaaaaaataaataaataaataaatacaaatgggatctgcctgccaagatagaaggaaaggggatttggacttcatctcttgactggggaggtgtcagagaacatgcagttgtctctaattcaccacaagagcgggccgggaggggtgacaaggttattcagcaatcgatgcctctgtatctagctcccagctagttgctgaagccaaaacactcagtaagatgacaaataggtgttttccttgtggaattcatggtcacatagaaaatttagatagctaaacagccagttatagtaaacagggttgaagtccagatagtgagaaggcaagagtgaaggaggcttctgattctttaagagaaaaaagattcgatgagggaaatacatcccttttgctgccttagagagcaaagaagagcagctgctcaggagccgctagggttggtcacatgtgcaggtggcttctctttgtgtgaaactgggtgaggctgtttgctgcaggggtgtgggctcaggatgtgtatggatatgtttgtgtgcctcccggtagataacttgtgggctgatgacagagaaagggcagctctgatgtaacaagagacatgctcacatctgtgacttgaagaagccgagactttagggccagaagggtcttagagatgcctcatccaattccttgtcctcttctcattttacaggtgactaaatggaggacttgtccaaagcatctcagctaattggccctctgattcctcatcaaaataacatatttcacaagcgaggccaattagattctgggtgaactcagtgattgagttcagatactgttcactagctggcctcacctgatttggggttgacagaagaaggcagggtgaatgttgagaagacaagagtgtatgctcatcagagaggactgggcacagtgccaggcacagttctcagaaatggctggttgaatttgaacctgcatttattatgcttcatctaaattgcattcaaagctaacattagaaaatttcaacctaaattaaaattcttacattttcatttaaatgaataaaacccccaacttaaaactgcgtaaactaagagggtgatctcaggggagatgctgttgtgtttcaaagataacctcaacgtgtttaaagtatgagctgaaaatcttggatgagtttcagaagatagaaagaacctaaaagcctaaaagcccagaatcaagctatgttttgttgagagaagcacactggcattcttgacctggactcttggtcatggtagtatgaagctcaaccacaaggcatgttgctcaatgatgtcctggcctcctcatgtggccatctctgatatagctatgttataggataccttagggaatgtcagttcctctgcctggctgaagaggcatgatgtctgagtcagtcccatagtgtgagaatctcttctccaagataagatgacgtcaagtatggaacccttggtacatgcctggcagatgtttggtttgtatttccatccgggagatgccacccctgggcttgtcctgaaaacaactaagtccaaggggttattagactgcttacagaaggcttcatctagaaagtaaaaacaaaacttttaatgtatgagccatgcataatcattgctaaaacataaaaacaaaacagagaaaatctaaaaggaaataaaaattacactattcaaatatgactacttctaaaattttcatgtgtattctcccagaattttttctatatgtgatatgaatgtattttttaggcaaaattgagagcacactgtaaagctctatgacactgaacaagtgacatgaatttcctgaggtttaggactcttttcaacacaataggagtgatgataccataaggattattgcttcctctcaaaatgctgctgccgtgccgtaggaggcccaaggcacatgcacaggccatgcatgagtgctcaaagaatcgaccctgctgagcccagccttaaaatcatcccagcccgggcatcggccacatgagtgaaagaccctgggtgattccagccccctgacatttgagtcacccataactatttgagtctttctaaattagaccccagacactgtggagcagagaaaagccatccccactgtgctctgtccaatttctgacccacagaagcagcgtgcataagatggtgattgttttactttacattttgcatatggtgtggtttgttttgcagcaacagtgtctataactggactgttaactttgaggagcactttagagcaaacccttacctgtgtgtggcatccctttccatttcctcaaggaacttgttcttcgggttgttttctctctatcctgactcttctgtccctcctactccttccattcagcctaaaagtggagtcaagccttctgcaaggggaaaatcccttcccctgtctccatcattttgaactacaatctttccctcaccaattcctcatca

SEQ ID NO: 39 - DNA, 9002 bp, Homo sapiens
  source 1..9002: mol_type = other DNA
gcagaaggagcgcgcgcgcctgggagagctcggggtccggcgcttgcggtaggagccacgagccggagagaggggtcccgggctgcctcgaccgccgtcgccaccgcctctcctgtcgcgaccgcagctcccaccgcgccggcggccgtcgtcgccgaagccaccacagccgctgtgtgcagcctggaagggggggcgggggggagggggggagccgcgaaagcggggtgccgaggactttgcaacttgcccaggaaggtggaggggtgggagggggagggggacctccgcacgagacccagcggcccgggttggagcgtccagccctgcagcggatcatggtgcagcaggcggagagcttggaagcggagagcaacctgccccgggaggcgctggacacggaggagggcgaattcatggcttgcagcccggtggccctggacgagagcgacccagactggtgcaagacggcgtcgggccacatcaagcggccgatgaacgcgttcatggtatggtccaagatcgaacgcaggaagatcatggagcagtctccggacatgcacaacgccgagatctccaagaggctgggcaagcgctggaaaatgctgaaggacagcgagaagatcccgttcatccgggaggcggagcggctgcggctcaagcacatggccgactaccccgactacaagtaccggccccggaaaaagcccaaaatggacccctcggccaagcccagcgccagccagagcccagagaagagcgcggccggcggcggcggcgggagcgcgggcggaggcgcgggcggtgccaagacctccaagggctccagcaagaaatgcggcaagctcaaggcccccgcggccgcgggcgccaaggcgggcgcgggcaaggcggcccagtccggggactacgggggcgcgggcgacgactacgtgctgggcagcctgcgcgtgagcggctcgggcggcggcggcgcgggcaagacggtcaagtgcgtgtttctggatgaggacgacgacgacgacgacgacgacgacgagctgcagctgcagatcaaacaggagccggacgaggaggacgaggaaccaccgcaccagcagctcctgcagccgccggggcagcagccgtcgcagctgctgagacgctacaacgtcgccaaagtgcccgccagccctacgctgagcagctcggcggagtcccccgagggagcgagcctctacgacgaggtgcgggccggcgcgacctcgggcgccgggggcggcagccgcctctactacagcttcaagaacatcaccaagcagcacccgccgccgctcgcgcagcccgcgctgtcgcccgcgtcctcgcgctcggtgtccacctcctcgtccagcagcagcggcagcagcagcggcagcagcggcgaggacgccgacgacctgatgttcgacctgagcttgaatttctctcaaagcgcgcacagcgccagcgagcagcagctggggggcggcgcggcggccgggaacctgtccctgtcgctggtggataaggatttggattcgttcagcgagggcagcctgggctcccacttcgagttccccgactactgcacgccggagctgagcgagatgatcgcgggggactggctggaggcgaacttctccgacctggtgttcacatattgaaaggcgcccgctgctcgctctttctctcggagggtgcagagctgggttccttgggaggaagttgtagtggtgatgatgatgatgataatgatgatgatgatggtggtgttgatggtggcggtggtagggtggaggggagagaagaagatgctgatgatattgataagatgtcgtgacgcaaagaaattggaaaacatgatgaaaattttggtggagttaaagtgaaatgagtagtttttaaacatttttcctgtcctttttttgtcccccctcccttcctttatcgtgtctcaaggtagttgcatacctagtctggagttgtgattattttcccaaaaaatgtgtttttgtaattactatttctttttcctgaaattcgtgattgcaacaaaggcagagggggcggggcgggggaggggaggtaggacccgctccggaaggcgctgtttgaagcttgtcggtctttgaagtctggaagacgtctgcagaggacccttttggcagcacaactgttactctagggagttggtggagatatttttttttcttaagagaacttaaagaactggtgatttttttttaacaaaaaaagggaccattgcaacttttgttaatttaattttttttttttttttttttttttttttttttggagggagaaaactgatgtcttctatgcatccgattcttaacaaaactgcagggagcttgaaaaaatgcagactgtacaaacgcttacaaaaaaaaaaactgtgaactgacttaagatcagagtttacttttcagatcaaattgtttatggttttacaaatgtgatttctacttgccaactttttttttgtaacttgttcccttatacctccttgattgaataccagacagcctagacctcagtacaaaaggtattgaaacatttttgatacataacagacctcagtcttttttaaaaattaatatattttcaggcgtatttttgtacagtgaaaagggaacattcttgctgtgttttttcagtaagactttcaggcacttcttcccttttgatttcttttttttcctctgttttttagcatgcaagtatgttggtacgttatgtcctggtttaaaaaggattaaaattttaaaataatccttgcatctaaaggccttgtggtttaaaaaaaaaaagcaaacttttttttgtacagctatagtagagatttgttcaatatttgtaggtaaagatttattgaaaatggtgatatagacctcagagctgttatcttagtttaaagattgtatatgtactgtactatagtaggactttatgtatctcatacgctgtgatgtggatggggccccagatggaaggtttgaaactggattctcgatttttagcaaaaaagaaaaaaaaaaggcacatagtttaaaaagtttctcattttgtgcaatataatctaaataaagtacagaccatctgcatattttgtagcaaatggtggcaaagcagactcaatgcactgtcgacatcattgcctgtttttttttttttttttttgtgctggaagtctgtatcttgacaattttaataaatcagctggaactgatagaaactcgcatcgccaatagtctctatggaagtcaaactggaggtcctgttgtcgcagagcattcggtggtgaggctgttgtgtgcgcggatgaggggaggtggcaggagagaattctacatttagggggttaggctgaaaagtgttcaattagcaggctgatttctttttcctcttccgctagttgtgaaagacaggggaagggtgttctttctctctgccctccctttccatctccagctccccatttcctttctcacctcctcctccactccctgcctcttctccccacccatcctggcgggcgggctgcgcggaggctcgggagctggccggggaggggcggatggaggggcctgggttgccagctcccttggtcggggtcctgctcgctggggcttgtgtgttctctgcggcgggccgcgtccccgctgagcctcgcggtgacagccgcctttggcagcgagcgctcggggcacttctatccccgcctctcaaagggtggggacagccgtttccagatttgaattttttctgttctttatttttaacgctgcatcttcgcgtgtgctcagaggtggttgttggcggagaacgccgccgcagtgtttgacctctagcggtgaagggggaaggggaagaggaaaggagagaagtggtcggtgtctgtttccttctgtcccccggggccgtggagctgtcggagggaaggaggacggtgcggggccgcagggggcgcggggcgcggcgggacccaggctacgagcgggagggaggcgggagtcgggggaagacgcggcaggccggccgagggcaccccgaggaacatggcatggcctctgtgcgatccgagtcgcggtctccggggtgcctgggagggccgaaccactggtgagggcgtggggagcagggggtggcagagggcacccgggcggtagtccgggacgcgcaaggcagagccctgacgctccgggtccccgtgcctggctcttcttgcctcgccaccgcgtgctcctgggcgcgccccgccgcgggcccttgaggcgcgcggagacaccagcgctggcttcccgggcccgcgggccggggagggaagcctcggggctgcggggtgagaggaagaaagcaaacccggggagcaggcggctgccgcacccgcgcaccccgggccctcaccacgccctccccgcgcgccggctcaggggctgccccggaatcagctccccggggccgccgcaacgaaggtggatccgcatcttgattgttctccgggagcctcctgggggctccggcggcggcgcgggcgcgacccatcccgctggcgctccccgctcgctgaaccccgtttgcctgtccacaccccctcgctccccaccatttttcctgaccggcctgtgtccccgagccctcgcggcaggcccgagcaggcgatcgcggccgggcacgcgcgccccgggctcccgccccccttccgagcatccgccgcctcttttctgctgggtctgggaggagggaggctgggaggccgctcggggcccagcgtgccagccccggagttcagcctcccgagctgcggcgcccgcagcggaggaggttttcagtggctgattgaaactcactgcaaaatcaccacgactctttcacctactgagatgattgaccgaggtttggccttccatttttactgagatttggcgagaccgaatggaagcgtccgcacagtaactgcagctgctaggccagaggggccccggcgcccttcccgcctcccctcccgcttgcttttgccttactcgatcttaccaccacccctcccccggccccccgactgagaactcgggcctctcacccgccccccagcctcccgctctgggcgagcctcctccccagcccccacccctgggatgcgaagccagcaagcttttgctgcagatggacaggtttcttttctgtggctttttcctttcgataaaccatcagatttcagtagtacatttgggaaaagaaggggctgatggcgttaaccaggttctcaatatagaactggatttctggagttgtttaccttaccccacaccccctcaacatgtagactaatgcagccattggtggtacatttattttagccacggataattgaaccagcggtttacaattgacacgtgctccgtgctggtgattttatgtggcagccctctgctgcagttccgaaacttgttggcaacgtaaacccattgataggctgatctatgtattttgaaagcctgaaaacttggcatgtcttttctgttttaatcatagatgaatcttggacattttctgtggtgaggtggaaactttaagtaaattagtaaagtaataatttggcttcagaatgggaagagatagtcaagattttttttttttaaagccatgtggcctaacttgatacaaaaataaaagtaattgtttggcaatctaaatttaaaacctgttagaactcaggacaggcgcttcaatgcgctttttaacaatatttaaggctgttttgatgagtgcgttgtgagaatcatcttaatgaattctttattgagtgtctaaaacatagtataatacacatggtattcttgccactggatagtcttcaataaaagtttaattgatttttttttgttggtctcttaagtaagtcttatttttaactaagcattgacagaatatcttaaaatggtaacctgggggtggcgggtgggtgctgtgtgcacggcagcctagccagtggggatcctgctgtttattataagtagttcacagactctgatggcattttggtaagctttccatctttaagaaattgaaccagcattctcttattaattctttaaactgtggaagtaatttccagttcttacactctgatacgcatcccttttatttaaaaaaaaaaaaaatgctaataaaaggcagtgtacttaaactgtgctttgcaaatattgtgtatgttatgaatgactacagacactgggcaaattatttgtagaatgattatcctttagctagagaaagaaatcattacaactcttttgggcagagatgtttctttttaatgttaatcaaggggaagtgatttaaatatgcataaatgtagcagtcagggtgatttagttgcttttttcatgaaagaaaaagactcaaaagacaagacttatttttctcttctgggacttgaaatcataatcatctgatattagtacagtacaagaaatttacatttgttttttacttcagaatttaagtgacttttgcccaaggaatttgagaaataaggcaaataagttgctctattttaaagtagtcattcaatataaatatattatatcaatcttaacttttttattctctgatatgattaataatatgtatattcttacttttcttctaatgggcatatgtatccttgtggacactttgagagaggttttcttggactctcccatttatagaatctttatactcttttactgtgtggttccctgcttttaacagatttctgaggcaaatatatttgtgcttttttcttatgtaggaagaccagcgaaaatagtttactgagttgtcaattttatcagtagataagaaactttctttattacagtttcagggaagattttttcaggatatttctcagttattctaagggccaaattttgtaaaatttccattaggaatgtcagtttcaaataccctttgtatagcctaagcctgtgaggataacaagaatgagccttacctatcctaacacagggatttacaagttcccaaagtaaccgtctccatgtaactcttgacatacttttctgagatttggcttatttttattattggttatttctcactgttcattctatttgatttattctacaacatccccttttatttaatgatctggaaaattctgctctttgataacaactcaggatttttttgttcagttttggtttttgccccttcctgtggagcctacattttcaaccacaataaagatgaaacaaaatttatgaaactgagctctcttccattttacttactgctggcttttttttttttttttttccttgattcctaccataccttcgtttttttcattgtacttttttaacactacctatatccattagctgcctaattagttttatctgttccatgtggatgcagtgagtttataagagaatttcacaaacaagtagttttttagtgaacttaaaataaacagaattttaaaggagacctatttttatactcaataaaagcacaaaagtgcagaaagtataaaacggcttacaaagggagacacaagctcataatgttccatgtataaaagtaataactttattgggtagagatattcttacaagatctagcacctctgccagtgcacagataggactgttttaaatgatttgggaacttttggttgcctgcagttgtgaacagagaacttctctacagagaaacaaaccactaaaagcaatatgaccgagttgagatgtggtttccaatgagcaattggtgaatttaagcaacctggatgtgcatatgtggaggctcccgtctcactgtttgatcaaacttcttttatgtagtcacgtagacttgattttttctgctgtgaaaatgaaaaaataaagcaatatgacaaaaagtttaaaaatgcataaaaaataggatttcctctaggctcctcgaagagatttttttaatatgatgcttgtcttactttcttagacacgttacatttccccttccaaaaaaaaaaaaaaggacaactggaagtaatttatcatataaagaattttgatcaaatagatattgacaaagggccctctgtcacatttttcttcatccagcttttgttcaaaaacagtatgcctcctcccttgaatcacatagggagaaacgttatactccattctcattaatttcccattttgtctacttttactcttgtacatatgttgtgggtttaagagtcttttgcatttgttctgtgacacctttttttgaattgactgttttaaaacggaggcctattttttccggtttgggactcctagtggttatggcatcccataatgcttcgtgacggccaccaggacagaaccacctgatgttttagagcagttttcagcatgacactgttaacaagtgtgtattttccaaggccacatgaaacttactttcttagccactccaggtttgggagcagaaaagctgaaaaacccttttgtgtagaagtctgagtggtttgtgggggggaccttttttagagtttgcatgccagcgcacggcctattgctgtgaaacagagagaaggtaaagctacctgaggcagtgcgctggaggatgaagtgtttgatagcactaggggggaaagaaaatgcatggcaaagtttcgtcttctcgtagactatctagcatgcagagtgtagtgtgttgaaacggtgtatgacattgctgtatcaaagttgtaaaattaagcattatttattgaaaactatgtatttttttgtaaaaacctgatcacatagagaatatcagtggcttgtgcttgtgcttcgatctaaccagcttcttgacccacccccccttggtatgcagtgttaatgctcagggttgaaaatagtacactccaatgtctcttttgcaagagtttttcacagaggattacatttgttcaaaagactctaataaaattgtgtgatcaatcttca

SEQ ID NO: 40 - DNA, 2240 bp, Homo sapiens
  source 1..2240: mol_type = other DNA
catttcgtcttagccacgcagaagtcgcgtgtctaggtgagtcgcggtgggtcctcgcttgcagttcagcgaccacgtttgtttcgacgccggaccgcgtaagagacgatgatgttgggcacggaaggtggagagggattcgtggtgaaggtccggggcttgccctggtcttgctcggccgatgaagtgcagaggtttttttctgactgcaaaattcaaaatggggctcaaggtattcgtttcatctacaccagagaaggcagaccaagtggcgaggcttttgttgaacttgaatcagaagatgaagtcaaattggccctgaaaaaagacagagaaactatgggacacagatatgttgaagtattcaagtcaaacaacgttgaaatggattgggtgttgaagcatactggtccaaatagtcctgacacggccaatgatggctttgtacggcttagaggacttccctttggatgtagcaaggaagaaattgttcagttcttctcagggttggaaatcgtgccaaatgggataacattgccggtggacttccaggggaggagtacgggggaggccttcgtgcagtttgcttcacaggaaatagctgaaaaggctctaaagaaacacaaggaaagaatagggcacaggtatattgaaatctttaagagcagtagagctgaagttagaactcattatgatccaccacgaaagcttatggccatgcagcggccaggtccttatgacagacctggggctggtagagggtataacagcattggcagaggagctggctttgagaggatgaggcgtggtgcttatggtggaggctatggaggctatgatgattacaatggctataatgatggctatggatttgggtcagatagatttggaagagacctcaattactgtttttcaggaatgtctgatcacagatacggggatggtggctctactttccagagcacaacaggacactgtgtacacatgcggggattaccttacagagctactgagaatgacatttataattttttttcaccgctcaaccctgtgagagtacacattgaaattggtcctgatggcagagtaactggtgaagcagatgtcgagttcgcaactcatgaagatgctgtggcagctatgtcaaaagacaaagcaaatatgcaacacagatatgtagaactcttcttgaattctacagcaggagcaagcggtggtgcttacgaacacagatatgtagaactcttcttgaattctacagcaggagcaagcggtggtgcttatggtagccaaatgatgggaggcatgggcttgtcaaaccagtccagctacgggggcccagccagccagcagctgagtgggggttacggaggcggctacggtggccagagcagcatgagtggatacgaccaagttttacaggaaaactccagtgattttcaatcaaacattgcataggtaaccaaggagcagtgaacagcagctactacagtagtggaagccgtgcatctatgggcgtgaacggaatgggagggttgtctagcatgtccagtatgagtggtggatggggaatgtaattgatcgatcctgatcactgactcttggtcaaccttttttttttttttttttttttctttaagaaaacttcagtttaacagtttctgcaatacaagcttgtgatttatgcttactctaagtggaaatcaggattgttatgaagacttaaggcccagtatttttgaatacaatactcatctaggatgtaacagtgaagctgagtaaactataactgttaaacttaagttccagcttttctcaagttagttataggatgtacttaagcagtaagcgtatttaggtaaaagcagttgaattatgttaaatgttgccctttgccacgttaaattgaacactgttttggatgcatgttgaaagacatgcttttatttttttgtaaaacaatataggagctgtgtctactattaaaagtgaaacattttggcatgtttgttaattctagtttcatttaataacctgtaaggcacgtaagtttaagctttttttttttttaagttaatgggaaaaatttgagacgcaataccaatacttaggattttggtcttggtgtttgtatgaaattctgaggccttgatttaaatctttcattgtattgtgatttccttttaggtatattgcgctaagtgaaacttgtcaaataaatcctccttttaaaaactgca